(19) United States
(12) Patent Application Publication
(10) Pub. No.: US 2024/0417730 A1
(43) Pub. Date: Dec. 19, 2024

(54) METHODS OF MODULATING RNA TRANSLATION

(71) Applicant: The Regents of the University of California, La Jolla, CA (US)

(72) Inventors: Eugene Yeo, La Jolla, CA (US); En-Ching Luo, La Jolla, CA (US)

(21) Appl. No.: 18/782,680

(22) Filed: Jul. 24, 2024

Related U.S. Application Data
(62) Division of application No. 17/512,270, filed on Oct. 27, 2021.
(60) Provisional application No. 63/106,631, filed on Oct. 28, 2020.

(51) Int. Cl. C12N 15/113; C12N 7/00

(52) U.S. Cl. CPC C12N 15/113; C12N 7/00; C12N 2310/113; C12N 2310/141; C12N 2310/321; C12N 2310/531

(57) ABSTRACT

Provided are methods of modulating gene expression of a target RNA in a cell comprising (a) recruiting a modulation unit, wherein the modulation unit comprises an RNA binding protein (RBP), an exogenous RNA binding moiety, and a gene-editing agent; (b) delivering the modulation unit into the cell; and (c) detecting change in the target RNA translation, wherein the modulation unit modulates gene expression of the target RNA in the cell.

### CROSS-REFERENCE TO RELATED APPLICATIONS

This application is a divisional of application Ser. No. 17/512,270, filed on Oct. 27, 2021, which claims priority to U.S. Provisional Application Ser. No. 63/106,631, filed on Oct. 28, 2020. The disclosure of the prior application is considered part of the disclosure of this application, and is incorporated herein by reference in its entirety.

### SEQUENCE LISTING

This application contains a Sequence Listing that has been submitted electronically as an XML file named “15670-0349002_SL_ST26. XML.” The XML file, created on Jul. 24, 2024, is 6,319 bytes in size. The material in the XML file is hereby incorporated by reference in its entirety.

### BACKGROUND

The fate of the transcriptome determines the status and health of a cell, and RNA-binding proteins (RBPs) control the post-transcriptional processing of these mRNA transcripts. Dysfunction of RBPs is linked to dozens of multisystemic diseases, cancer, and neurological disorders. However, despite their association with disease and although the importance of regulating gene expression at the cytoplasmic stages of an mRNA life cycle is well appreciated, only a small fraction of the over 1,500 RBPs identified thus far have known RNA targets and molecular roles. Rapid, large-scale assignment of molecular functions to more than a thousand uncharacterized and emerging RNA binding proteins (RBPs) is a critical bottleneck to a complete understanding of gene expression regulation.

### SUMMARY

The present disclosure is based, at least in part, on modulating RNA translation in a cell.

Provided herein are methods of modulating gene expression of a target RNA in a cell comprising (a) assembling a modulation unit, wherein the modulation unit comprises an RNA binding protein (RBP), an exogenous RNA binding moiety, and a gene-editing agent; (b) delivering the modulation unit into the cell; and (c) detecting change in the target RNA translation, wherein the modulation unit modulates gene expression of the target RNA in the cell.

In some embodiments, the exogenous RNA binding moiety comprises a MS2 bacteriophage coat protein (MCP). In some embodiments, the gene-editing agent comprises CRISPR components. In some embodiments, the gene-editing agent comprises shRNAs, siRNAs, ASOs, or microRNa mimics.

In some embodiments, the delivering step (b) comprises lipofection. In some embodiments, the delivering step (b) comprises a virus-based delivery. In some embodiments, the virus-based delivery comprises adeno-associated virus or lentivirus.

In some embodiments, the detecting step (c) comprises using a reporter mRNA. In some embodiments, the reporter mRNA comprises a luciferase mRNA. In some embodiments, the target RNA is an endogenous mRNA. In some embodiments, the target RNA is a non-coding RNA.

In some embodiments, the RBP is BTG1, CNOT2, CNOT4, CNOT7, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP1IL, YTHDF2, ZC3H18, ZCCHC11, ZFP36, ZFP36L1, ZFP36L2, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPE1, HNRPDL, HSPB1, KIAA1324, LARP1, LARP4, LARP4B, LIN28A, LUC7L, MAK16, MATR3, MBNL2, MEPCE, MRPL39, MTDH, NDUFV3, NUFIP2, NUSAP1, PABPC1, PABPC5, PCBP4, PEG10, PPAN, PPIL4, PRPF3, PRPF31, PRRC2B, PTRH1, PUS7, RBM33, RBM38, RBMX2, RPLIOA, RPL14, RPL15, RPLPO, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, or ZRANB2.

In some embodiments, the gene expression of the target RNA is upregulated. In some embodiments, the gene expression of the target RNA is downregulated.

Also provided herein are methods of identifying a function of an RNA binding protein (RBP) comprising (a) contacting the RBP to an exogenous RNA binding moiety; (b) allowing the exogenous RNA binding moiety to interact with an RNA structural motif; and (c) profiling the RBP tethered to the RNA structural motif, thereby identifying a function of the RBP.

In some embodiments, the exogenous RNA binding moiety comprises a MS2 bacteriophage coat protein (MCP). In some embodiments, the RNA structural motif comprises a reporter mRNA. In some embodiments, the reporter mRNA comprises a MS2 genomic RNA stem-loop.

In some embodiments, the profiling comprises transcriptome analysis or gene expression analysis. In some embodiments, the profiling comprises enhanced cross-linking immunoprecipitation (eCLIP).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows a collection of 1,062 open reading frames (ORFs) for 888 unique RBPs and their overlap with those identified experimentally by Baltz et al. (Baltz et al., 2012) and Castello et al. (Castello et al., 2012).

FIG. 1B shows an exemplary schematic of luciferase reporters. The coding region for either firefly (top) or Renilla (bottom) luciferase contain 6 MS2 stem-loop structures in the 3′UTR. The complementary reporters lacking MS2 hairpins were used as internal controls to normalize reporter signals. RBPs fused C-terminally to the MS2 coat protein (MCP), which recognizes MS2 hairpins with high affinity, are co-expressed with the reporters in a HeLa cell line.

FIG. 1C shows time-course analysis of the activity of the luciferase reporters in the presence of co-expressed known negative regulators of RNA stability (CNOT7 and ZFP36) or negative controls (EGFP and the FLAG peptide), with (‘-MCP’) or without MCP fusion. Values are expressed as ratio of the median luciferase activity of MS2-tagged over untagged reporters in the presence of the indicated RBPs, relative to that of the ratio of MS2-tagged over untagged FLAG controls at timepoint 0. Left and right panels in FIG. 1C correspond to top and bottom reporter pairs in FIG. 1B, respectively.

FIG. 1D shows an exemplary experimental and analysis workflow. The screen was conducted on 888 MCP-tagged RBPs in two reporter contexts. Levels of MS2-tagged luciferase reporters were normalized to untagged co-transfected controls reporters. The effect of RBP recruitment was calculated as the ratio of normalized luciferase levels in the presence of MCP-tagged RBPs relative to that of MCP-FLAG control.

FIG. 1E shows hit discovery, wherein RBPs with effects at estimated FDR <0.01 in both reporter assays were considered candidate regulators.

FIG. 1F shows qPCR validation of reporter levels for 35 candidate RBP regulators. Means (n=3 independent measurements) of log2-transformed fold-changes of reporter mRNA levels, calculated analogously to FIG. 1D, were plotted against the corresponding log₂-transformed fold-changes of reporter luciferase levels. The line represents the least-squares linear regression fit. Shaded areas denote the 95% confidence interval. R², Pearson correlation coefficient.

FIG. 1G shows examples of our 50 candidate RBP regulators that are known to affect RNA stability and translation.

FIGS. 1H-1I shows validation of the FIG. 1H 9 negative and FIG. 1I 6 positive candidate regulators of RNA stability and/or translation by repeat luciferase and RT-PCR measurements. Values were calculated as in FIG. 1F. Error bars denote mean±SD for n=4 replicate transfections. *p<0.05 (two-tailed Student's 1-test) vs. FLAG control.

FIGS. 1J-1K shows volcano plots showing the distribution of fold changes for 50 RBP hits from the FIG. 1J Renilla and FIG. 1K firefly reporter assays.

FIG. 1L shows classification of candidate RBP regulators by manual curation.

FIG. 2A shows domain structures of 14 candidate RBPs with RNA destabilizing (left) and stabilizing (right) effects in the tethering assay, with lengths of their polypeptide chains.

FIGS. 2B-2D show histograms showing region-based fold-enrichment of read densities, normalized to paired SMInput controls for (FIG. 2B) BOLL and IFIT2, which show read density enrichment in 3′UTRs; (FIG. 2C) DDX6, MEX3C, TOB2, and TOB1, which show read density enrichment in 5′UTRs; and (FIG. 2D) UBAP2L, which shows read density enrichment in CDS.

FIG. 2E shows bar graphs showing eCLIP binding cluster distribution across transcript regions for the 8 destabilizers and 6 stabilizers. Peak assignment was performed using stringent enrichment criteria (≅4-fold-enrichment and p≤10⁻³ versus SMInput). The average region distribution of the entire transcriptome annotated in GENCODE v19 is indicated at the top.

FIGS. 2F-2H show example genome browser track views of eCLIP read densities (in reads per million, RPM) and corresponding SMInput read densities for (FIG. 2F) BOLL and IFIT2, which show peak enrichment in 3′UTRs, (FIG. 2G) DDX6 and MEX3C, which show peak enrichment in 5′UTRs, and (FIG. 2H) UBAP2L, which shows peak enrichment across exons.

FIGS. 3A-3B show volcano plots showing the distribution of fold changes in transcript levels upon modulation of (FIG. 3A) destabilizers and (FIG. 3B) stabilizers, with distribution histograms shown at the top. FIG. 3A shows depletion of DDX6 (left) and overexpression of TOB2 (right). FIG. 3B shows depletion UBAP2L (left), and overexpression of BOLL (right).

FIGS. 3C-3D show bar plots showing the percentage of overlap between genes significantly up- or downregulated [log₂ (fold change)≥1.23 and FDR-corrected p≤0.05] and significantly bound (≥4-fold-enriched and p≤10⁻³ versus SMInput in eCLIP) upon knockdown (KD) or overexpression (OE) of candidate (FIG. 3C) destabilizers and (FIG. 3D) stabilizers.

FIGS. 3E-3H show cumulative distribution plots of transcript log₂-transformed fold changes of overexpression versus vector control or shRNA-mediated knockdown vs non-targeting control, as indicated, for the destabilizers (FIG. 3E) DDX6 and (FIG. 3F) TOB2, and the stabilizers (FIG. 3G) UBAP2L and (FIG. 3H) BOLL. p-values were calculated using a two-tailed Mann-Whitney U test.

FIGS. 3I-3J show genome browser views from shRNA-mediated knockdowns showing RNA-seq reads and cCLIP reads for (FIG. 3I) MEX3C at the NSMF locus and (FIG. 3J) CLK3 at the NELFCD locus.

FIGS. 4A-4B show translation monitoring using puromycin incorporation. FIG. 4A shows Western blots of extracts from control (WT) HEK293T cells and two independent clonal isolates with CRISPR-mediated disruption of UBAP2L. FIG. 4B (Left) representative anti-puromycin western blot of extracts from puromycin-treated WT and KO cells. GAPDH served as loading control. (Right) Densitometric quantitation of blots from of n=3 independent experiments.

FIG. 4C shows a polysome profile of UBAP2L. (Top) Absorbance (at 260 nm) plot of a HEK293T cell lysate fractionated through a 10-50% a sucrose gradient. (Bottom) Western blots of UBAP2L from corresponding sucrose fractions.

FIGS. 4D-4E show global transcript association with polysomes in UBAP2L knockout cells. FIG. 4D shows scatter plots of log₂-transformed RPKM ratios of polysome transcript levels (y-axis) and input transcript levels (x-axis) between the UBAP2L knockout HEK293T lines and WT samples. The RPKM values from the two replicates were averaged prior to analysis and transcripts with average RPKM≥1 were considered. Numbers and percentages of transcripts in each quadrant are indicated. FIG. 4E shows cumulative distribution plots of log₂-transformed transcript levels (RPKM≥1) in pooled polysome fractions from the two UBAP2L knockout HEK293T lines and WT control, normalized to levels in the respective input lysates. p-values were calculated using a two-sample Kolmogorov-Smirnov test.

FIG. 4F shows a bar graph showing log₂-transformed ratios of input-normalized polysome transcript levels (RPKM) between the two UBAP2L knockout lines (KO) and control (WT). Only transcripts with RPKM≥1 in all three samples were considered (n=9,692). RPKM levels for the two KO lines were averaged.

FIG. 4G shows gene ontology (GO) analysis for UBAP2L exon target transcripts (n=1,425). Significantly enriched GO terms were determined by Fisher's exact test at a false discovery rate of p<0.01. Shown are GO terms that are related to mRNA translation.

FIG. 4H shows a heat map showing log₂-transformed polysome association ratio between UBAP2L knockout lines (KO) and control (WT) for the indicated translation regulators.

FIG. 4I (Left) Representative western blots of UBAP2L, EIF4G1, EIF3B, DDX54, and EEF2 in UBAP2L knockout cells. GAPDH served as a loading control. (Right) Densitometric quantitation of blots from of n=3 independent experiments.

FIGS. 4J-4K show quantitative fluorescence-activated cell sorting (FACS)-based reporter assay for mRNA translation using RCas9-fused UBAP2L. FIG. 4J shows transgene expression constructs. RCas9 is expressed from a tetracycline responsive element (TRE) reporter. A constitutive promoter drives a polycistronic transcript containing puromycin N-acetyl transferase (Puro) and the reverse tetracycline (tet)-controlled transactivator (rtTA) separated by a P2A self-cleaving peptide, as well as CFP fused to a nuclear localization signal (NLS) preceded by an internal ribosome entry site (IRES). A second construct drives rCas9 fused to UBAP2L in same plasmid backbone. rCas9 and rCas9-UBAP2L constructs were integrated into the genome at random copy number to establish stable cell lines. A third reporter construct harbors a U6 promoter driven single guide (sg) RNA targeting the indicated sites in the YFP reporter, which contains of a YFP fused to histone H2B driven by a tet-inducible promoter, and NLS-fused RFP driven by the EFla promoter. The reporter construct was transiently transfected into rCas9 and rCas9-UBAP2L-expressing lines, and the expression levels of the three reporters were measured by FACS. FIG. 4K shows a bar graph showing mean YFP levels in rCas9-UBAP2L expressing cells, normalized to rCas9 expressing cells, on each targeting site.

FIG. 4L shows a bar graph showing ratios of YFP/RFP mRNA levels in rCas9-UBAP2L expressing cells, normalized to rCas9 expressing cells, in the presence of the gRNA targeting site 2. Transcript levels were measured by qRT-PCR and calculated with the ΔΔCT method.

FIG. 5A shows domain structures of UBAP2L constructs inducibly expressed in UBAP2L knockout HEK293T cells. The ubiquitin-associated domain (UBA) and arginine-glycine-rich region (RGG) are indicated.

FIG. 5B shows autoradiograph of UBAP2L-RNA complexes immunoprecipitated from UV cross-linked HEK293T cells treated with increasing concentrations of RNase I, radiolabeled and separated on SDS polyacrylamide gel. Arrow indicates the expected molecular weight of UBAP2L.

FIG. 5C shows autoradiograph of UBAP2L-RNA complexes immunoprecipitated from lysates of UV-crosslinked UBAP2L knockout cells (KO-UBAP2L) expressing the indicated constructs, treated with RNase I, radiolabeled and separated on SDS polyacrylamide gel. Arrow indicates the expected molecular weight of UBAP2L.

FIG. 5D shows a pie chart showing fractions of UBAP2L eCLIP reads from HEK293T cells unambiguously mapping to mRNAs, ribosomal RNAs, and other repeat families.

FIG. 5E shows locations of UBAP2L binding sites on rRNAs. Line plots showing the Kullback-Leibler divergence (relative entropy) for UBAP2L in HEK293T cells and the mean of 446 other RBPs from the ENCODE consortium on 18S and 28S rRNA. Lines show the mean of relative entropy, with light areas indicating 10%-90% confidence intervals.

FIGS. 5F-5H show models of the interactions of UBAP2L on the human ribosome structure. (FIG. 5F) Surface view with 60S ribosomal subunits (RNA and protein). (FIG. 5G) View as in (FIG. 5F) with non-highlighted proteins removed. (FIG. 5H) View as in (FIG. 5G) rotated 90° around the z-axis.

FIG. 6A shows an exemplary schematic of the role of UBAP2L in regulation of global protein synthesis. UBAP2L regulates translation of key genes involved in control of protein synthesis and degradation, including the indicated components the polyadenylation machinery, translation initiation and elongation factors, tRNA synthesis enzymes and members of the ubiquitin pathway.

FIG. 6B shows an exemplary schematic where UBAP2L enhances global protein synthesis by increasing translation efficiency of its target transcripts, as demonstrated (i) by tethered function reporter assay, (ii) by rCas9-fused UBAP2L reporter assay, and (iii) endogenously in cells.

FIG. 7A shows sources of RBP open reading frames (ORFs). The collection of 1062 ORFs for 888 RBPs were acquired from the Dana-Farber Cancer Institute (73.5%), DNASU Plasmid repository (17.5%), in-house cloning efforts (7.7%) and Promega (1.3%).

FIG. 7B shows distribution of known classical and non-classical RNA-binding domains in the RBP library.

FIG. 7C shows a summary of molecular categories for RNA-related functions of the RBP library.

FIG. 7D shows a scatter plot of luciferase effect and RBP size.

FIG. 7E shows luciferase activities from two different reporter constructs. Bar graphs showing log₂-fold changes of the activity of Renilla (top) or firefly (bottom) luciferase reporters in presence of the MS2-fusion ORFs over FLAG control. Each vertical line represents a tethered ORF.

FIG. 7F shows a scatter plot of luciferase activities from the two reporter constructs.

FIGS. 8A-8B show in-line western blots of eCLIP immunoprecipitations of candidate RBPs. (FIG. 8A) Extracts from HEK293T cells or (FIG. 8B) from HEK293T transfected with the indicated MCP-tagged RBP ORFs immunoprecipitated with non-immune (IgG) control antibodies, and western blot analysis using either RBP-specific (FIG. 8A) or anti-V5 (FIG. 8B) antibodies. The molecular weight (in kDa) of standards are indicated on the right. Arrowheads indicate the calculated molecular weight for each RBP or RBP fusion protein.

FIG. 8C shows histograms showing region-based fold-enrichment of read densities, normalized to paired SMInput controls for UBAP2L, which shows read density enrichment in CDS.

FIG. 8D shows metagene maps showing the distribution of eCLIP peak densities at target transcripts. Lines indicate the average number of significantly enriched peaks (≅4-fold-enriched and p≤10⁻³ versus SMInput) across transcripts.

FIG. 8E shows a Venn diagram showing overlap in target transcripts between DDX6 and MEX3C in HEK293T cells.

FIG. 8F shows a metagene map showing the distribution of DDX6 and MEX3C eCLIP peak densities at target transcripts. Lines indicate the average number of significantly enriched peaks (≥4-fold-enriched and p≤10⁻³ versus SMInput) across transcripts.

FIGS. 9A-9B show shRNA-mediated depletion of RBPs in HEK293T cells using 3-5 distinct shRNAs for each RBP, as indicated, compared to non-targeting shRNA control. FIG. 9A shows Western blots, with GAPDH or tubulin serving as loading controls, as indicated. FIG. 9B shows bar graphs indicating RBP transcript levels determined by qRT-PCR, normalized to levels of 18S rRNA.

FIG. 9C shows overexpression of RBPs in HEK293T cells. Bar plots showing transcript levels (RPKM) for each RBP following transfection of RBP expression constructs or FLAG vector control.

FIGS. 9D-9G show numbers of up-or downregulated or unchanged genes for transcripts bound or not bound by the indicated RBP, for (FIG. 9D) knockdown and (FIG. 9E) overexpression of destabilizing RBPs and (FIG. 9F) knockdown and (FIG. 9G) overexpression of stabilizing RBPs.

FIGS. 9H-9I show volcano plots showing the distribution of fold-changes in transcript levels, with distribution histograms at the top, upon (FIG. 9H) depletion of the destabilizer MEX3C (left), overexpression of the destabilizer SNRPA (right); and (FIG. 9I) depletion of the stabilizer CLK3 (left), and overexpression of the destabilizer IFIT2 (right).

FIG. 9J shows a heatmap showing significance in differential expression of genes significantly differentially expressed and significantly bound vs all unbound genes, upon knockdown (KD) or overexpression (OE) of candidate RBPs in each region.

FIG. 10A shows translation monitoring using puromycin incorporation. Representative anti-puromycin western blot of extracts from puromycin-treated UBAP2L knockout (KO1) and parental (WT) HEK293T cell lines. GAPDH served as loading control.

FIGS. 10B-10C show polysome profile of UBAP2L after (FIG. 10B) treatment with 0.5 mM puromycin in vivo, and (FIG. 10C) 30 mM EDTA in vitro. (Top) Absorbance (at 260 nm) plot of a HEK293T cell lysate fractionated through a 10-50% a sucrose gradient. (Bottom) Western blots of UBAP2L from the corresponding fractions.

FIG. 10D shows polysome profiles of HEK293T cells (WT, n=2) and UBAP2L knockout HEK293T cells (KO, n=4) fractionated through 10-50% a sucrose gradients.

FIG. 10E shows bar graphs showing percentages of transcripts with RPKM≥1 of all transcripts with ≥10 reads per transcript, for two UBAP2L knockout lines and control samples (WT).

FIG. 10F shows scatter plots showing correlation of log₂-transformed ratios of input-normalized polysome transcript levels (RPKM) between the two UBAP2L knockout HEK293T lines.

FIG. 10G shows a bar graph showing the percentage of regulated transcripts in UBAP2L targets, and nontargets.

FIG. 10H shows quantitative qRT-PCR validation of reduced polysome association for the indicated transcripts. Transcript levels in inputs and polysome fractions were measured for KO and WT samples.

FIG. 10I shows Western blots of EIF4G1, EIF3B, DDX54, and EEF2 in UBAP2L knockout cells (KO1, KO2). GAPDH served as a loading control.

FIGS. 10J-10K show quantitative fluorescence-activated cell sorting (FACS)-based reporter assay for mRNA translation using RCas9-fused 4EBP1. (FIG. 10J) Plasmid design for RCas9-4EBP1 experiment. (FIG. 10K) Bar graph showing mean YFP levels in rCas9-4EBP1 expressing cells, normalized to rCas9 expressing cells, on each targeting site.

FIG. 11A shows immunofluorescence images showing UBAP2L in HEK293T cells. DAPI is a nuclei marker.

FIG. 11B shows a pie chart showing fractions of UBAP2L replicate 1 eCLIP reads unambiguously mapping to repeat families in HEK293T cells.

FIG. 11C shows locations of UBAP2L binding sites on rRNAs. The line plot shows the fold enrichment of reads for IP over SMInput. Diagram for the expansion segment ES15L shows the nucleotide corresponding to the highest peak in 28S rRNA region.

FIGS. 11D-11G show location of UBAP2L binding sites on rRNA. (FIG. 11D) ES15L (FIG. 11E) ES7S, (FIG. 11F) ES27L, and (FIG. 11G) ES31L.

### DETAILED DESCRIPTION

Detailed herein are methods of modulating gene expression of a target RNA in a cell and methods of identifying a function of an RNA binding protein (RBP). In some embodiments, a method of modulating gene expression of a target RNA in a cell can include (a) assembling a modulation unit, wherein the modulation unit comprises an RNA binding protein (RBP), an exogenous RNA binding moicty, and a gene-editing agent; (b) delivering the modulation unit into the cell; and (c) detecting change in the target RNA translation, wherein the modulation unit modulates gene expression of the target RNA in the cell.

In some embodiments, a method of identifying a function of an RNA binding protein (RBP) can include (a) contacting the RBP to an exogenous RNA binding moicty; (b) allowing the exogenous RNA binding moiety to interact with an RNA structural motif; and (c) profiling the RBP tethered to the RNA structural motif, thereby identifying a function of the RBP.

Various non-limiting aspects of these methods are described herein, and can be used in any combination without limitation. Additional aspects of various components of methods for modulating gene expression of a target RNA, or identifying a function of an RNA binding protein are known in the art.

It must be noted that, as used in the specification and the appended claims, the singular forms “a,” “an” and “the” include plural referents unless the context clearly dictates otherwise.

As used herein, “biological sample” can refer to a sample generally including cells and/or other biological material. A biological sample can be obtained from a mammalian organism. For example, a biological sample can be obtained from a human. A biological sample can be obtained from a non-human mammal (e.g., a dog, a cat, a monkey, a mouse, or a rat). A biological sample can be obtained from non-mammalian organisms (e.g., a plants, an insect, an arachnid, a nematode), a fungi, an amphibian, or a fish (e.g., zebrafish). A biological sample can be obtained from a prokaryote such as a bacterium, e.g., Escherichia coli, Staphylococci or Mycoplasma pneumoniae; an archaca; a virus such as Hepatitis C virus or human immunodeficiency virus; or a viroid. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

In some embodiments, the biological sample can be a tissue sample. In some embodiments, the tissue sample can include live cells from a cell culture. In some embodiments, the tissue sample can be a fresh, frozen tissue sample. In some embodiments, the fresh, frozen tissue sample is cryoground into powder. In some embodiments, the biological sample can be live cells on standard tissue culture dishes. In some embodiments, the biological sample can be flash, frozen tissues that have been cryoground into powder and placed on tissue culture dishes, pre-chilled on dry ice.

As used herein, a “cell” can refer to either a prokaryotic or cukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, “detecting” can refer to a method used to discover, determine, or confirm the existence or presence of a compound and/or substance (e.g., DNA, RNA, a protein). In some embodiments, a detecting method can be used to detect a protein. In some embodiments, a detecting method can be used to detect an RNA binding protein bound to an RNA fragment. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “modulating” can refer to modifying, regulating, or altering the endogenous gene expression in a cell. In some embodiments, modulating gene expression can include systematically influencing RNA stability and/or translation by activating or suppressing the gene expression. In some embodiments, modulation of gene expression can include stabilizing a target RNA. In some embodiments, stabilizing a target RNA can increase translation of the target RNA. In some embodiments, modulation of gene expression can include destabilizing a target RNA. In some embodiments, destabilizing a target RNA can suppress translation of the target RNA. In some embodiments, modulation of gene expression can include increasing translation of a target RNA. In some embodiments, modulation of gene expression can include suppressing translation of a target RNA. In some embodiments, the gene expression of the target RNA is upregulated. In some embodiments, the gene expression of the target RNA is downregulated.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the nucleic acid is a messenger RNA (mRNA). As used herein, “messenger RNA” (mRNA) can refer to any polynucleotide which encodes a polypeptide of interest and which is capable of being translated to produce the encoded polypeptide of interest in vitro, in vivo, in situ, or ex vivo.

### Methods of Modulating Gene Expression of a Target RNA

Provided herein are methods of modulating gene expression of a target RNA in a cell including (a) assembling a modulation unit, wherein the modulation unit comprises an RNA binding protein (RBP), an exogenous RNA binding moiety, and a gene-editing agent; (b) delivering the modulation unit into the cell; and (c) detecting change in the target RNA translation, wherein the modulation unit modulates gene expression of the target RNA in the cell. In some embodiments, a target RNA is an endogenous mRNA. In some embodiments, a target RNA is a non-coding RNA.

In some embodiments, a modulation unit can include an RNA binding protein (RBP), an exogenous RNA binding moiety, and a gene-editing agent. In some embodiments, the exogenous RNA binding moiety comprises a MS2 bacteriophage coat protein (MCP). In some embodiments, the gene-editing agent comprises CRISPR components. In some embodiments, the gene-editing agent comprises shRNAs, siRNAs, ASOs, or microRNa mimics.

### RNA Binding Protein

RNA binding proteins (RBPs) are proteins that bind to the double or single stranded RNA in cells and have important roles in cellular processes (e.g., cellular transport, or localization). RBPs also play a role in post-transcriptional control of RNAs, such as RNA splicing, polyadenylation, mRNA stabilization, mRNA localization, and translation. In some embodiments, an RBP is a cytoplasmic protein. The term “RNA binding protein” can refer to a protein that interacts with RNA molecules (e.g., mRNA) from synthesis to decay to affect their metabolism, localization, stability, and translation. In some embodiments, an RBP is a nuclear protein. In some embodiments, RBPs can include, but are not limited to, splicing factors, RNA stability factors, histone stem-loop binding proteins, or ribosomes. For example, a eukaryotic ribosome can include a collection of RBPs that can interact directly with mRNA coding sequences. In some embodiments, an RBP is a cytoplasmic protein.

In some embodiments, an RNA binding protein comprises a ribosomal protein, wherein the ribosomal protein binds to a ribosome and an mRNA during translation. In some embodiments, an RNA binding protein comprises a ribosomal protein, wherein the ribosomal protein binds to a ribosome or an mRNA during translation. In some embodiments, the RNA binding protein comprises at least one of: SLTM, ZGPAT, PPARGCIB, PELP1, DCP2, CSTF3, TRA2B, ZNF638, SRSF9, LUC7L2, PTBP3, SF3B3, VCP, HNRNPA2B1, PTBP1, PCBP2, LSM14A, LSM12, DHX15, DDX27, DDX17, DDX21, IPO5, RPL22L1, RPL35, RPSA, MRPS34, NIFK, THUMPD1, RPUSD3, RRBP1, EEFSEC, UBAP2L, PUS7L, EIF4ENIF1, BICC1, EIF4E2, DARS2, TRDMT1, UPF3B, ZFP36L2, YTHDF2, EDC3, HNRNPR, UPF3A, ELAVL1, RBM27, XRN1, FUS, EXOSC7, PSPC1, CNOT7, CNOT6, CNOT4, CNOT3, AGO2, ENDOU, RBFOX1 (A2BP1), RBFOX2 (RBM9), RBFOX3 (NeuN), SLBP, RBM5, RBM6, PRBP1, ACO1, Adat1, PCBP1, PCBP3, PCBP4, RBM3, RBM4, APOBEC1, BTG1, CNOT2, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, ZC3H18, ZCCHC11, ZFP36, ZFP36L1, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPE1, HNRPDL, HSPB1, KIAA1324, LARP1, LARP4, LARP4B, LIN28A, LUC7L, MAK16, MATR3, MBNL2, MEPCE, MRPL39, MTDH, NDUFV3, NUFIP2, NUSAP1, PABPC1, PABPC5, PCBP4, PEG10, PPAN, PPIL4, PRPF3, PRPF31, PRRC2B, PTRH1, PUS7, RBM33, RBM38, RBMX2, RPLIOA, RPL14, RPL15, RPLPO, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, or ZRANB2.

RNA-binding proteins (RBPs) have roles in controlling the fate of RNAs including the modulation of pre-mRNA splicing, RNA modification, translation, stability and localization. RBPs are a group of proteins that interact with RNA using an array of strategies from well-defined RNA-binding domains to disordered regions that recognize RNA sequence and/or secondary structures.

As used herein, “RNA-RBP complex” can refer to a ribonucleoprotein complex comprising an RNA-binding protein (RBP) bound to a double or single stranded RNA in a cell. In some embodiments, the RNA-RBP complex can include an RNA fragment bound by an RNA binding protein. In some embodiments, the RBP is crosslinked to an RNA in a biological sample. In some embodiments, the crosslinking can include UV crosslinking. In some embodiments, the RBP is covalently linked to the RNA in a biological sample. In some embodiments, crosslinking can be performed by any method including, but not limited to, thermal crosslinking, chemical crosslinking, physical crosslinking, ionic crosslinking, photo-crosslinking, free-radical initiation crosslinking, an addition reaction, condensation reaction, water-soluble crosslinking reactions, irradiative crosslinking (e.g., x-ray, electron beam), or combinations thereof.

As used herein, “ribosomal protein” can refer to a protein that is present in a ribosome (e.g., a mammalian ribosome) or a protein that binds to a ribosome and an mRNA during translation (e.g., a translation initiation factor, a translation elongation factor, and a translation termination factor). The cukaryotic ribosome is composed of 79 ribosomal proteins, large ribosomal proteins (RPLs) and small subunit proteins (RPSs) that interweave with 4 highly structured RNAs (5S, 5.8S, 18S, and 28S rRNAs) to form the final translation-capable ribonucleoprotein. Thus, quantification of ribosome-associated RNA is highly similar to profiling of RNAs associated with other RNA binding proteins.

In some embodiments, the ribosomal protein binds to a ribosome or an mRNA during translation. The term “translation initiation factor” can refer to a protein that binds to a ribosome, a subunit of a ribosome, and/or an mRNA during the start of translation of an mRNA. The term “translation elongation factor” can refer to a protein that binds to a ribosome, a subunit of a ribosome, and/or mRNA during translation of an mRNA. The term “translation termination factor” can refer to a protein that binds to a ribosome, a subunit or a ribosome, and/or mRNA during cessation of translation and/or release of an mRNA from a ribosome or a subunit of a ribosome. In a ribosome, ribosomal proteins can participate in the translation process and binding of translation factors (e.g., translation initiation factor, translation elongation factor, translation termination factor). In some embodiments, the ribosomal protein is selected from the group consisting of: RPS2, RPS3, RPS3A, RPS4X, RPS4Y1, RPS4Y2, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS28, RPS29, RPS30, RSSA, RACK1, RPL3, RPL4, RPL5, RPL6, RPL7A, RPL7, RPL8, RPL9, RPLIOA, RPLIO, RPL11, RPL12, RPL13A, RPL13, RPL14, RPL15, RPL17, RPL18A, RPL18, RPL19, RPL21, RPL22, RPL23A, RPL23, RPL24, RPL26, RPL27A, RPL27, RPL28, RPL29, RPL30, RPL31, RPL32, RPL34, RPL35A, RPL35, RPL36, RPL37A, RPL37, RPL38, RPL39, RPL40, RPL41, RPLA0, RPLA1, and RPLA2. In some embodiments, the ribosomal protein is a translation initiation factor. In some embodiments, the ribosomal protein is a translation elongation factor. In some embodiments, wherein the ribosomal protein is a translation termination factor.

### Exogenous RNA Binding Moiety and Gene-Editing Agent

As used herein, the term “exogenous RNA binding moiety” refers to a molecule or moiety capable of binding to an RNA (e.g., target RNA). In some embodiments, an exogenous RNA binding moiety can be fused to a protein (e.g., RNA binding protein). In some embodiments, an exogenous RNA binding moiety can include a reporter mRNA. In some embodiments, the exogenous RNA binding moiety can be attached to a protein through an artificial RNA-protein interaction. In some embodiments, an exogenous RNA binding moicty can include a MS2 bacteriophage coat protein (MCP). In some embodiments, an exogenous RNA binding moiety can be fused to an RNA binding protein (RBP).

As used herein, the term “gene-editing agent” can refer to an agent that allows for changing the DNA or RNA (e.g., mRNA) in the genome. In some embodiments, gene-editing can include insertion, deletion, modification, or replacement of the DNA or RNA. In some embodiments, a gene-editing agent can include a nuclease-based gene editing platform. In some embodiments, a gene-editing agent can include zinc finger nucleases (ZFNs), transcription activator-like effector nucleases (TALENs), engineered meganucleases, or a clustered regularly interspaced short palindromic repeats (CRISPR) system. In some embodiments, a gene-editing agent can include RNA interference (e.g., short hairpin RNA (shRNA), small interfering RNA (siRNA), antisense oligonucleotide (ASO), or microRNA mimics). In some embodiments, the gene-editing agent can include CRISPR components. For example, in some embodiments, CRISPR components can include, but are not limited to, a guide RNA and a CRISPR-associated endonuclease (Cas protein). In some embodiments, the gene-editing agent can include a guide RNA (e.g., gRNA or sgRNA) and a CRISPR-associated endonuclease (Cas protein). In some embodiments, the gene-editing agent comprises shRNAs, siRNAs, ASOs, or microRNa mimics.

As used herein, the term “CRISPR” refers to a technique of sequence specific genetic manipulation relying on the clustered regularly interspaced short palindromic repeats pathway, which unlike RNA interference regulates gene expression at a transcriptional level. The term “gRNA” or “guide RNA” refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32 (12): 1262-7 and Graham, D., et al. Genome Biol. 2015; 16:260. The term “Single guide RNA” or “sgRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the single guide RNA can recognize a target RNA, for example, by hybridizing to the target RNA. In some embodiments, the single guide RNA comprises a sequence that is complementary to the target RNA. In some embodiments, the sgRNA can include one or more modified nucleotides. In some embodiments, the sgRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a single guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (IncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as cancers, neurodegeneration, cutaneous conditions, endocrine conditions, intestinal diseases, infectious conditions, neurological conditions, liver diseases, heart disorders, or autoimmune diseases. In some embodiments, a target RNA can be a therapeutic target for conditions such as cancers, neurodegeneration, cutaneous conditions, endocrine conditions, intestinal diseases, infectious conditions, neurological conditions, liver diseases, heart disorders, or autoimmune diseases.

In some embodiments, a method described herein can include assembling a modulation unit, wherein the modulation unit comprises an RNA binding protein (RBP), an exogenous RNA binding moicty, and a gene-editing agent. In some embodiments, the assembling of the modulation unit can be performed outside of a host cell. In some embodiments, the assembling can include plasmid construction.

In some embodiments, a method described herein can include delivering a modulation unit into a cell. In some embodiments, the delivering step comprises lipofection. In some embodiments, the delivering step comprises a virus-based delivery. In some embodiments, the virus-based delivery comprises adeno-associated virus or lentivirus.

In some embodiments, a method described herein can also include detecting change in a target RNA stability and/or translation, wherein a modulation unit modulates gene expression of the target RNA in a cell. As used herein, a “reporter mRNA” can refer to an mRNA that can be attached to another gene of interest, wherein the reporter mRNA can express a protein that is easily measured and identified and can be used as a marker to indicate whether the gene of interest in expressed in a cell or organism. In some embodiments, the detecting step comprises using a reporter mRNA. In some embodiments, a reporter mRNA can include a luciferase mRNA. In some embodiments, a reporter mRNA can include chloramphenicol acetyltransferase, β-galactosidase (GAL), β-glucuronidase, β-glucuronidase, firefly luciferase, Renilla luciferase, or green fluorescent protein (GFP).

### Methods of Identifying a Function of an RNA Binding Protein (RBP)

Provided herein are methods of identifying a function of an RNA binding protein (RBP) including (a) contacting the RBP to an exogenous RNA binding moiety; (b) allowing the exogenous RNA binding moiety to interact with an RNA structural motif; and (c) profiling the RBP tethered to the RNA structural motif, thereby identifying a function of the RBP.

In some embodiments, a function of an RNA binding protein can include regulating target RNA translation and/or stability. In some embodiments, a function of an RNA binding protein can include controlling global protein homeostasis by affecting levels of RNA translation regulators. In some embodiments, a function of an RNA binding protein can include RNA splicing, modulating RNA stability, RNA transport, or RNA translation. In some embodiments, a function of an RNA binding protein can include stabilizing a target RNA. In some embodiments, a function of an RNA binding protein can include destabilizing a target RNA. In some embodiments, a function of an RNA binding protein can include enhancing translation of a target RNA. In some embodiments, a function of an RNA binding protein can include suppressing translation of a target RNA.

In some embodiments, the contacting step can include an exogenous RNA binding moiety being fused to a RNA binding protein. In some embodiments, the exogenous RNA binding moiety can be fused to a RNA binding protein through an artificial RNA-protein interaction. In some embodiments, an exogenous RNA binding moiety can include a reporter mRNA. In some embodiments, an exogenous RNA binding moiety comprises a MS2 bacteriophage coat protein (MCP). In some embodiments, an RNA structural motif comprises a reporter mRNA. In some embodiments, the reporter mRNA comprises a MS2 genomic RNA stem-loop. As used herein, an “RNA structural motif” can refer to a collection of residues that fold into a stable three-dimensional (3D) structure of an RNA molecule. In some embodiments, an RNA structural motif can include an RNA hairpin loop, RNA internal loop, a tetraloop, a sarcin-ricin loop, or a T-loop. In some embodiments, an RNA structural motif can includes a MS2 genomic RNA stem-loop.

As used herein, “profiling” can refer to the measurement of an activity (e.g., expression) of one or more genes, to create a global picture of cellular function. In some embodiments, the profiling comprises transcriptome analysis or gene expression analysis. In some embodiments, the profiling comprises enhanced cross-linking immunoprecipitation (eCLIP). As used herein, “Enhanced crosslinking and immunoprecipitation (eCLIP)” refers to a method to profile RNAs bound by an RNA binding protein of interest. In some embodiments, eCLIP can be modified and used to profile RNAs bound by specific ribosomal subunit proteins. In some embodiments, enhanced crosslinking and immunoprecipitation (eCLIP) recovers protein-coding mRNAs (with a particular enrichment for coding sequence regions).

### EXAMPLES

The disclosure is further described in the following examples, which do not limit the scope of the disclosure.

### Example 1

### Generation of Resource of RBP Open-Reading Frames Fused to MS2 Coat Protein And Tethered Function Assays

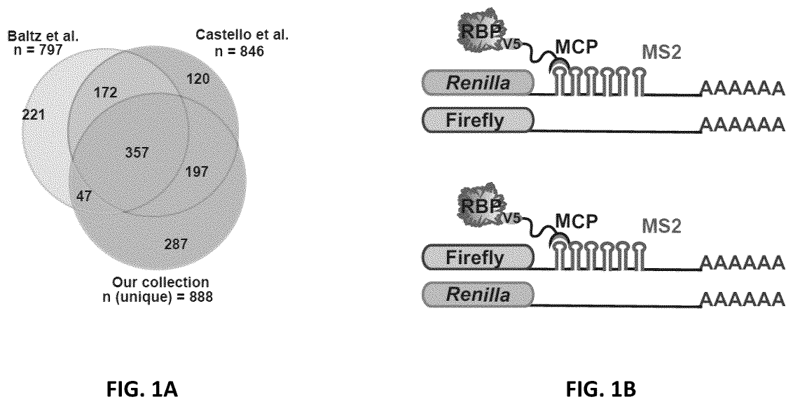

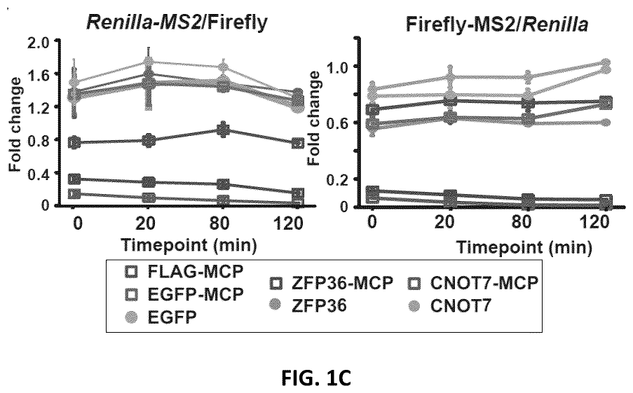

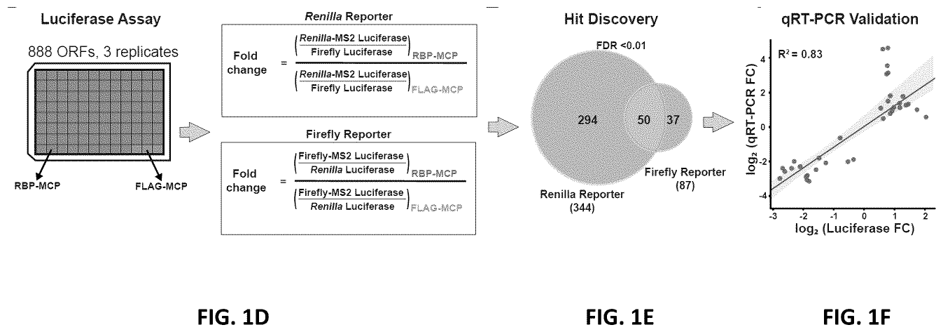

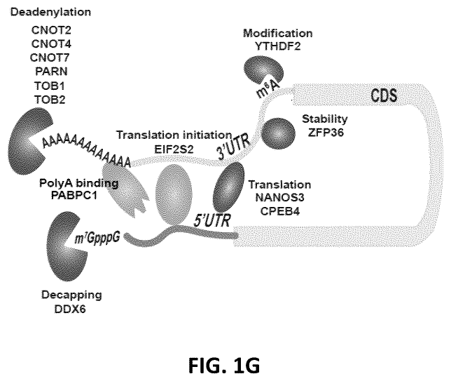

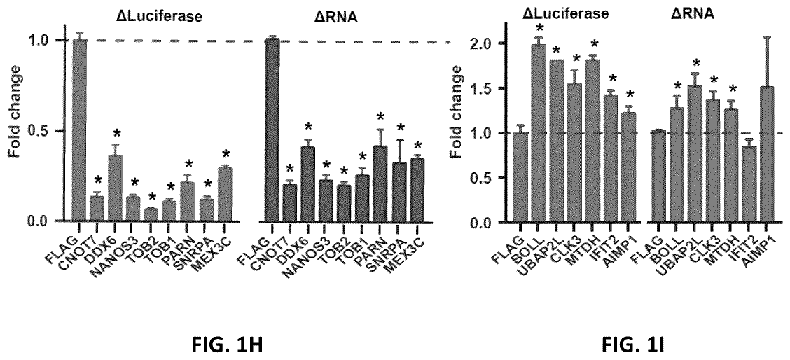

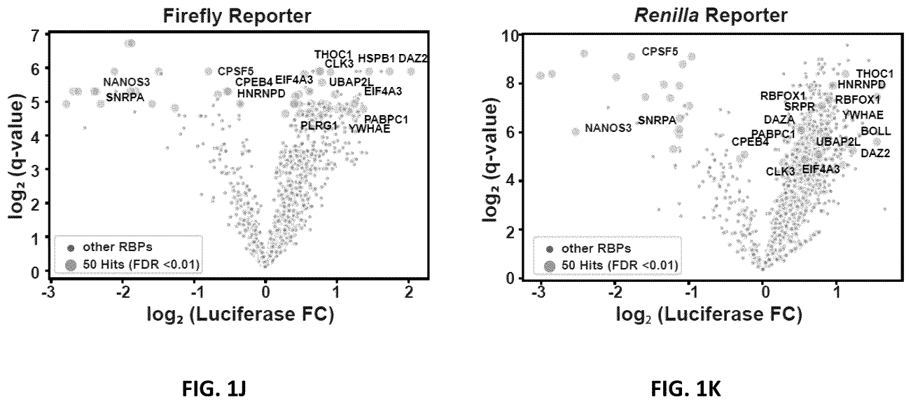

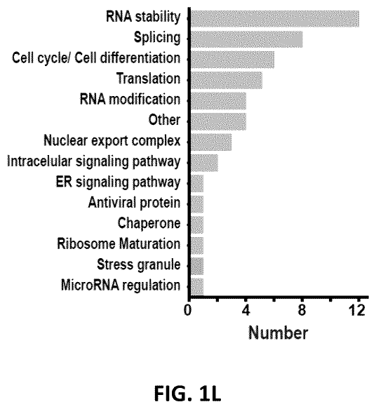

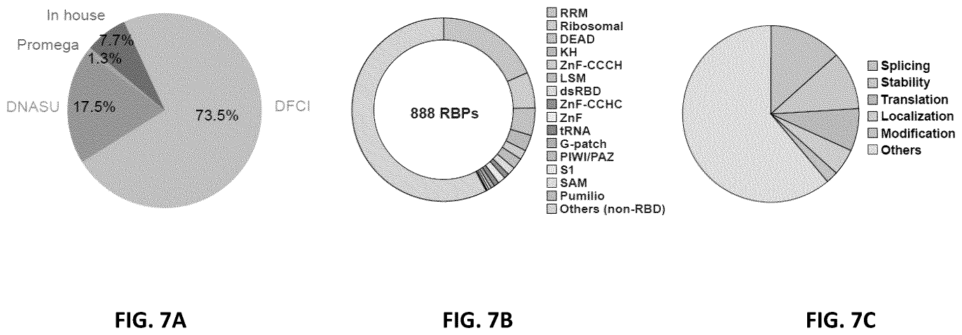

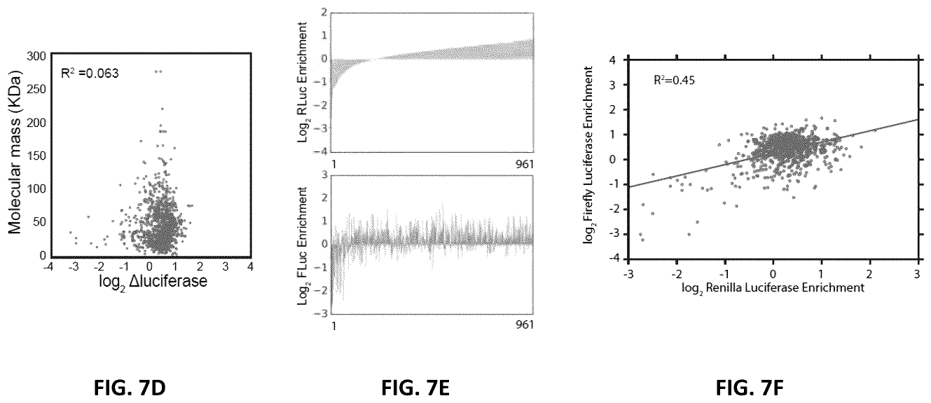

A collection of RBP expression constructs was assembled using in-house bioinformatics tools to extract genes annotated to contain RNA-binding domains as predicted by PFAM and PRINTS. This set was extended with mRNA-bound putative RBPs identified experimentally in two different studies which used UV-cross-linking and oligo (dT) capture followed by mass spectrometry. 888 unique RBPs with 1,062 RBP ORFs (FIG. 1A) were acquired from both commercial sources and through in-house cloning efforts (FIG. 7A; Table 1) and sub-cloned into two constructs using Gateway-mediated cloning: one that directs expression of the RBPs as fusion proteins with the V5 epitope tag C-terminally appended, and one with an additional bacteriophage MS2 coat protein (MCP) domain at the C-terminus. Overall, ˜40% of the 69 RBPs in the collection contain known canonical RNA-binding motifs, while the remainder may associate with RNA through other interaction domains or binding modes (FIG. 7B). Highlighting the need for assessing the roles of RBPs in RNA metabolism, Gene Ontology (GO) analysis showed that ˜60% of the RBPs in the collection have no known RNA-related functions (FIG. 7C). Thus, a comprehensive resource of representative ‘tethered’ and ‘untethered’ RBP expression libraries was assembled comprising the majority of all predicted and/or experimentally identified RBPs.

Next, a set of tetracycline-repressible luciferase reporter plasmids were constructed that measure the effect of RBP recruitment to the 3′UTR on reporter expression. F-Luc-6MS2 encodes firefly luciferase followed by 6 MS2 hairpin sequences inserted into the 3′UTR context of HBB (β-globin). To address potential reporter context dependencies, a corresponding Renilla luciferase construct was also generated. Matched constructs lacking MS2 sequences served as negative controls (FIG. 1B). To validate the system, each reporter was co-introduced into HeLa cells along with constructs expressing MCP-fused and unfused versions of ZFP36 (also known as Tristetraprolin, TTP), an RBP activator of AU-rich element (ARE)-mediated RNA decay, enhanced GFP (EGFP) or the FLAG peptide. As expected, ZFP36 but not enhanced GFP (EGFP) or the FLAG peptide, dramatically reduced protein levels of the luciferase reporter in a manner that depended on the presence of the tether but not the identity of the luciferase protein (FIG. 1C). This demonstrated that tethered ZFP36 can recruit functional CCR4-NOT deadenylase complexes, which contain the Caf1 subunit CNOT7 (an RNase), to the reporter. Tethering of CNOT7 itself recapitulated this finding, indicating that productive recruitment is not limited to sequence-specific RBPs (such as ZFP36), but extends to effector RBPs (such as CNOT7) (FIG. 1C).

TABLE 1

| Gene symbol | Accession number | MW (kDa) | GO Term | Group | Domain | Source |
|---|---|---|---|---|---|---|
| A1CF | BC054873.1 | 13.8 | Modification | | RRM | DFCI |
| ABT1 | BC048812.1 | 31.1 | Other | Baltz/Castello | RRM | DFCI |
| ABT1 | BC066313.1 | 31.1 | Other | Baltz/Castello | RRM | DFCI |
| ACAA2 | BC001918.1 | 41.9 | Other | | Other | DFCI |
| ACOT9 | | 48.9 | Other | | Other | In-house |
| ACTN4 | BC005033.1 | 104.9 | Other | Castesllo | Other | DFCI |
| ADAD1 | BC040229 | 62.8 | Other | | dsRBD | In-house |
| ADAD2 | BC033491.1 | 61.8 | Other | | dsRBD | DFCI |
| ADAR | BC038227 | 136.0 | Modification | Baltz/Castello | dsRBD | DNASU |
| ADARB1 | BC065545.1 | 76.6 | Modification | Baltz | dsRBD | DFCI |
| ADD1 | BC013393 | 44.0 | Other | | Other | DNASU |
| ADK | BC003568.1 | 38.7 | Other | Castello | Other | DFCI |
| AGGF1 | BC002828.2 | 12.5 | Other | | Other | DFCI |
| AGGF1 | BC032844.1 | 80.9 | Other | | Other | DFCI |
| AHNAK | BC012477.1 | 16.1 | Other | Castello | Other | DFCI |
| AHNAK | BC000926.1 | 16.2 | Other | Castello | Other | DFCI |
| AIMP1 | BC014051.2 | 34.4 | Translation | | tRNA | DFCI |
| AK8 | BC034776.1 | 54.9 | Other | | Ostler | DFCI |
| | BC050576.1 | | | | | |
| ALDH18A1 | HQ268499 | 87.3 | Other | Castello | Other | DNASU |
| ALDH6A1 | BC004909.1, | 57.8 | Other | Castello | Other | DFCI |
| | BC032371.1 | | | | | |
| ANKHD1 | BC040231.1 | 91.5 | Other | Baltz/Castello | KH | DFCI |
| ANKHD1 | BC004457 | 46.1 | Other | Baltz/Castello | KH | In-house |
| ANXA2 | BC052567.1 | 38.6 | Other | Castello | Other | DFCI |
| ANXA2 | BC009564.1 | 38.6 | Other | Castello | Other | DFCI |
| ANXA2 | BC023990.1 | 38.6 | Other | Castello | Other | DFCI |
| APEH | BC000362.2 | 81.2 | Other | Castello | Other | DFCI |
| APOBEC3A | BC126416.1 | 23.0 | Other | | Other | DFCI |
| APOBEC3B | BC053859.1 | 29.8 | Other | Baltz | Other | DFCI |
| APOBEC3C | BC011739.2 | 22.8 | Other | Baltz/Castello | Other | DFCI |
| APOBEC3B | BC017022.1 | 46.6 | Other | | Other | DFCI |
| APOBEC3F | BC038808.1 | 45.0 | Other | Baltz | Other | DFCI |
| APOBEC3F | BC061914 | 9.4 | Other | Baltz | Other | In-house |
| APOBEC3G | BC024268.1 | 46.4 | Other | | Other | DFCI |
| APOBEC3H | BC069023.1 | 21.5 | Other | | Other | DFCI |
| APOBEC4 | BC021711 | 41.6 | Other | | Other | In-house |
| ARL6IP4 | uc004dat.1 | 24.0 | Splicing | Baltz/Castello | Other | DFCI |
| ARL6IP4 | BC001958.1 | 24.6 | Splicing | Baltz/Castello | Other | DFCI |
| ASCC1 | BC012291.1 | 41.2 | Other | | KH | DFCI |
| ASCC3 | BC050681.1 | 13.0 | Other | Baltz | Other | DFCI |
| ASS1 | BC021676.1 | 46.5 | Other | Castello | Other | DFCI |
| ASS1 | BC009243.2 | 46.5 | Other | Castello | Other | DFCI |
| ATP5C1 | BC000470.2, | 33.0 | Other | Castello | Other | DFCI |
| | BC000931.3 | | | | | |
| ATF5C1 | BC016812.1 | 33.0 | Other | Castello | Other | DFCI |
| ATXN1 | BC117125 | 86.9 | Localization | | Other | In-house |
| BCCIP | BC009771.1 | 36.1 | Other | Castello | Other | DFCI |
| BCDIN3D | BC053560.1 | 33.2 | Modification | | Other | DFCI |
| BCL7B | BC000956 | 22.2 | Other | | Other | In-house |
| BMS1 | BC043345.1 | 145.8 | Other | Baltz/Castello | Other | DFCI |
| BOLL | BC033874.1 | 31.3 | Translation | | RRM | DFCI |
| BST2 | BC033873.1 | 19.8 | Other | Castello | Other | DFCI |
| BTG1 | BC016759 | 19.2 | Other | | Other | DNASU |
| BTG2 | BC105949 | 17.4 | Other | | Other | DNASU |
| BTG3 | BC011957.1 | 34.2 | Other | | Other | DNASU |
| BTG4 | BC031045 | 24.0 | Other | | Other | DNASU |
| BTN3A3 | BC015815.2 | 65.0 | Other | | Other | DFCI |
| BUD13 | BC006350.2 | 70.5 | Localization | Castello | Other | DFCI |
| BYSL | BC007340.2 | 37.1 | Other | Castello | Other | DFCI |
| BZW1 | BC026303.1 | 48.0 | Other | Castello | Other | DFCI |
| C16ORF88 | BC117562.1 | 51.6 | Other | Castello | Other | DFCI |
| C1D | BC005235.1 | 16.0 | Other | | Other | DFCI |
| C1D | BC009584.1, | 16.0 | Other | | Other | DFCI |
| | BC0095891, | | | | | |
| | BC016284.2 | | | | | |
| C1ORF131 | BC036800.1 | 31.4 | Other | Baltz/Castello | Other | DFCI |
| C1ORF35 | BC002843.2 | 29.4 | Other | Baltz/Castello | Other | DFCI |
| C9ORF72 | C9ORF72 | 54.3 | Other | | Other | In-house |
| CALR | BC002500.2, | 48.1 | Other | Castello | Other | DFCI |
| | BC007911.1, | | | | | |
| | BC020493.1 | | | | | |
| CCDC137 | BC009369.2 | 33.2 | Other | Baltz/Castello | Other | DFCI |
| CCDC59 | BC020647.1 | 28.7 | Other | Castello | Other | DFCI |
| CCDC75 | BC071798.1 | 18.6 | Other | | Other | DFCI |
| CCDC9 | BC002787.2, | 59.7 | Other | Baltz/Castello | Other | DFCI |
| | BC009743.2 | | | | | |
| CCNL1 | JF432881 | 59.6 | Other | | Other | DNASU |
| CCNL2 | BC016333.1 | 24.6 | Other | | Other | DNASU |
| CCT4 | BC106934.1 | 57.9 | Other | Castello | Other | DFCI |
| CCT6A | BC106942.1 | 58.0 | Other | Castello | Other | DFCI |
| CDC2L5 | NM_003718.3 | 164.9 | Other | | Other | DNASU |
| CDC40 | HQ258473 | 65.5 | Localization | Castello | Other | DNASU |
| CDC42EP4 | BC010451.1 | 38.0 | Other | | Other | DFCI |
| CDC42EP4 | BC002774.1 | 38.0 | Other | | Other | DFCI |
| CELF3 | BC052491.1 | 50.5 | Splicing | | RRM | DFCI |
| CELF4 | BC001946.1 | 51.8 | Translation | | RRM | DFCI |
| CELF5 | BC028101.1 | 52.4 | Other | | RRM | DFCI |
| CHAF1B | | 61.5 | Other | | Other | DNASU |
| CHAF1B | JF432525 | 61.5 | Other | | Other | DNASU |
| CHD2 | BC007347.2 | 56.8 | Other | Baltz/Castello | Other | DFCI |
| CHTOP | BC120961.1 | 26.5 | Localization | Baltz | Other | DFCI |
| CIR1 | BC015040.1 | 23.3 | Other | | Other | DNASU |
| CIRBP | BC000901.1 | 18.6 | Stability | Baltz/Castello | RRM | DFCI |
| CIRBP | BC000403.2 | 18.6 | Stability | Baltz/Castello | RRM | DFCI |
| CIRH1A | BC009348.2 | 76.9 | Other | Castello | Other | DFCI |
| CISD2 | BC032300.1 | 15.3 | Other | Castello | Other | DFCI |
| CLASRP | | 77.2 | Splicing | | Other | DNASU |
| CLK1 | BC031549.1 | 57.3 | Splicing | | Other | DNASU |
| CLK2 | BC014067.2 | 60.0 | Splicing | | Other | DNASU |
| CLK3 | BC019881.1 | 58.6 | Splicing | Baltz/Castello | Other | DFCI |
| CLK3 | BC002555.2 | 58.6 | Splicing | Baltz/Castello | Other | DFCI |
| CLP1 | BC000446.1 | 47.6 | Splicing | | Other | DNASU |
| CMSS1 | BC006475.1 | 31.8 | Other | | Other | DFCI |
| CMBP | BC000288.2 | 18.7 | Other | Baltz/Castello | zf-CCHC | DFCI |
| CNBP | BC014911.1 | 18.7 | Other | Baltz/Castello | zf-CCHC | DFCI |
| CNOT1 | | | Stability | Baltz/Castello | Other | In-house |
| CNOT10 | BC002928.2 | 79.4 | Stability | | Other | DNASU |
| CNOT10 | BC002931.2 | 82.3 | Stability | | Other | DNASU |
| CNOT2 | BC011826 | 59.7 | Stability | | Other | DNASU |
| CNOT3 | BC016474 | 81.9 | Stability | | Other | DNASU |
| CNOT4 | BC035590.1 | 63.1 | Stability | Baltz | RRM | DFCI |
| CNOT6 | NM_015455.3 | 63.3 | Stability | | Other | DNASU |
| CNOT6 | BC150174 | 10.3 | Stability | | Other | DNASU |
| CNOT6L | NM_144571 | 63.0 | Stability | | Other | DNASU |
| CNOT6L | BC152816 | 63.0 | Stability | | Other | DNASU |
| CNOT7 | BC007315.2 | 28.4 | Stability | | Other | DFCI |
| CNOT7 | BC060852.1 | 32.7 | Stability | | Other | DFCI |
| CNOT8 | BC017366.2 | 33.5 | Stability | | Other | DFCI |
| COA6 | BC116455.1 | 14.1 | Other | | Other | DFCI |
| CORO1A | BC126385.1, | 51.0 | Other | | Other | DFCI |
| | BC126387.1 | | | | | |
| CPEB1 | BC036348.1 | 53.6 | Translation | | RRM | DNASU |
| CPEB2 | BC103939.1 | 61.3 | Translation | Baltz/Castello | RRM | DFCI |
| CPEB4 | BC036899.1 | 36.2 | Other | Baltz/Castello | RRM | DFCI |
| CPEB4 | BC117150 | 80.2 | Other | Baltz/Castello | RRM | In-house |
| CPNE3 | BC015734.1 | 5.0 | Other | Castello | Other | DFCI |
| CPSF1 | BC017232 | 160.9 | Localization | | Other | DNASU |
| CPSF2 | BC070095.1 | 88.5 | Localization | | Other | DNASU |
| CPSF3 | BC011654 | 77.4 | Localization | | Other | DNASU |
| CPSF3L | AM393218 | 44.1 | Other | | Other | DNASU |
| CPSF4 | BC050738.1 | 27.4 | Modification | Baltz | ZnF-CCCH | DNASU |
| CPSF4 | BC003101.1 | 27.5 | Modification | Baltz | ZnF-CCCH | DNASU |
| CPSF4L | BC157870.1 | 20.7 | Other | | ZnF-CCCH | DNASU |
| CPSF5 | BC001403 | 26.2 | Other | | Other | DNASU |
| CPSF5 | BX537360 | 26.2 | Other | | Other | DNASU |
| CPSF6 | BC005000.1 | 52.3 | Other | Baltz/Castello | RRM | DFCI |
| CPSF6 | BC000714.2 | 63.5 | Other | Baltz/Castello | RRM | DFCI |
| CPSF7 | BC018135.1 | 52.0 | Splicing | Baltz/Castello | RRM | DFCI |
| CRABP2 | | 15.6 | Other | | Other | DNASU |
| CRKRS | NM_016507 | 164.2 | Other | | Other | DNASU |
| CSNK1G2 | | 47.5 | Other | | Other | In-house |
| CSTF1 | BC001011 | 48.4 | Splicing | Baltz/Castello | Other | DNASU |
| CSTF2 | BC017712 | 61.0 | Splicing | Baltz/Castello | RRM | DNASU |
| CSTF2T | BC028239.1 | 64.5 | Other | Baltz/Castello | RRM | DFCI |
| CSTF3 | HQ447685 | 12.1 | Splicing | Baltz | Other | DNASU |
| CTNNA1 | BC031262.1 | 59.5 | Other | Castello | Other | DFCI |
| CUGBP1 | BC031079 | 51.6 | Other | | Other | DNASU |
| CWC15 | BC040946.1 | 26.6 | Splicing | Castello | Other | DFCI |
| DARS | BC000629.2 | 57.1 | Translation | Castello | tRNA | DFCI |
| DAZ2 | BC113006 | 60.4 | Other | | RRM | In-house |
| DAZ3 | BC113005.1 | 49.5 | Other | | RRM | DFCI |
| DAZ4 | BC047480.1, | 44.1 | Other | | RRM | DFCI |
| | BC047617.1 | | | | | |
| DAZAP1 | BC012062.1 | 43.4 | Other | Baltz/Castello | RPM | DFCI |
| DBR1 | BC009472.1 | 61.6 | Splicing | Castello | Other | DFCI |
| DCD | BC062682.1 | 11.3 | Other | | Other | DFCI |
| DCN | BC005322.1 | 39.7 | Other | | Other | DFCI |
| DCP1A | BC007439.2 | 63.3 | Other | | Other | DNASU |
| DCP2 | BC064593.1 | 44.4 | Stability | | Other | DNASU |
| DDX1 | BC012132.1 | 82.4 | Translation | Baltz/Castello | DEAD | DFCI |
| DDX1 | BC012739.2 | 77.9 | Translation | Baltz/Castello | DEAD | DFCI |
| DDX11 | BC011264.1 | 108.3 | Other | | Other | DNASU |
| DDX11 | BC111733.1 | 101.6 | Other | | Other | DNASU |
| DDX17 | BC000595.2 | 72.4 | Other | Baltz/Castello | DEAD | DFCI |
| DDX18 | BC001238.1, | 75.4 | Other | Baltz/Castello | DEAD | DFCI |
| | BC003360.1, | | | | | |
| | BC024739.1 | | | | | |
| DDX19A | BC005162.2, | 54.0 | Localization | | DEAD | DFCI |
| | BC006544.2 | | | | | |
| DDX19B | BC010008.2 | 41.8 | Localization | | DEAD | DFCI |
| DDX19B | BC003626.2 | 53.9 | Localization | | DEAD | DFCI |
| DDX20 | BC031062.1 | 92.2 | Other | | DEAD | DFCI |
| DDX20 | BC011566 | 92.2 | Other | | DEAD | In-house |
| DDX21 | BC004182 | 79.7 | Other | Baltz/Castello | DEAD | In-house |
| DDX21 | BC008071.2 | 87.3 | Other | Baltz/Castello | DEAD | DNASU |
| DDX23 | BC002366.2 | 95.6 | Splicing | Baltz | DEAD | DFCI |
| DDX24 | BC008847 | 96.3 | Other | Baltz/Castello | DEAD | DNASU |
| DDX27 | HQ253546 | 86.6 | Other | Baltz/Castello | DEAD | DNASU |
| DDX27 | HQ258508 | 89.6 | Other | Baltz/Castello | DEAD | DNASU |
| DDX28 | BC024273.1 | 59.6 | Other | Baltz/Castello | DEAD | DFCI |
| DDX31 | BC012726 | 75.5 | Other | Baltz/Castello | DEAD | In-house |
| DDX31 | BC158832 | 64.1 | Other | Baltz/Castello | DEAD | DNASU |
| | (NM_138620.1) | | | | | |
| DPX39A | BC032128.2 | 36.6 | Localization | Castello | DEAD | DFCI |
| DDX39A | BC001009.2 | 49.1 | Localization | Castello | DEAD | DFCI |
| DDX39A | BC010455.2 | 35.1 | Localization | Castello | DEAD | DFCI |
| DDX41 | BC015476.1 | 69.8 | Splicing | Baltz/Castello | DEAD | DFCI |
| DDX43 | BC066938.1 | 72.9 | Other | | KH | DFCI |
| DDX47 | BC009379.2 | 36.7 | Splicing | Baltz/Castello | DEAD | DFCI |
| DDX49 | BC002674.2 | 54.2 | Other | Castello | DEAD | DFCI |
| DDX5 | BC016027 | 69.1 | Splicing | Baltz/Castello | DEAD | DNASU |
| DDX50 | BC000272.1 | 82.6 | Other | Baltz/Castello | DEAD | DFCI |
| DDX51 | notBC040185 | 11.9 | Other | Baltz/Castello | DEAD | In-house |
| DDX52 | BC041785.1 | 67.5 | Other | Baltz/Castello | DEAD | DFCI |
| DDX53 | BC067878.1 | 71.2 | Other | | KH | DFCI |
| DDX54 | BC001132.2 | 25.5 | Other | Baltz/Castello | DEAD | DFCI |
| DDX55 | BC035911.1 | 24.3 | Other | Castello | DEAD | DFCI |
| DDX55 | BC030020.2 | 68.5 | Other | Castello | DEAD | DFCI |
| DDX56 | BC001235 | 61.7 | Other | Castello | DEAD | DNASU |
| DDX59 | BC041801.1 | 68.9 | Other | | DEAD | DFCI |
| DDX6 | BC085007.1 | 54.4 | Stability | Baltz/Castello | DEAD | DFCI |
| DDX60 | BC020601.1 | 20.8 | Other | | DEAD | DFCI |
| DGCR8 | BC009323.2 | 55.6 | Other | | dsRSD | DFCI |
| DHX18 | BC008825.2, | 119.4 | Splicing | Baltz/Castello | DEAD | DFCI |
| | BC009392.2 | | | | | |
| DHX29 | BC056219.1 | 155.3 | Translation | Baltz | DEAD | DFCI |
| DHX30 | BC014237.2 | 56.5 | Other | Baltz/Castello | dsRBD | DFCI |
| DHX32 | DQ895902 | 84.4 | Other | | Other | DNASU |
| DHX33 | BC042040.1 | 54.6 | Other | Castello | DEAD | DFCI |
| DHX34 | BC172389 | 128.1 | Other | Baltz | DEAD | DNASU |
| DHX35 | HQ258480 | 78.9 | Splicing | | DEAD | DNASU |
| DHX36 | BC036035.1 | 111.5 | Other | Baltz/Castello | DEAD | DFCI |
| DHX37 | BC004463.1 | 3.9 | Other | | DEAD | DNASU |
| DHX38 | BC004235 | 140.5 | Localization | Baltz | DEAD | DNASU |
| DHX40 | BC024187.2 | 88.6 | Other | | DEAD | DNASU |
| DHX57 | BC060778.1 | 63.2 | Other | Baltz/Castello | DEAD | DFCI |
| DHX58 | BC014949 | 76.6 | Other | | Other | In-house |
| DHX58 | JF432115 | 76.6 | Other | | Other | DNASU |
| DHX8 | BC044586.1 | 138.8 | Splicing | Baltz/Castello | DEAD | DFCI |
| DIAPH1 | BC007411 | 141.4 | Other | Castello | Other | DNASU |
| DICER1 | BC150287.1 | 218.7 | Other | | PIWI/PAZ | DFCI |
| DIEXF | BC022964.1 | 87.1 | Other | Castello | Other | DFCI |
| DIMT1 | BC010874.2 | 35.2 | Modification | | Other | DFCI |
| DIS3 | BC038101.1 | 30.8 | Stability | | Other | DNASU |
| DIS3L | BC022089.2 | 111.1 | Other | | Other | DNASU |
| DKC1 | BC010015.2 | 57.7 | Modification | Castello | Other | DFCI |
| DMGDH | BC156312 | 96.8 | Other | Castello | Other | DNASU |
| DMPK | BC062553 | 69.4 | Other | | Other | DNASU |
| DNAJC17 | BC000048.2 | 34.7 | Other | | RRM | DFCI |
| DNAJC2 | BC056682.1 | 19.0 | Other | Castello | Other | DFCI |
| DNAJC5 | BC053642.1 | 22.1 | Other | | Other | DFCI |
| DND1 | BC033496.1 | 38.7 | Other | | RRM | DFCI |
| DNTTIP2 | BC130622.1 | 84.5 | Other | Baltz/Castello | Other | DFCI |
| DPPA5 | BC137549.1 | 13.5 | Other | | Other | DFCI |
| DUS2L | BC006527.2 | 55.0 | Other | | Other | DFCI |
| DUT | BC033645.1 | 17.7 | Other | Castello | Other | DFCI |
| DYNC1H1 | BC064521.1 | 22.2 | Other | Baltz/Castello | Other | DFCI |
| DYNC1LI1 | BC131620.1 | 58.6 | Other | Castello | Other | DFCI |
| DZIP3 | BC083882.1 | 138.6 | Other | Castello | Zn-Finger | DFCI |
| EBNA1BP2 | BC009175.2 | 34.9 | Other | Baltz/Castello | Other | DFCI |
| EDC3 | BC011534.1, | 56.1 | Stability | | LSM | DNASU |
| | BC021271.2 | | | | | |
| EDF1 | BC015500.1 | 16.4 | Other | Baltz/Castello | Other | DFCI |
| EEF1A1 | BC008557.1, | 50.1 | Translation | Baltz/Castello | Other | DFCI |
| | BC009733.1, | | | | | |
| | BC009875.2, | | | | | |
| | BC010735.1, | | | | | |
| | BC012891.1, | | | | | |
| | BC014224.2, | | | | | |
| | BC018150.1, | | | | | |
| | BC018641.2, | | | | | |
| | BC010669.1, | | | | | |
| | BC021686.1, | | | | | |
| | BC028674.1, | | | | | |
| | BC038339.1, | | | | | |
| | BC066893.1 | | | | | |
| EEF1A1 | notBC131708 | 50.1 | Translation | Baltz/Castello | Other | In-house |
| EEF2 | BC126259.1 | 95.3 | Translation | Baltz/Castello | Other | DFCI |
| EFTUD2 | BC002360.2 | 109.5 | Splicing | Baltz/Castello | Other | DFCI |
| EIF2AK2 | BC101475.1 | 62.1 | Translation | Baltz | dsRBD | DFCI |
| EIF2C1 | BC083275.1 | 97.2 | Other | Baltz | Other | DFCI |
| EIF2C2 | BC007633.1 | 42.4 | Other | Baltz/Castello | Other | DFCI |
| EIF2C2 | BC018727.1 | 66.3 | Other | Baltz/Castello | Other | DFCI |
| EIF2C3 | BC066888.1 | 25.6 | Other | Baltz/Castello | Other | DFCI |
| EIF2S2 | BC000934.2 | 38.4 | Translation | Baltz/Castello | Other | DFCI |
| EIF2S2 | BC000461.2 | 38.4 | Translation | Baltz/Castello | Other | DFCI |
| EIF3C | BC001571.1 | 105.3 | Translation | Baltz/Castello | Other | DFCI |
| EIF3D | BC080515.1 | 64.0 | Translation | Baltz/Castello | Other | DFCI |
| EIF3G | BC000733.2 | 35.6 | Translation | Baltz/Castello | RRM | DFCI |
| EIF3H | BC000386.2 | 39.9 | Translation | Castello | Other | DFCI |
| EIF3L | BC001101.2, | 66.7 | Translation | Baltz/Castello | Other | DFCI |
| | BC007510.2 | | | | | |
| EIF4A1 | BC009585.1 | 46.2 | Stability | Baltz/Castello | DEAD | DFCI |
| EIF4A2 | BC015842.1 | 46.4 | Stability | Baltz/Castello | DEAD | DFCI |
| EIF4A2 | BC012547 | 46.5 | Stability | Baltz/Castello | DEAD | In-house |
| EIF4A2 | BC016295 | 21.0 | Stability | Baltz/Castello | DEAD | In-house |
| EIF4A2 | BC048105 | 46.5 | Stability | Baltz/Castello | DEAD | In-house |
| EIF4A3 | BC004386.1, | 46.9 | Stability | Baltz/Castello | DEAD | DFCI |
| | BC011151.1 | | | | | |
| EIF4A3 | BC003662 | 46.9 | Stability | Baltz/Castello | DEAD | In-house |
| EIF48 | BC073139.1 | 69.2 | Stability | Baltz/Castello | RRM | DFCI |
| EIF4H | BC021214.2, | 25.2 | Translation | Baltz/Castello | RRM | DFCI |
| | BC066928.1 | | | | | |
| EIF5B | BC032639.1 | 138.8 | Translation | Baltz/Castello | Other | DFCI |
| ELAC2 | BC001939.1 | 92.2 | Other | Baltz/Castello | Other | DFCI |
| ELAVL1 | BC003376 | 36.1 | Stability | Baltz/Castello | RRM | In-house |
| ELAVL2 | BC030692.1 | 38.0 | Other | Baltz/Castello | RRM | DFCI |
| ELAVL3 | BC014144 | 39.5 | Other | | RRM | In-house |
| ELAVL4 | BC036071.1 | 40.4 | Other | | RRM | DFCI |
| ELMOD3 | BC001942.1 | 44.3 | Other | | Other | DNASU |
| EMG1 | BC055314.1 | 26.7 | Other | Baltz/Castello | Other | DFCI |
| ENOX1 | BC024178 | 73.3 | Other | | RRM | In-house |
| ENOX2 | BC019254.1 | 36.9 | Other | | RRM | DFCI |
| ERAL1 | BC019094.2 | 48.3 | Other | Baltz/Castello | KH | DFCI |
| ERCC3 | BC008820.2 | 89.3 | Other | | Other | DFCI |
| ERI3 | BC001072.2 | 14.5 | Other | Baltz/Castello | Other | DFCI |
| ESRP1 | BC067098.1 | 75.6 | Splicing | | RRM | DFCI |
| EWSR1 | BC000527 | 37.8 | Other | Baltz/Castello | RRM | In-house |
| EWSR1 | BC004817 | 68.5 | Other | Baltz/Castello | RRM | In-house |
| EWSR1 | BC011048 | 68.4 | Other | Baltz/Castello | RRM | In-house |
| EWSR1 | BC072442 | 68.4 | Other | Baltz/Castello | RRM | In-house |
| EXOSC1 | BC022067.2 | 21.5 | Stability | | Other | DNASU |
| EXOSC10 | BC073788.1 | 100.8 | Stability | Baltz/Castello | Other | DNASU |
| EXOSC2 | BC000747 | 32.9 | Stability | | Other | DNASU |
| EXOSC3 | BC008880.2 | 29.6 | Stability | | Other | DNASU |
| EXOSC3 | BC002437.2 | 29.6 | Stability | | Other | DNASU |
| EXOSC4 | BC002777.2 | 26.4 | Stability | | Other | DNASU |
| EXOSC5 | BC007742.2 | 25.3 | | | Other | DNASU |
| EXOSC7 | BC012831.2 | 31.8 | Stability | | Other | DNASU |
| EXOSC8 | BC020773.1 | 30.0 | Stability | | Other | DNASU |
| EXOSC9 | | 48.9 | Stability | Castello | Other | DNASU |
| EZR | BC013903.2 | 89.4 | Other | Baltz/Castello | Other | DFCI |
| FAM120A | BC111736.1 | 121.9 | Other | Baltz/Castello | Other | DFCI |
| FAM120A | BC098584.1 | 121.9 | Other | Baltz/Castello | Other | DFCI |
| FAM208A | BC129986.1 | 125.4 | Other | | Other | DFCI |
| FAM32A | BC090639.1, | 13.2 | Other | Castello | Other | DFCI |
| | BC017286.1 | | | | | |
| FAM46A | BC000683.2 | 49.7 | Other | Castello | Other | DFCI |
| FAM98A | CCSB53266.1 | 55.3 | Other | Baltz/Castello | Other | DFCI |
| FAM98A | BC060860.1 | 55.2 | Other | Baltz/Castello | Other | DFCI |
| FANCM | BC036056.1 | 75.6 | Other | | Other | DFCI |
| FASN | BC007909.1 | 48.3 | Other | Baltz/Castello | Other | DFCI |
| FASTK | BC011770.2 | 61.1 | Splicing | | Other | DFCI |
| FASTKD1 | BC032687.2 | 77.2 | Other | Baltz/Castello | Other | DFCI |
| FASTKD2 | BC001544.1 | 81.5 | Other | Baltz/Castello | Other | DFCI |
| FASTKD3 | BC113563.1 | 75.7 | Other | Castello | Other | DFCI |
| FASTKD5 | BC007413.2 | 88.8 | Other | Baltz | Other | DFCI |
| FBL | BC019260.1 | 33.8 | Modification | Baltz/Castello | Other | DFCI |
| FCF1 | BC080600.1 | 12.8 | Other | Castello | Other | DFCI |
| FDPS | BC010004.2 | 48.3 | Other | Castello | Other | DFCI |
| FGF17 | BC113489.1 | 24.9 | Other | | Other | DFCI |
| FGF17 | | 24.9 | Other | | Other | DNASU |
| FGF19 | BC017664.1 | 24.0 | Other | | Other | DFCI |
| FGF19 | JF432499 | 24.0 | Other | | Other | DNASU |
| FIP1L1 | AL136310 | 58.4 | Other | Baltz/Castello | Other | DNASU |
| FKBP3 | BC016288.1, | 25.2 | Other | Castello | Other | DFCI |
| | BC020809.1 | | | | | |
| FKBP4 | BC001786.1, | 51.8 | Other | Castello | Other | DFCI |
| | BC007924.2 | | | | | |
| FLYWCH2 | BC014089.2 | 14.6 | Other | Castello | Other | DFCI |
| FMR1 | BC038998 | 34.1 | Localization | Baltz/Castello | KH | In-house |
| FNDC3B | BC012204.1 | 7.3 | Other | Castello | Other | DFCI |
| FRG1 | BC053397.1 | 29.2 | Splicing | Castello | Other | DFCI |
| FSCN1 | BC000521.2, | 54.5 | Other | Castello | Other | DFCI |
| | BC007348.2 | | | | | |
| FTO | NM_001080432 | 58.3 | Modification | | Other | DNASU |
| FTSJ3 | BC000131.1 | 65.7 | Other | Baltz/Castello | Other | DFCI |
| FUBP1 | BC017247 | 68.7 | Other | Baltz/Castello | KH | DNASU |
| FUBP3 | BC007874.2 | 28.5 | Other | Baltz/Castello | KH | DFCI |
| FUS | BC000402.2, | 53.4 | Splicing | Baltz/Castello | RRM | DFCI |
| | BC082459.1 | | | | | |
| FUSIP1 | BC010074 | 21.0 | Other | | Other | In-house |
| FXR2 | BC020090.1 | 74.2 | Other | Baltz/Castello | KH | DFCI |
| FXR2 | BC051907.1 | 74.2 | Other | Baltz/Castello | KH | DFCI |
| FZD10 | BC074997.2 | 66.3 | Other | | Other | DFCI |
| FZD3 | NM_017412 | 76.3 | Other | | Other | DNASU |
| FZD4 | BC114527.1 | 59.9 | Other | | Other | DFCI |
| FZD4 | | 59.9 | Other | | Other | DNASU |
| FZD7 | BC015915.1 | 63.8 | Other | | Other | DFCI |
| FZD8 | BC111845 | 73.3 | Other | | Other | DNASU |
| FZD9 | BC026333 | 64.5 | Other | | Other | DNASU |
| G3BP1 | BC000997.1 | 52.2 | Other | Baltz/Castello | RRM | DFCI |
| G3BP2 | BC011731.2 | 50.8 | Localization | Baltz/Castello | RRM | DFCI |
| GANAB | BC065266.1 | 96.2 | Other | Castello | Other | DFCI |
| GAFDH | BC001601.1, | 36.1 | Other | | Other | DNASU |
| | BC004109.2, | | | | | |
| | BC009081.1, | | | | | |
| | BC013310.2, | | | | | |
| | BC023632.2, | | | | | |
| | BC025925.1, | | | | | |
| | BC026907.1, | | | | | |
| | BC029618.1 | | | | | |
| GAR1 | BC003413.1 | 22.3 | Other | Baltz/Castello | Other | DFCI |
| GFM1 | BC049210.1 | 83.5 | Translation | Castello | Other | DFCI |
| GLE1 | BC030012.1 | 79.9 | Localization | | Other | DNASU |
| GLRX3 | BC014372.1 | 21.5 | Other | Castello | Other | DFCI |
| GLRX3 | BC005289.1 | 37.4 | Other | Castello | Other | DFCI |
| GLTSCR2 | BC006311.2, | 54.4 | Other | Baltz/Castello | Other | DFCI |
| | BC010095.2 | | | | | |
| GNB2L1 | BC014788.1 | 35.1 | Other | Baltz/Castello | Other | DFCI |
| GNL2 | BC000107.2 | 83.7 | Other | Baltz/Castello | Other | DFCI |
| GNL2 | BC009250.2 | 83.7 | Other | Baltz/Castello | Other | DFCI |
| GNL3 | BC001024.2 | 62.0 | Other | Baltz/Castello | Other | DFCI |
| GNL3L | BC011720.2 | 65.6 | Other | Castello | Other | DFCI |
| GPANK1 | BC008783.1 | 39.3 | Other | | Other | DFCI |
| GPATCH2 | BC063474.1 | 42.6 | Other | | Other | DFCI |
| GPATCH4 | BC056904.1 | 50.4 | Other | Castello | G-patch | DFCI |
| GPKOW | BC090397.2 | 52.2 | Other | | G-patch | DPCl |
| GRB2 | BC000631.2 | 25.2 | Other | Castello | Other | DFCI |
| GRN | BC000324.2 | 47.0 | Other | Castello | Other | DFCI |
| GRN | BC010577.2 | 63.5 | Other | Castello | Other | DFCI |
| GSPT1 | BC009503.2 | 68.4 | Stability | Baltz/Castello | Other | DFCI |
| GSPT2 | BC036077.1 | 69.0 | Stability | Baltz/Castello | Other | DFCI |
| GTF2E2 | BC030572.2 | 33.0 | Other | Castello | Other | DFCI |
| GTF2F1 | BC000120.1 | 58.2 | Splicing | Baltz/Castello | Other | DFCI |
| GTPBP10 | BC021573.1 | 39.7 | Other | Castello | Other | DFCI |
| GTPSP4 | BC038975.2 | 74.0 | Other | Baltz/Castello | Other | DFCI |
| GTSF1 | BC021179.1 | 19.2 | Other | | Other | DFCI |
| GTSF1L | BC040049.1 | 16.9 | Other | | Other | DFCI |
| HADHB | BC014572.1 | 51.4 | Other | | Other | DFCI |
| HADHB | BC017554.2, | 51.4 | Other | | Other | DFCI |
| | BC030824.1, | | | | | |
| | BC066963.1 | | | | | |
| HDGF | BC018991.1 | 26.8 | Other | Castello | Other | DFCI |
| HEATR1 | BC062442.1 | 13.5 | Other | Baltz/Castello | Other | DFCI |
| HEATR1 | BC011983.1 | 39.9 | Other | Baltz/Castello | Other | DFCI |
| HELQ | BC011863.2 | 30.5 | Other | | Other | DFCI |
| HERC5 | BC140716.1 | 116.8 | Other | Castello | Other | DFCI |
| HFM1 | BC132823.1 | 53.6 | Other | | Other | DFCI |
| HIST1H1C | BC002649.1 | 21.4 | Other | Baltz/Castello | Other | DFCI |
| HIST1H4H | BC120939.2 | 11.4 | Other | Castello | Other | DFCI |
| HMGB1 | BC003378.1 | 24.9 | Other | Baltz/Castello | Other | DFCI |
| HMGB2 | BC001063.2 | 24.0 | Other | Baltz/Castello | Other | DFCI |
| HNRNPA0 | hnRNPA0 | 30.8 | Other | | RRM | Promega |
| HNRNPA1 | BC002335.2, | 34.2 | Localization | Baltz/Castello | RRM | DFCI |
| | BC009800.1, | | | | | |
| | BC812158.1, | | | | | |
| | BC033714.1 | | | | | |
| HNRNPA1 | hnRNPA1 | 34.2 | Other | | RRM | Promega |
| HNRNPA2B1 | BC000506.2 | 28.4 | Localization | Baltz/Castello | RRM | DFCI |
| HNRNPC | BC008423.1 | 33.6 | Splicing | Baltz/Castello | RRM | DFCI |
| HNRNPC | BC003394 | 32.3 | Splicing | Baltz/Castello | RRM | In-house |
| HNRNPC | BC008364 | 32.4 | Splicing | Baltz/Castello | RRM | In-house |
| HNRNPC1/2 | hnRNPC1/2 | 33.7 | Other | | Other | Promega |
| HNRNPCL1 | BC137258.1 | 32.1 | Other | Baltz | RRM | DFCI |
| HNRNPD | BC002401.1 | 38.4 | Stability | Baltz/Castello | RRM | DFCI |
| HNRNPD0 | hnRNPD0 | 32.8 | Other | | Other | Promega |
| HNRNPE1 | hnRNPE1 | 37.5 | Other | | Other | Promega |
| HNRNPE2 | hnRNPE2 | 38.2 | Other | | Other | Promega |
| HNRNPF | BC004254.1 | 45.7 | Splicing | Baltz/Castello | RRM | DFCI |
| HNRNPF | BC001432 | 45.7 | Splicing | Baltz/Castello | RRM | |
| HNRNPF | BC016736 | 45.7 | Splicing | Baltz/Castello | RRM | In-house |
| HNRNPF | hnRNPF | 45.7 | Other | | RRM | Promega |
| HNRNPH | hnRNPH | 51.2 | Other | | Other | Promega |
| HNRNPH1 | BC001348.2 | 49.2 | Splicing | Baltz/Castello | RRM | DFCI |
| HNRNPH2 | BC130345.1 | 49.3 | Splicing | Baltz/Castello | RRM | DFCI |
| HNRNPH2 | | | Splicing | Baltz/Castello | RRM | In-house |
| HNRNPI | hnRMPI | 57.2 | Other | | Other | In-house |
| HNRNPK | BC000355.2 | 51.0 | Splicing | Baltz/Castello | KH | DFCI |
| HNRNPK | BC014980 | 51.0 | Splicing | Baltz/Castello | KH | In-house |
| HNRNPK | hnRNPK | 48.6 | Other | | KH | Promega |
| HNRNPL | hnRNPL | | Other | | RRM | Promega |
| HNRNPM | BC000138 | 77.5 | Splicing | Baltz/Castello | RRM | In-house |
| HNRNPP2 | hnRNPP2 | 53.4 | Other | | Other | Promega |
| HNRNPQ | hnRNPQ | 69.6 | Other | | Other | Promega |
| HNRNPR | BC001449.2 | 71.2 | Splicing | Baltz/Castello | RRM | DFCI |
| HNRNPR | hnRNPR | 70.9 | Other | | RRM | Promega |
| HNRNPU | BC003367 | 89.0 | Stability | Baltz/Castello | Other | In-house |
| HNRNPU | hnRNPU | 89.0 | Other | | Other | In-house |
| HNRNPUL1 | BC027713.2 | 90.3 | Splicing | Baltz/Castello | Other | DFCI |
| HNRNPUL1 | hnRNPUL1 | 95.7 | Other | | Other | Promega |
| HNRNPUL2 | NM_001079559.1 | 85.1 | Other | Baltz/Castello | Other | DNASU |
| HNRPDL | BC007392.2 | 33.6 | Other | Castello | Other | DFCI |
| HNRPLL | BC008217.1 | 30.8 | Other | Baltz/Castello | Other | DFCI |
| HNRPLL | BC017480 | 80.1 | Other | Baltz/Castello | Other | In-house |
| HRSP12 | BC010280.1, | 14.5 | Translation | Castello | Other | DFCI |
| | BC012592.1 | | | | | |
| HSP90AA1 | BC121062.2 | 84.7 | Other | Baltz/Castello | Other | DFCI |
| HSP90AB1 | BC004926.1, | 83.3 | Other | Baltz/Castello | Other | DFCI |
| | BC012807.2 | | | | | |
| HSPA8 | BC007276.1 | 64.6 | Other | Baltz/Castello | Other | DFCI |
| HSPA8 | BC018179.1, | 70.9 | Other | Baltz/Castello | Other | DFCI |
| | BC016680.1 | | | | | |
| HSPA9 | BC000478.2 | 73.7 | Other | Baltz/Castello | Other | DFCI |
| HSPB1 | BC073768, | 22.9 | Other | Castello | Other | DNASU |
| | BC000510 | | | | | |
| HSPD1 | BC003030.1 | 61.1 | Other | Castello | Other | DFCI |
| HSPD1 | BC002676.2 | 61.1 | Other | Castello | Other | DFCI |
| HTATSF1 | BC009896.2 | 85.9 | Other | Castello | RRM | DFCI |
| HTATSF1-DPF3 | nolBC009896 | 85.9 | Other | | Other | In-house |
| HYPE | BC001342 | 50.8 | Other | | Other | In-house |
| IFI16 | BC017059.1 | 82.0 | Other | Castello | Other | DFCI |
| IFIH1 | BC111750.1 | 116.7 | Other | | Other | DFCI |
| IFIH1 | BC046206.1 | 25.1 | Other | | Other | DFCI |
| IFIT2 | BC032839.2 | 56.2 | Other | Castello | Other | DFCI |
| IGF2BP1 | NM_006546.3 | 63.5 | Stability | Baltz/Castello | RRM | DNASU |
| IGF2BP2 | BC021290.2 | 66.0 | Translation | Baltz/Castello | RRM | DFCI |
| IGF2BP3 | BC065269.1 | 63.7 | Translation | Baltz/Castello | RRM | DFCI |
| IGF2BP3 | BC051296.1 | 11.3 | Translation | Baltz/Castello | RRM | DFCI |
| IGFBP6 | | 25.3 | Other | | Other | In-house |
| ILF2 | BC000382.2 | 43.1 | Other | Baltz/Castello | Zn-Finger | DFCI |
| ILF3 | BC064838.1 | 76.5 | Other | Baltz/Castello | dsRBD | DFCI |
| ILF3 | BC003086.1 | 17.8 | Other | Baltz/Castello | dsRBD | DFCI |
| INTS6 | BC039829.1 | 100.4 | Other | | Other | DNASU |
| ISY1 | BC004442.1, | 33.0 | Splicing | Castello | Other | DFCI |
| | BC019849.1 | | | | | |
| KHDRBS2 | BC034043.1 | 38.9 | Other | | KH | DFCI |
| KHDRBS3 | BC032606 | 38.8 | Other | Baltz | KH | In-house |
| KIAA0020 | BC016137.2 | 73.8 | Other | Baltz/Castello | Other | DFCI |
| KIAA1324 | BC125208.1 | 102.0 | Other | | Other | DFCI |
| KIAA1967 | BC018269.1 | 40.8 | Other | Baltz/Castello | Other | DFCI |
| KIF1C | BC034993.1 | 122.9 | Other | Baltz/Castello | Other | DFCI |
| KIF1C | BC111736.1 | 121.9 | Other | Baltz/Castello | Other | DNASU |
| KLKL3 | BC034035 | 34.1 | Other | | Other | In-house |
| KRR1 | BC026107.2 | 43.8 | Other | Baltz/Castello | KH | DFCI |
| KRR1 | BC033867.2 | 43.8 | Other | Baltz/Castello | KH | DFCI |
| KRR1 | BC016778.1 | 43.8 | Other | Baltz/Castello | KH | DFCI |
| KRT18 | BC000698.2 | 48.0 | Other | Castello | Other | DFCI |
| LARP1 | BC001460.2 | 116.5 | Other | Baltz/Castello | Other | DFCI |
| LARP1 | BC010144 | 5.8 | Other | Baltz/Castello | Other | In-house |
| LARP1 | BC033856 | 32.9 | Other | Baltz/Castello | Other | In-house |
| LARP1B | BC030516.1 | 24.1 | Other | Baltz | Other | DFCI |
| LARP1B | BC062606.1 | 29.0 | Other | Baltz | Other | DFCI |
| LARP4 | BC022377.1 | 21.5 | Other | Baltz/Castello | RRM | DFCI |
| LARP4 | BC083479.1 | 42.0 | Other | Baltz/Castello | RRM | DFCI |
| LARP4B | BC131630.1 | 80.6 | Translation | Baltz/Castello | RRM | DFCI |
| LARP8 | BC006082.1, | 54.7 | Translation | | Other | DFCI |
| | BC009446.1, | | | | | |
| | BC014018.2 | | | | | |
| LARP7 | BC066945.1 | 86.9 | Other | Baltz/Castello | RRM | DFCI |
| LGALS1 | BC001693.1, | 14.7 | Other | Castello | Other | DFCI |
| | BC020675.1 | | | | | |
| LGALS3 | BC053667.1 | 26.2 | Other | Castello | Other | DFCI |
| LIN28A | BC028566.2 | 22.7 | Translation | | zf-CCHC | DFCI |
| LIN28B | BC137526.1 | 27.1 | Other | Baltz | zf-CCHC | DFCI |
| LLPH | BC006002.1 | 15.2 | Other | Baltz/Castello | Other | DFCI |
| LSM1 | BC001767.1 | 15.2 | Stability | Baltz/Castello | LSM | DFCI |
| LSM10 | BC007623.1 | 14.1 | Splicing | | LSM | DFCI |
| LSM11 | BC126449 | 39.5 | Other | | LSM | In-house |
| LSM2 | BC009192.2 | 10.8 | Stability | Baltz/Castello | LSM | DFCI |
| LSM3 | BC007055.1 | 11.8 | Stabihty | Baltz/Castello | LSM | DFCI |
| LSM4 | BC000387.2, | 15.3 | Stability | Baltz/Castello | LSM | DFCI |
| | BC003652.2, | | | | | |
| | BC022198.2, | | | | | |
| | BC023665.2 | | | | | |
| LSM5 | BC005938.1 | 9.9 | Stability | | LSM | DFCI |
| LSM6 | BC018026.1 | 9.1 | Stability | Baltz | LSM | DFCI |
| LSM7 | BC018621.1 | 11.8 | Stability | | LSM | DFCI |
| LSMD1 | BC033861.1 | 18.7 | Other | | LSM | DFCI |
| LSMD1 | BC059944.1 | 13.5 | Other | | LSM | DFCI |
| LUC7L | HQ448098 | 41.9 | Other | | Other | DNASU |
| LUC7L2 | BC017163.2, | 46.5 | Other | Baltz/Castello | Other | DFCI |
| | BC050708.2, | | | | | |
| | BC056886.1 | | | | | |
| LUC7L3 | BC056409.1 | 9.2 | Splicing | Baltz/Castello | Other | DFCI |
| LYAR | BC015796.2 | 43.6 | Other | Baltz/Castello | Other | DFCI |
| MAGOHB | BC010905 | 17.3 | Localization | | Other | In-house |
| MAK16 | BC050528.1 | 35.4 | Other | Castello | Ribosomal | DFCI |
| MAP4 | BC008715.2, | 102.9 | Other | Baltz/Castello | Other | DFCI |
| | BC012794.2 | | | | | |
| MATR3 | BC015031 | 94.6 | Other | Baltz/Castello | RRM | DNASU |
| MA2 | BC041629.1 | 28.6 | Other | Baltz/Castello | Zn-Finger | DFCI |
| MBNL1 | BC043493.1 | 41.0 | Splicing | Baltz/Castello | ZnF-CCCH | DFCI |
| MBNL2 | BC104040.1 | 39.3 | Splicing | Baltz/Castello | ZnF-CCCH | DFCI |
| MDH2 | BC001917.1 | 35.5 | Other | Baltz/Castello | Other | DFCI |
| MECP2 | BC011612.1 | 52.4 | Other | Castello | Other | DFCI |
| MBPCE | BC000556.2, | 25.0 | Modification | Baltz/Castello | Other | DFCI |
| | BC018396.1 | | | | | |
| METYL16 | BC050603.1 | 63.6 | Other | Baltz | Other | DFCI |
| METTL3 | BC052244 | 64.4 | Modification | | Other | DNASU |
| MEX3C | NM_018626 | 89.4 | Other | Baltz/Castello | KH | DNASU |
| MFAP1 | BC023557.2, | 52.0 | Other | Baltz/Castello | Other | DFCI |
| | BC050742.1 | | | | | |
| MKI67IP | BC022990.1 | 34.2 | Other | Baltz/Castello | Other | DFCI |
| MKI67IP | BC024238.2 | 34.3 | Other | Baltz/Castello | Other | DFCI |
| MOV10 | BC002548.1, | 113.7 | Other | Baltz/Castello | Other | DFCI |
| | BC009312.2 | | | | | |
| MPHOSPH6 | BC031017.1 | 19.0 | Other | | Other | DNASU |
| MRM1 | BC072411.1 | 38.6 | Other | Baltz/Castello | Other | DFCI |
| MRPL1 | BC014356.1, | 34.5 | Translation | Baltz/Castello | Ribosomal | DFCI |
| | BC032595.1 | | | | | |
| MRPL11 | BC005002.1 | 20.7 | Translation | Baltz/Castello | Ribosomal | DFCI |
| MRPL13 | BC009190.2, | 20.7 | Translation | Baltz/Castello | Ribosomal | DFCI |
| | BC021744.2 | | | | | |
| MRPL3 | BC003375.2 | 38.6 | Translation | Baltz/Castello | Ribosomal | DFCI |
| MRPL30 | BC022391.1 | 18.5 | Other | | Ribosomal | DFCI |
| MRPL32 | BC013147.1 | 21.4 | Translation | Castello | Ribosomal | DFCI |
| MRPL37 | BC000041.2 | 48.1 | Translation | Castello | Other | DFCI |
| MRPL39 | BC004896.2 | 38.2 | Other | Castello | Other | DFCI |
| MRPL4 | BC009856.2 | 34.9 | Translation | Baltz/Castello | Ribosomal | DFCI |
| MRPL41 | BC040035.1 | 15.4 | Translation | Baltz/Castello | Other | DFCI |
| MRPL42 | BC040240.2 | 18.7 | Translation | Castello | Other | DFCI |
| MRPL43 | BC041165.1 | 23.4 | Translation | Castello | Other | DFCI |
| MRPL45 | BC006235.2 | 28.8 | Translation | Baltz/Castello | Other | DFCI |
| MRPL45 | BC130382.1, | 35.4 | Translation | Baltz/Castello | Other | DFCI |
| | BC130384.1 | | | | | |
| MRPS11 | BC012489.1 | 20.5 | Translation | Baltz/Castello | Ribosomal | DFCI |
| MRPS11 | BC032378.1 | 20.6 | Translation | Baltz/Castello | Ribosomal | DFCI |
| MRPS15 | BC031336.1 | 29.8 | Translation | Castello | Ribosomal | DFCI |
| MRPS23 | BC000242.1 | 21.8 | Translation | Castello | Other | DFCI |
| MRPS24 | BC012167.1 | 19.0 | Translation | Baltz/Castello | Other | DFCI |
| MRPS24 | BC054865.1 | 19.0 | Translation | Baltz/Castello | Other | DFCI |
| MRPS30 | BC007735.2 | 50.4 | Translation | Castello | Other | DFCI |
| MRPS31 | BC022045.1 | 45.3 | Other | Baltz/Castello | Other | DFCI |
| MRPS35 | BC015862.1 | 26.4 | Other | Baltz | Other | DFCI |
| MRPS5 | BC014172.2 | 48.0 | Translation | Baltz/Castello | Ribosomal | DFCI |
| MRPS7 | BC000241.1 | 28.2 | Translation | Baltz/Castello | Ribosomal | DFCI |
| MRTO4 | BC003013.1 | 27.6 | Other | Baltz/Castello | Ribosomal | DFCI |
| MSI2 | BC017560.2 | 17.2 | Other | Baltz/Castello | RRM | DFCI |
| MSI2 | BC001526 | 35.2 | Other | Baltz/Castello | RRM | In-house |
| MTDH | BC045642.1 | 63.3 | Other | Baltz/Castello | Other | DFCI |
| MTPAP | BC061703.1 | 66.2 | Other | Baltz/Castello | Other | DFCI |
| MUSK | GQ129313 | 86.4 | Other | | Other | DNASU |
| MYEF2 | BC014533 | 22.4 | Other | Baltz | RRM | In-house |
| NAA15 | BC104806.1 | 101.3 | Other | Castello | Other | DFCI |
| NANOS1 | BC156179 | 30.2 | Translation | | Zn-Finger | DNASU |
| MANOS2 | BC117484.1, | 15.1 | Translation | | Zn-Finger | DFCI |
| | BC117486.1 | | | | | |
| NANOS3 | BC101209.2 | 20.7 | Translation | | Zn-finger | DFCI |
| NAP1L3 | BC034954 | 57.6 | Other | | Other | DNASU |
| NAT10 | BC035558.1 | 115.7 | Other | Baltz/Castello | tRNA | DFCI |
| NCL | BC002343 | 51.0 | Other | Baltz/Castello | RRM | In-house |
| NDUFV3 | BC033766.1 | 11.9 | Other | Baltz/Castello | Other | DFCI |
| NDUFV3 | BC021217.2 | 51.0 | Other | Baltz/Castello | Other | DFCI |
| NGDN | BC030817.1 | 35.9 | Translation | Baltz/Castello | Other | DFCI |
| NGRM | BC001682.2, | 24.4 | Other | Baltz/Castello | Other | DFCI |
| | BC007222.1, | | | | | |
| | BC009389.2, | | | | | |
| | BC017192.2 | | | | | |
| NHP2 | BC000009.2, | 17.2 | Other | Castello | Ribosomal | DFCI |
| | BC006387.2 | | | | | |
| NHP2L1 | BC019282.2 | 14.2 | Splicing | Baltz/Castello | Ribosomal | DFCI |
| NIP7 | BC015941.1 | 20.5 | Other | Baltz/Castello | Other | DFCI |
| NKRF | BC047878.2 | 77.7 | Other | Baltz/Castello | dsRBD | DFCI |
| NMD3 | BC013317.1 | 57.6 | Other | Castello | Other | DFCI |
| NOA1 | BC004894.2 | 78.5 | Translation | | Other | DFCI |
| NOB1 | BC064630.1 | 46.7 | Other | | Other | DFCI |
| NGC2L | BC003555.1 | 84.9 | Other | Baltz/Castello | Other | DFCI |
| NOL10 | BC005125.2 | 80.3 | Other | Baltz/Castello | Other | DFCI |
| NOL12 | BC002808.1 | 24.7 | Other | Baltz/Castello | Other | DFCI |
| NOL7 | BC023517.2 | 29.4 | Other | Castello | Other | DFCI |
| NOL8 | BC146810.1 | 123.8 | Other | Baltz/Castello | RRM | DFCI |
| NOLC1 | BC006769.2 | 44.2 | Other | Baltz/Castello | Other | DFCI |
| NONO | BC010049.2 | 39.0 | Splicing | Baltz/Castello | RRM | DFCI |
| NONO | BC002364.1, | 54.2 | Splicing | Baltz/Castello | RRM | DFCI |
| | BC003129.1, | | | | | |
| | BC012141.1, | | | | | |
| | BC028299.1, | | | | | |
| | BC069639.1 | | | | | |
| NOP10 | BC008866.2 | 7.7 | Other | | Other | DFCI |
| NOP16 | BC040106.1 | 21.2 | Other | Baltz/Castello | Other | DFCI |
| NOP16 | BC032424.2 | 26.6 | Other | Baltz/Castello | Other | DFCI |
| NOP2 | BC106072.1 | 92.9 | Other | Baltz/Castello | Other | DFCI |
| NOP56 | BC004937.1 | 19.5 | Other | Baltz/Castello | Other | DFCI |
| NOP58 | BC032592.2 | 59.6 | Other | Baltz/Castello | Other | DFCI |
| NOP9 | BC025332.1 | 58.2 | Other | | Other | DFCI |
| NOSIP | BC011249.1 | 33.2 | Other | Castello | Other | DFCI |
| NOSIP | BC009299.2, | 33.2 | Other | Castello | Other | DFCI |
| | BC010077.2 | | | | | |
| NOVA1 | BC075038.2 | 51.7 | Splicing | Baltz | KH | DFCI |
| NPM1 | BC009623.2 | 29.5 | Translation | Baltz/Castello | Other | DFCI |
| NPM1 | BC002398.2, | 32.6 | Translation | Baltz/Castello | Other | DFCI |
| | BC008495.1, | | | | | |
| | BC014349.1, | | | | | |
| | BC016716.1, | | | | | |
| | BC018824.1, | | | | | |
| | BC021668.1, | | | | | |
| | BC050628.1 | | | | | |
| NPM1 | BC012566.1 | 32.6 | Translation | Baltz/Castello | Other | DFCI |
| NPM3 | BC054868.1 | 19.3 | Other | Baltz/Castello | Other | DFCI |
| NR5A1 | BC032501 | 51.6 | Other | | Other | In-house |
| NSA2 | BC005288.1 | 30.1 | Other | Baltz/Castello | Ribosomal | DFCI |
| NSUN2 | BC001041.2 | 63.3 | Modification | Baltz/Castello | Other | DFCI |
| NSUN2 | | 63.3 | Modification | Baltz/Castello | Other | DNASU |
| NSUN5 | BC008084.2 | 50.4 | Other | Baltz/Castello | Other | DFCI |
| NUDT16 | BC031215.1 | 17.8 | Other | | Other | DNASU |
| NUDT16L1 | BC006223.2 | 23.3 | Other | Baltz | Other | DNASU |
| NUFIP2 | BC129990.1 | 76.1 | Other | Baltz/Castello | Other | DFCI |
| NUFIP2 | BC108307.1 | 76.1 | Other | Baltz/Castello | Other | DFCI |
| NUP35 | BC047029.1, | 34.8 | Other | | Other | DFCI |
| | BC061896.1 | | | | | |
| NUSAP1 | BC010838.1 | 24.9 | Other | Castello | Other | DFCI |
| NUSAP1 | BC024772.1 | 49.2 | Other | Castello | Other | DFCI |
| NVL | BC012105.1 | 72.7 | Other | Castello | Other | DFCI |
| NXF1 | BC004904.2, | 70.2 | Localization | Baltz/Castello | Other | DFCI |
| | BC028041.1 | | | | | |
| NXF2 | BC015020.1 | 71.6 | Localization | | Other | DFCI |
| NXF3 | BC031616.1 | 60.1 | Localization | | Other | DFCI |
| NXF5 | BC131708.1 | 42.2 | Localization | | Other | DFCI |
| OASL | BC117406.1, | 59.2 | Other | Castello | Other | DFCI |
| | BC117410.1 | | | | | |
| P4HB | BC010859.1, | 57.1 | Other | Baltz/Castello | Other | DFCI |
| | BC029617.1 | | | | | |
| PA2G4 | BC001951.1, | 43.8 | Translation | Castello | Other | DFCI |
| | BC007561.1, | | | | | |
| | BC069786.1 | | | | | |
| PABPC1 | BC015958 | 70.7 | Stability | Baltz/Castello | RRM | DNASU |
| PABPC1P2 | BC068242.1 | 29.9 | Other | | Other | DNASU |
| PABPC3 | BC027617.2 | 70.0 | Other | | RRM | DFCI |
| PABPC5 | BC063113.1 | 43.3 | Other | | RRM | DFCI |
| PABPN1L | BC148673 | 31.5 | Other | | RRM | DNASU |
| PAN3 | BC128179.1 | 62.0 | Stability | | ZnF-CCCH | DNASU |
| PAPD5 | | 63.3 | Other | | Other | In-house |
| PAPD7 | HQ258305 | 59.9 | Other | | Other | DNASU |
| PAPOLA | BC000927.1 | 32.6 | Splicing | | Other | DFCI |
| PAPOLG | BC111701.1 | 82.8 | Other | | Other | DFCI |
| PARN | BC050029.1 | 73.5 | Modification | Castello | Other | DFCI |
| PARP12 | BC081541.1 | 79.1 | Other | Castello | ZnF-CCCH | DFCI |
| PATL1 | BC085264.1, | 70.6 | Stability | Baltz/Castello | Other | DFCI |
| | BC109038.1 | | | | | |
| PCBP1 | BC039742.1 | 37.5 | Splicing | Baltz/Castello | KH | DFCI |
| PCBP2 | BC001155 | 38.2 | Splicing | Baltz/Castello | KH | In-house |
| PCBP3 | BC012061.1 | 33.3 | Other | Castello | KH | DFCI |
| PCBP4 | BC017098.1 | 37.1 | Other | Castello | KH | DFCI |
| PCSK9 | NM_174938.2 | 74.3 | Other | Castello | Other | DNASU |
| PDIA3 | BC038000.1 | 56.8 | Other | Castello | Other | DFCI |
| PDIA3 | BC014433.1 | 56.8 | Other | Castello | Other | DFCI |
| PDIA4 | BC000425.2 | 72.9 | Other | Castello | Other | DFCI |
| PEG10 | BC060659.2 | 37.0 | Other | Baltz/Castello | Other | DFCI |
| PES1 | BC032489.1 | 68.0 | Other | Baltz/Castello | Other | DFCI |
| PHF5A | BC007321.2 | 12.4 | Splicing | Baltz/Castello | Other | DFCI |
| PHF6 | BC005994.1 | 35.3 | Other | Castello | Other | DNASU |
| PINX1 | BC015479.1 | 37.0 | Other | | G-patch | DFCI |
| PIWIL1 | BC028581.2 | 93.5 | Translation | | PIWI/PAZ | DFCI |
| PIWIL2 | BC025995.1 | 109.8 | Translation | | PIWI/PAZ | DFCI |
| PIWIL4 | BC031080.1 | 96.6 | Translation | | PIWI/PAZ | DFCI |
| PKM | BC007640.1 | 57.9 | Other | | Other | DFCI |
| PLRG1 | BC020786.1 | 56.3 | Splicing | | Other | DNASU |
| PNLDC1 | BC112246.1 | 60.1 | Other | | Other | DFCI |
| PNN | BC062602.1 | 81.6 | Stability | Baltz/Castello | Other | DFCI |
| PNO1 | BC008304.1 | 27.9 | Other | Baltz | KH | DFCI |
| PNRC2 | BC001959.1 | 15.6 | Stability | | Other | DNASU |
| POLDIP3 | BC019643.1 | 24.8 | Translation | Baltz/Castello | RRM | DFCI |
| POLK | BC050718.1 | 64.1 | Other | | Other | DFCI |
| POLR2G | BC112162.1 | 19.3 | Splicing | | S1 | DFCI |
| POLR3E | BC000285.1 | 74.8 | Other | | Other | DFCI |
| PORCN | BC895869 | 51.8 | Other | | Other | DNASU |
| POU5F1 | BC117437.1 | 38.6 | Other | Castello | Other | DFCI |
| PPAN | BC033202.1 | 53.2 | Splicing | Baltz/Castello | Other | DFCI |
| PPAN | BC009833.2 | 53.2 | Splicing | Baltz/Castello | Other | DFCI |
| PPAPDC1A | BC101268.2 | 23.5 | Other | | Other | DNASU |
| PPAPDC1A | BC132787.1 | 30.4 | Other | | Other | DNASU |
| PPAPDC1B | BC033025.1 | 26.2 | Other | | Other | DNASU |
| PPAPDC2 | BC038108.2 | 32.3 | Other | | Other | DNASU |
| PPIA | BC000689.2, | 18.0 | Other | Baltz/Castello | Other | DFCI |
| | BC003026.1, | | | | | |
| | BC005320.1, | | | | | |
| | BC013915.1 | | | | | |
| PPIA | BC005982.1 | 18.0 | Other | Baltz/Castello | Other | DFCI |
| PPIB | BC001125 | 23.7 | Other | Baltz/Castello | Other | DNASU |
| PPIE | BC004898.2, | 33.4 | Splicing | Castello | RRM | DFCI |
| | BC008451.1 | | | | | |
| PPIG | BC111693.1 | 87.1 | Other | Baltz/Castello | Other | DFCI |
| PPIG | BC001555.1 | 40.3 | Other | Baltz/Castello | Other | DFCI |
| PPIL4 | BC016984 | 23.1 | Other | Baltz/Castello | RRM | In-hoase |
| PPIL4 | BC018984.1 | 23.2 | Other | Baltz/Castello | RRM | DFCI |
| PPIL4 | BC020986.1 | 57.2 | Other | Baltz/Castello | RRM | DFCI |
| PPP1CA | BC001888.1, | 37.6 | Other | | Other | DNASU |
| | BC004482.2, | | | | | |
| | BC008010.1 | | | | | |
| PPP1CB | AM392772 | 37.2 | Other | | Other | DNASU |
| PRKRA | BC008470.1 | 34.4 | Other | Baltz | dsRBD | DFCI |
| PRMT1 | | 39.4 | Other | Baltz | Other | DNASU |
| PRPF18 | BC000794 | 39.9 | Splicing | | other | DNASU |
| PRPF19 | BC008719.2 | 55.2 | Splicing | | Other | DNASU |
| PRPF3 | BC000184.2, | 77.5 | Splicing | Baltz | Other | DNASU |
| | BC001954.1 | | | | | |
| PRPF31 | BC117389.1 | 55.5 | Splicing | Baltz/Castello | Other | DFCI |
| PRPF38B | BC053838.1 | 21.1 | Splicing | Castello | Other | DFCI |
| PRPF4 | BC001588.2 | 58.4 | Splicing | | Other | DNASU |
| PRPF4 | BC007424.2 | 58.3 | Splicing | | Other | DNASU |
| PRPF40A | BC027178.1 | 25.5 | Splicing | Baltz | Other | DNASU |
| PRPF4B | BC034969 | 117.0 | Splicing | Baltz | Other | DNASU |
| PRPF6 | BC001666.2 | 108.9 | Localization | Castello | Other | DFCI |
| PRPF8 | | 273.6 | Splicing | Baltz/Castello | RRM | DNASU |
| PRR3 | BC126455.1 | 20.6 | Other | Baltz/Castello | ZnF-CCCH | DFCI |
| PRR3 | BC126457.1 | 20.7 | Other | Baltz/Castello | ZnF-CCCH | DFCI |
| PRRC2B | BC012289.1 | 34.4 | Other | Baltz/Castello | Other | DFCI |
| PSMA3 | BC005265 | 28.4 | Other | | Other | In-house |
| PSMC1 | BC000512.2 | 49.2 | Other | | Other | DFCI |
| PSMC1 | BC016368.1 | 49.2 | Other | | Other | DFCI |
| PSMD4 | BC002365.2 | 40.7 | Other | Castello | Other | DFCI |
| PSPC1 | BC014184.2 | 45.6 | Other | Baltz/Castello | RRM | DFCI |
| PTBP1 | BC004383.1 | 67.2 | Splicing | Baltz/Castello | RPM | DFCI |
| PTBP1 | BC002397 | 59.6 | Splicing | Baltz/Castello | RRM | In-house |
| PTBP2 | BC018582 | 67.6 | Splicing | Baltz/Castello | RRM | In-house |
| PTBP3 | BC044585.1 | 60.4 | Splicing | | RRM | DFCI |
| PTCD1 | BC103495.1 | 78.9 | Other | Baltz/Castello | Other | DFCI |
| PTCD2 | BC018720.1 | 26.7 | Other | Baltz/Castello | Other | DFCI |
| PTCD3 | BC011832.2 | 63.1 | Translation | Baltz/Castello | Other | DFCI |
| PTRF | BC065123.1 | 43.5 | Other | | Other | DFCI |
| PTRH1 | BC047012.1 | 22.9 | Other | Castello | tRNA | DFCI |
| PUF60 | BC009734.1 | 54.0 | Splicing | Baltz/Castello | RRM | DFCI |
| PUF60 | BC011265.1, | 55.7 | Splicing | Baltz/Castello | RRM | DFCI |
| | BC011879.1 | | | | | |
| PUF60 | BC008875.2 | 58.2 | Splicing | Baltz/Castello | RRM | DFCI |
| PUM1 | BC013398.2 | 126.5 | Translation | Baltz/Castello | Pumilio | DFCI |
| PURB | BC101735.1 | 33.2 | Other | Baltz/Castello | Other | DFCI |
| PURG | BC106708.2 | 39.6 | Other | Baltz | Other | DFCI |
| PUS7 | BC005209.2 | 29.8 | Modification | Baltz/Castello | Other | DFCI |
| PUS7 | BC011398 | 29.8 | Modification | | Other | In-house |
| PUS7L | BC068502.1 | 80.7 | Modification | | Other | DFCI |
| PUS7L | BC033621.2 | 80.7 | Modification | | Other | DFCI |
| PWP2 | BC013309.2, | 102.4 | Other | Baltz/Castello | Other | DFCI |
| | BC014986.1 | | | | | |
| OARS | BC001772.1 | 69.8 | Translation | | tRNA | DFCI |
| OARS | BC016634.1 | 69.8 | Translation | | TRNA | DFCI |
| OKI | BC019917.2 | 37.7 | Translation | Baltz | KH | DFCI |
| RALY | BC103753.1 | 32.6 | Splicing | Baltz/Castello | RRM | DFCI |
| RALYL | BC031090.1 | 32.3 | Other | Castello | RRM | DFCI |
| RALYL | HQ447147 | 32.3 | Other | Castello | RRM | DNASU |
| RAN | BC014518.2, | 24.4 | Localization | Baltz/Castello | Other | DFCI |
| | BC014901.2, | | | | | |
| | BC016654.1, | | | | | |
| | BC051908.2 | | | | | |
| RBBP6 | BC029352.1 | 13.2 | Other | Baltz | zf-CCHC | DNASU |
| RBBP6 | BC172357 | 201.6 | Other | Baltz | zf-CCHC | DNASU |
| RBFOX1 | BC113691.1 | 42.4 | Localization | | RRM | DFCI |
| RBFOX2 | BC025281.1 | 40.4 | Splicing | Baltz/Castello | RRM | DFCI |
| RBFQX2 | BC013115.1 | 37.9 | Splicing | Baltz/Castello | RRM | DFCI |
| RBFOX3 | RBFOX3 | 33.9 | Splicing | | RRM | In-house |
| RBM10 | BC004181.2, | 103.5 | Stability | Baltz/Castello | RRM | DFCI |
| | BC008733.2, | | | | | |
| | BC024153.2 | | | | | |
| RBM11 | BC030196.1 | 32.2 | Splicing | | RRM | DFCI |
| RBM12 | BC012787.2, | 97.4 | Other | Castello | RRM | DFCI |
| | BC013981.2 | | | | | |
| RBM12B | BC039260.1 | 87.4 | Other | Baltz/Castello | RRM | DFCI |
| RBM14 | BC000488.2 | 69.5 | Other | Baltz/Castello | RRM | DFCI |
| RBM15 | BC103493.1 | 106.4 | Other | Baltz/Castello | RRM | DFCI |
| RBM15B | BC139836.1 | 63.1 | Splicing | Baltz/Castello | RRM | DFCI |
| RBM17 | BC039322.1 | 46.0 | Splicing | | RRM | DFCI |
| RBM18 | BC008942.2 | 21.8 | Other | | RRM | DFCI |
| RBM19 | BC004289.1, | 107.3 | Other | Baltz/Castello | RRM | DFCI |
| | BC006137.1 | | | | | |
| RBM22 | AL136933 | 46.9 | Splicing | Baltz/Castello | RRM | DNASU |
| RBM23 | BC002586.2 | 46.8 | Other | | RRM | DFCI |
| RBM24 | BC104810.1 | 19.8 | Stability | | RRM | DFCI |
| RBM25 | BC136775.1 | 100.2 | Splicing | Baltz/Castello | RRM | DFCI |
| RBM26 | BC000791.2 | 7.3 | Other | Baltz/Castello | RRM | DFCI |
| RBM26 | BC111697.1 | 111.0 | Other | Baltz/Castello | RRM | DFCI |
| RBM26 | BC041655.1 | 110.7 | Other | Baltz/Castello | RRM | DFCI |
| RBM28 | BC013889.2 | 85.7 | Splicing | Baltz/Castello | RRM | DFCI |
| RBM3 | BC006825.1 | 17.2 | Translation | Baltz/Castello | RRM | DFCI |
| RBM33 | BC011923.2 | 30.2 | Other | Baltz/Castello | RRM | DFCI |
| RBM34 | BC029451.1 | 48.1 | Other | Baltz/Castello | RRM | DFCI |
| RBM38 | BC018711 | 23.4 | Stability | Baltz/Castello | RRM | DNASU |
| RBM4 | BC021120.1 | 20.0 | Splicing | Baltz/Castello | RRM | DFCI |
| RBM4 | BC032735.1 | 40.3 | Splicing | Baltz/Castello | RRM | DFCI |
| RBM41 | BC006986 | 47.1 | Other | | RRM | DNASU |
| RBM42 | BC002868.2 | 47.4 | Other | Castello | RRM | DFCI |
| RBM42 | BC004204.2 | 50.4 | Other | Castello | RRM | DFCI |
| RBM43 | BC136411.1 | 40.7 | Other | | RRM | DNASU |
| RBM45 | BC086549.1 | 53.3 | Other | Baltz/Castello | RRM | DFCI |
| RBM46 | BC028588.2 | 60.0 | Other | | RRM | DFCI |
| RBM47 | BC126261.1 | 64.1 | Other | Baltz/Castello | RRM | DFCI |
| RBM48 | BC003503.1, | 40.1 | Translation | Baltz/Castello | RRM | DNASU |
| | BC004951.1 | | | | | |
| RBM5 | BC002957.1 | 81.5 | Splicing | Baltz | RRM | DFCI |
| RBM6 | BC046643.1 | 69.2 | Other | Baltz/Castello | RRM | DFCI |
| RBM7 | BC034381.1 | 30.5 | Other | Baltz/Castello | RRM | DFCI |
| RSM8A | GQ0120283 | 19.9 | Stability | Baltz | RRM | DNASU |
| RBMS1 | BC085192.1, | 6.7 | Other | Baltz/Castello | RRM | DFCI |
| | BC080620.1 | | | | | |
| RBMS1 | BC018951.2 | 44.5 | Other | Baltz/Castello | RRM | DFCI |
| RBMS2 | BC027863.1 | 44.0 | Other | Baltz/Castello | RRM | DFCI |
| RBMX | BC006550.2 | 42.3 | Splicing | Baltz/Castello | RRM | DFCI |
| RBMX2 | BC033750.1 | 37.3 | Other | Castello | RRM | DFCI |
| RBMX2 | BC125126.1 | 74.0 | Other | Castello | RRM | DNASU |
| RBMY1A1 | X76059 | 40.7 | Splicing | | RRM | DNASU |
| RBMY1A1 | BC070298.1 | 51.3 | Splicing | | RRM | DNASU |
| RBMY1F | BC030018.2 | 55.7 | Splicing | | RRM | DFCI |
| RBPMS | BC003608.2 | 24.3 | Other | Caatello | RRM | DFCI |
| RC3H2 | BC044642.1 | 56.9 | Other | Baltz/Castello | RRM | DFCI |
| RDBP | BC025235.1, | 43.2 | Other | Castello | Other | DFCI |
| | BC050617.2 | | | | | |
| RDX | BC047109.1 | 68.6 | Ottrer | Castello | Other | DFCI |
| REPIN1 | BC001760.1 | 63.6 | Other | Castello | Zn-Finger | DFCI |
| REXO4 | BC009274.2 | 46.7 | Other | Baltz/Castello | Other | DFCI |
| REXO5 | BC007646 | 86.9 | Other | | Other | In-house |
| RNMTL1 | BC050614.1 | 47.0 | Other | Baltz/Castello | Other | DFCI |
| RNMTL1 | BC011550.1 | 47.0 | Other | Baltz/Castello | Other | DFCI |
| RNPC3 | BC010697.1 | 30.1 | Splicing | | RRM | DFCI |
| RNPS1 | BC001659.2 | 34.2 | Stability | Baltz/Castello | RRM | DFCI |
| RPGR | BC031624.1 | 52.6 | Other | Castello | Other | DFCI |
| RPL10A | BC006791.1, | 24.8 | Stability | Baltz/Castello | Ribosomal | DFCI |
| | BC011366.1 | | | | | |
| RPL13A | BC000514.2, | 23.6 | Stability | Castello | Ribosomal | DFCI |
| | BC001675.2, | | | | | |
| | BC065236.1 | | | | | |
| RPL13A | BC070223.1 | 23.6 | Stability | Castello | Ribosomal | DFCI |
| RPL14 | BC005134.2 | 23.9 | Stability | Baltz/Castello | Ribosomal | DFCI |
| RPL14 | BC009294.2 | 23.6 | Stability | Baltz/Castello | Ribosomal | DFCI |
| RPL15 | BC014837.1 | 24.1 | Stability | Baltz/Castello | Ribosomal | DFCI |
| RPL15 | BC071672.1 | 24.1 | Stability | Baltz/Castello | Ribosomal | DFCI |
| RPL15 | BC081585.1 | 16.7 | Stability | Baltz/Castello | Ribosomal | DFCI |
| RPL18A | BC066319.1 | 20.8 | Stability | Baltz/Castello | Ribosomal | DFCI |
| RPL19 | BC000530.2, | 23.5 | Stability | Castello | Ribosomal | DFCI |
| | BC013016.2 | | | | | |
| RPL21 | BC091603.1, | 18.6 | Stability | Baltz/Castello | Ribosomal | DFCI |
| | BC007505.2, | | | | | |
| | BC071902.1 | | | | | |
| RPL22 | BC058887.1 | 14.8 | Stability | Baltz/Castello | Ribosomal | DFCI |
| RPL23 | BC010114.2 | 14.9 | Stability | Castello | Ribosomal | DFCI |
| RPL23 | BC062716.1 | 14.9 | Stability | Castello | Ribosomal | DFCI |
| RPL23A | BC014459.1 | 17.7 | Stability | Baltz/Castello | Ribosomal | DFCI |
| RPL27 | BC001700.2, | 15.8 | Stability | Baltz/Castello | Ribosomal | DFCI |
| | BC002588.2, | | | | | |
| | BC007273.1, | | | | | |
| | BC010026.2 | | | | | |
| RPL28 | BC010173.2, | 15.7 | Stability | Castello | Ribosomal | DFCI |
| | BC011582.1 | | | | | |
| RPL23 | BC010182.2 | 15.8 | Stability | Castello | Ribosomal | DFCI |
| RPL3 | BC004323.1 | 26.8 | Stability | Baltz/Castello | Ribosomal | DFCI |
| RPL3 | BC006483.1, | 48.1 | Stability | Baltz/Castello | Ribosomal | DFCI |
| | BC008003.1, | | | | | |
| | BC012786.2, | | | | | |
| | BC014017.2, | | | | | |
| | BC063662.1 | | | | | |
| RPL30 | BC032700.2 | 12.8 | Stability | Baltz/Castello | Ribosomal | DFCI |
| RPL31 | BC017343.2 | 14.5 | Stability | Castello | Ribosomal | DFCI |
| RPL32 | BC011514.1 | 15.9 | Stability | Castello | Ribosomal | DFCI |
| RPL35 | BC010918.1 | 14.6 | Stability | Castello | Ribosomal | DFCI |
| RPL35 | BC000348.2 | 14.6 | Stability | Castello | Ribosomal | DFCI |
| RPL35A | BC001037.2, | 12.5 | Stability | Baltz/Castello | Ribosomal | DFCI |
| | BC010949.1, | | | | | |
| | BC017093.1, | | | | | |
| | BC081890.1 | | | | | |
| RPL36 | BC004971.1 | 12.3 | Stability | Castello | Ribosomal | DFCI |
| RPU | BC001365.2, | 47.7 | Stability | Baltz/Castello | Ribosomal | DFCI |
| | BC005817.2, | | | | | |
| | BC007748.2, | | | | | |
| | BC007996.1, | | | | | |
| | BC009888.2, | | | | | |
| | BC010151.2, | | | | | |
| | BC014653.1, | | | | | |
| | BC068925.1 | | | | | |
| RPL5 | BC001882.1 | 12.1 | Stability | Baltz/Castello | Ribosomal | DFCI |
| RPL6 | BC004138.2, | 32.7 | Stability | Castello | Ribosomal | DFCI |
| | BC032299.1 | | | | | |
| RPL6 | BC022444.1 | 32.7 | Stability | Castello | Ribosomal | DFCI |
| RPL7 | BC006095.1, | 29.2 | Stability | Baltz/Castello | Ribosomal | DFCI |
| | BC008850.2, | | | | | |
| | BC009599.1 | | | | | |
| RPL7L1 | BC073890.1 | 28.7 | Other | Baltz/Castello | Ribosomal | DFCI |
| RPL8 | BC00077.2 | 28.0 | Stability | Baltz/Castello | Ribosomal | DFCI |
| RPL8 | BC012197.1, | 28.0 | Stability | Baltz/Castello | Ribosomal | DFCI |
| | BC013104.1 | | | | | |
| RPLP0 | BC000087.2, | 34.3 | Stability | Baltz/Castello | Ribosomal | DFCI |
| | BC008092.1, | | | | | |
| | BC008594.1, | | | | | |
| | BC009867.2, | | | | | |
| | BC015173.1, | | | | | |
| | BC015690.1 | | | | | |
| RPLP0 | BC000345.2, | 34.3 | Stability | Baltz/Castello | Ribosomal | DFCI |
| | BC000752.2, | | | | | |
| | BC003655.2 | | | | | |
| RPN1 | BC010839.1 | 68.6 | Other | Castello | Other | DFCI |
| RPP21 | BC011730.2 | 17.6 | Other | | Other | DFCI |
| RPP25 | BC002497.2, | 20.6 | Other | Castello | Other | DFCI |
| | BC007270.1 | | | | | |
| RPP30 | BC006991.1 | 29.3 | Other | Castello | Other | DFCI |
| RPS10 | BC001032.2, | 18.9 | Stability | Baltz/Castello | Other | DFCI |
| | BC001955.1, | | | | | |
| | BC005012.1 | | | | | |
| RPS11 | BC007283.1, | | | | | |
| | BC007603.1, | | | | | |
| | BC007945.2, | | | | | |
| | BC010028.2, | | | | | |
| | BC016378.1 | | | | | |
| RPS15A | BC001697.2 | 14.8 | Stability | Baltz/Castello | Ribosomal | DFCI |
| RPS15A | BC030569.1, | 14.8 | Stability | Baltz/Castello | Ribosomal | DFCI |
| | BC048113.1 | | | | | |
| RPS19BP1 | BC037573.1, | 15.4 | Other | Baltz/Castello | Other | DFCI |
| | BC047711.1 | | | | | |
| RPS2 | BC018178.1 | 31.3 | Stability | Baltz/Castello | Ribosomal | DFCI |
| RPS2 | BC001795.1, | 31.3 | Stability | Baltz/Castello | Ribosomal | DFCI |
| | BC008862.2, | | | | | |
| | BC010165.2, | | | | | |
| | BC016951.2, | | | | | |
| | BC021545.1, | | | | | |
| | BC023354.1 | | | | | |
| RPS20 | BC007507.2 | 13.4 | Stability | Baltz/Castello | Ribosomal | DFCI |
| RPS21 | BC027976.1 | 7.1 | Stability | Castello | Ribosomal | DFCI |
| RPS24 | BC000523.2 | 15.1 | Stability | Baltz/Castello | Ribosomal | DFCI |
| RPS28 | BC000354.1, | 7.8 | Stability | Baltz/Castello | Ribosomal | DFCI |
| | BC021239.2 | | | | | |
| RPS3 | BC003137.1, | 26.7 | Stability | Baltz/Castello | KH | DFCI |
| | BC034149.1 | | | | | |
| RPS3A | BC000204.1, | 29.9 | Stability | Baltz/Castello | Ribosomal | DFCI |
| | BC001708.1, | | | | | |
| | BC004981.1, | | | | | |
| | BC006298.2, | | | | | |
| | BC009219.2, | | | | | |
| | BC009404.2, | | | | | |
| | BC017123.2, | | | | | |
| | BC019072.2, | | | | | |
| | BC030161.2 | | | | | |
| RPS4X | BC007308.1 | 22.2 | Stability | Baltz/Castello | Ribosomal | DFCI |
| RPS4X | BC100903.1 | 29.6 | Stability | Baltz/Castello | Ribosomal | DFCI |
| RPS6 | BC013296.2 | 28.7 | Stability | Castello | Ribosomal | DFCI |
| RPS6 | BC027620.1 | 28.7 | Stability | Castello | Ribosomal | DFCI |
| RPS7 | BC002886.2, | 22.1 | Stability | Baltz/Castello | Ribosomal | DFCI |
| | BC061901.1 | | | | | |
| RPS8 | BC070875.1 | 24.2 | Stability | Baltz/Castello | Ribosomal | DFCI |
| RPUSD3 | BC065741.1 | 37.6 | Modification | Baltz/Castello | Other | DFCI |
| RPUSD3 | BC032135.2 | 37.5 | Modification | Baltz/Castello | Other | DFCI |
| RPUSD4 | BC014131.2 | 42.2 | Modification | Baltz/Castello | Other | DFCI |
| RRP36 | BC011933.2 | 29.8 | Other | Baltz/Castello | Other | DFCI |
| RRP7A | BC073834.1, | 32.3 | Other | Castello | RRM | DFCI |
| | BC121118.1 | | | | | |
| RRP8 | BC001071.2 | 50.7 | Other | Baltz/Castello | Other | DFCI |
| RRS1 | BC001811.2, | 41.2 | Other | Baltz/Castello | Other | DFCI |
| | BC013043.2 | | | | | |
| RSL1D1 | BC113899.1 | 55.0 | Other | Baltz/Castello | Ribosomal | DFCI |
| RSRC1 | HQ448170 | 38.7 | Splicing | | Other | DNASU |
| RTCA | BC012804.1 | 40.7 | Other | | Other | DFCI |
| RTN4 | BC016165.1 | 42.3 | Other | Castello | Other | DFCI |
| RTN4 | BC012619.1 | 40.3 | Other | Castello | Other | DFCI |
| RY1 | BC017890.1 | 18.9 | Other | | Other | DNASU |
| S100A4 | BC018300.1 | 11.7 | Other | Castello | Other | DFCI |
| SAFB2 | BC025279.1 | 57.2 | Other | Baltz/Castello | RRM | DFCI |
| SAMD4A | BC057836.1 | 27.6 | Translation | Castello | SAM | DFCI |
| SAMD4A | BC121173.1 | 70.0 | Translation | Castello | SAM | DFCI |
| SAMSN1 | BC029112.1 | 41.7 | Other | Castsllo | Other | DFCI |
| SARNP | BC007099.1 | 23.7 | Translation | Baltz/Castello | Other | DFCI |
| SART3 | BC111883.1 | 109.9 | Other | Baltz/Castello | RRM | DFCI |
| SBDS | BC065700.1 | 28.8 | Other | Castello | Other | DFCI |
| SCAF8 | BC070071.1 | 140.5 | Splicing | | RRM | DFCI |
| SCD5 | BC004936.1 | 14.3 | Other | | Other | DFCI |
| SCD5 | | 14.3 | Other | | Other | DNASU |
| SCG3 | BC014539.1 | 53.0 | Other | Castello | Other | DFCI |
| SEC61B | BC001734.1 | 10.0 | Other | Cassette | Other | DFCI |
| SEC63 | BC048287.1 | 88.0 | Other | Castello | Other | DFCI |
| SEC63 | BC047221.1 | 88.0 | Other | Castello | Other | DFCI |
| SECISBP2 | BC001189.1 | 95.5 | Translation | Castello | Ribosomal | DFCI |
| SECP43 | | 32.5 | Other | | Other | In-house |
| SENP5 | BC030705 | 86.7 | Other | | Other | In-house |
| SERBP1 | BC020555.1 | 44.3 | Stability | Baltz/Castello | Other | DFCI |
| SERBP1 | BC002488.2 | 44.2 | Stability | Baltz/Castello | Other | DFCI |
| SERBP1 | BC003049.1, | 45.0 | Stability | Baltz/Castello | Other | DFCI |
| | BC008045.2, | | | | | |
| | BC019273.1, | | | | | |
| | BC026918.1 | | | | | |
| SERPINH1 | BC014623 | 46.4 | Other | Castello | Other | DNASU |
| SF1 | BC008080.2, | 59.7 | Splicing | Baltz/Castello | KH | DFCI |
| | BC020217.1 | | | | | |
| SF3A1 | BC001976.1, | 88.9 | Splicing | Baltz/Castello | Other | DFCI |
| | BC097684.2 | | | | | |
| SF3A2 | BC004434 | 49.3 | Splicing | Baltz/Castello | Zn-finger | DNASU |
| SF3A3 | BC011523 | 58.8 | Splicing | Baltz/Castello | Other | DNASU |
| SF3B14 | BC015413 | 13.6 | Splicing | Baltz | RRM | In-house |
| SF3B14 | BC016483.1 | 14.6 | Splicing | Baltz | RRM | DNASU |
| SF3B2 | BC007610.1 | 72.6 | Splicing | Baltz/Castello | Other | DFCI |
| SF3B3 | JF432652 | 30.2 | Splicing | | Other | DNASU |
| SF3B3 | BC009463.1 | 44.8 | Splicing | | Other | DNASU |
| SF3B4 | BC004273.1, | 44.4 | Splicing | Baltz/Castello | RRM | DFCI |
| | BC013886.2 | | | | | |
| SFPQ | BC051192.1 | 76.2 | Splicing | Baltz/Castello | RRM | DFCI |
| SFRS10 | BC000160 | 33.7 | Other | | Other | DNASU |
| SFRS13A | HQ448699 | 22.2 | Other | | Other | DNASU |
| SFRS16 | BC013178.1 | 23.3 | Other | | Other | DNASU |
| SFRS17A | BC028151.1 | 51.5 | Other | | Other | DNASU |
| SFRS17A | BC110496.1 | 80.7 | Other | | Other | DNASU |
| SFRS2IP | NM_004719 | 164.7 | Other | | Other | DNASU |
| SFSWAP | BC136678.1 | 104.8 | Splicing | | Other | DFCI |
| SKIV2L | BC015758 | 137.8 | Other | | DEAD | DNASU |
| SKIV2L2 | BC028604.2 | 117.8 | Splicing | Castello | DEAD | DFCI |
| SLBP | BC014908.1, | 31.3 | Localization | Castello | Other | DFCI |
| | BC015703.1 | | | | | |
| SLC25A5 | BC058160.1 | 32.9 | Other | Castello | Other | DFCI |
| SLC3A2 | BC001061.2, | 57.9 | Other | Castello | Other | DFCI |
| | BC003000.1 | | | | | |
| SLC7A9 | BC017962.1 | 53.5 | Other | | Other | DFCI |
| SLIRP | BC017895.1 | 12.3 | Other | Baltz | RRM | DFCI |
| SLU7 | BC010634.1 | 68.3 | Splicing | | Other | DNASU |
| SMG6 | BC148373 | 57.3 | Stability | | Other | DNASU |
| SMN1 | BC015308 | 31.8 | Other | | Other | DNASU |
| SMN1 | BC000908 | 30.4 | Other | | Other | DNASU |
| SMNDC1 | BC011234.1 | 26.7 | Splicing | Castello | Other | DFCI |
| SNIPI | BC027040.1 | 45.8 | Other | Castello | Other | DFCI |
| SNRNP35 | BC009622.1 | 30.0 | Splicing | | RRM | DFCI |
| SNRNP40 | BC001494.2 | 39.3 | Splicing | Castello | Other | DFCI |
| SNRNP70 | BC001315.1 | 51.6 | Splicing | Baltz/Castello | RRM | DFCI |
| SNRPA | BC000405.2, | 31.3 | Splicing | Baltz | RRM | DFCI |
| | BC008290.1 | | | | | |
| SNRPB | BC080516.1 | 23.7 | Splicing | Baltz | LSM | DFCI |
| SNRPB2 | BC018022.1, | 25.5 | Splicing | | RRM | DFCI |
| | BC038737.2 | | | | | |
| SNRPD2 | BC000488.2 | 13.5 | Splicing | Castello | LSM | DFCI |
| SNRPE | BC002639.2 | 10.8 | Splicing | | LSM | DFCI |
| SNRPF | BC063397.1, | 9.7 | Splicing | | LSM | DFCI |
| | BC128453.1 | | | | | |
| SNRPG | BC000070.2, | 8.5 | Splicing | Castello | LSM | DFCI |
| | BC022432.1, | | | | | |
| | BC086302.1 | | | | | |
| SNRPN | BC003180.1, | 24.6 | Splicing | | LSM | DFCI |
| | BC010057.1, | | | | | |
| | BC024777.1, | | | | | |
| | BC025178.1 | | | | | |
| SNURF | BC024777.1 | 24.6 | Other | | Other | DNASU |
| SNW1 | BC108903.1 | 81.6 | Splicing | Baltz | Other | DNASU |
| SOX21 | BC111584 | 28.6 | Other | | Other | DNASU |
| SPATS2 | BC048299.1 | 59.5 | Other | Castello | Other | DFCI |
| SPATS2L | BC018738.1 | 64.0 | Other | Baltz/Castello | Other | DFCI |
| SR140 | BC111692.1 | 72.5 | Other | | Other | DNASU |
| SRBD1 | BC032538.1 | 69.7 | Other | | S1 | DFCI |
| SREK1 | BC067770.1, | 69.4 | Splicing | Baltz | RRM | DFCI |
| | BC112343.1 | | | | | |
| SRFBP1 | BC031222.1 | 48.6 | Other | Baltz/Castello | Other | DFCI |
| SRP14 | BC030495.2 | 14.6 | Translation | Baltz/Castello | Other | DFCI |
| SRP68 | BC020238.1 | 67.3 | Translation | Castello | Other | DFCI |
| SRPK2 | BC035214.1 | 77.6 | Splicing | Baltz/Castello | Other | DFCI |
| SRPK2 | BC068547.1 | 77.6 | Splicing | Baltz/Castello | Other | DFCI |
| SRPR | BC001162.1, | 69.8 | Translation | Castello | Other | DFCI |
| | BC009110.1, | | | | | |
| | BC013583.1 | | | | | |
| SRPR | BC008077.2 | 63.2 | Translation | Castello | Other | DFCI |
| SRSF1 | BC010264.1 | 27.7 | Localization | Baltz/Castello | RRM | DFCI |
| SRSF10 | BC001107.1 | 22.2 | Localization | Baltz | RRM | DFCI |
| SRSF10 | BC005039.1 | 31.3 | Localization | Baltz | RRM | DFCI |
| SRSF11 | BC040436.1 | 53.4 | Localization | Castello | RRM | DFCI |
| SRSF12 | BC021715.1 | 30.5 | Splicing | Baltz | RRM | DFCI |
| SRSF2 | BC066958.1 | 19.4 | Localization | Castello | RRM | DFCI |
| SRSF3 | BC000914.1, | 19.3 | Localization | Baltz/Castello | RRM | DFCI |
| | BC069018.1 | | | | | |
| SRSF4 | BC002781.2 | 56.7 | Localization | Castello | RRM | DFCI |
| SRSF5 | BC018823.2 | 31.3 | Localization | Castello | RRM | DFCI |
| SRSF6 | BC006832.2 | 39.6 | Localization | Castello | RRM | DFCI |
| SRSF7 | BC000997.2, | 27.4 | Localization | Castello | RRM | DFCI |
| | BC017369.2, | | | | | |
| | BC022328.1 | | | | | |
| SRSF8 | BC057783.1 | 31.4 | Other | Bahz/Castello | RRM | DFCI |
| SRSF9 | BC093971.1 | 25.5 | Localization | Castello | RRM | DFCI |
| SSBP1 | BC000895.1 | 17.3 | Other | Baltz/Castello | Other | DFCI |
| SSRP1 | BC005116.1 | 81.1 | Other | Baltz/Castello | Other | DFCI |
| STAU1 | BC050432.1 | 63.2 | Localization | Baltz | dsRBD | DFCI |
| STAU2 | BC008370.1 | 11.8 | Other | Baltz/Castello | dsRBD | DFCI |
| STAU2 | BC110447.1 | 52.8 | Other | Baltz/Castello | dsRSD | DFCI |
| STAU2 | BC008369.1 | 59.0 | Other | Baltz/Castello | dsRBD | DFCI |
| STIP1 | BC002987.1 | 62.6 | Other | Castello | Other | DFCI |
| STRBP | BC017732.1 | 73.7 | Other | Baltz/Castello | dsRBD | DFCI |
| STXBP1 | BC015749.1 | 67.6 | Other | Castello | Other | DFCI |
| SUCLG1 | BC000504.2 | 35.0 | Other | Castello | Other | DFCI |
| SUGP1 | BC142988.1 | 72.5 | Splicing | | G-patch | DFCI |
| SUGP2 | BC020586.1 | 110.4 | Splicing | Baltz/Castello | G-patch | DFCI |
| SUMO1 | BC006462.1, | 11.8 | Other | Castello | Other | DFCI |
| | BC053528.1 | | | | | |
| SUMO1 | BC066308.1 | 11.5 | Other | Castello | Other | DFCI |
| SUPT5H | BC024203.2 | 121.0 | Other | Baltz/Castello | Other | DFCI |
| SUPV3L1 | BC036112.1 | 88.0 | Other | Baltz/Castello | Other | DFCI |
| SURF6 | BC014878.1 | 41.5 | Other | Baltz/Castello | Other | DFCI |
| SYF2 | BC010882.1 | 28.7 | Splicing | Baltz/Castello | Other | DFCI |
| SYMPK | BC006567 | 126.6 | Other | | Other | DNASU |
| SYNCRIP | BC032843.1 | 58.7 | Stability | Baltz/Castello | RRM | DFCI |
| TAF15 | BC046099.1 | 81.8 | Other | Baltz/Castello | RRM | DFCI |
| TAF15 | TAF15(1) | 61.8 | Other | | Other | In-house |
| TAf15 | TAF15(5) | 61.3 | Other | | Other | In-house |
| TARDBP | BC001487 | 29.5 | Stability | Baltz/Castello | RRM | In-house |
| TARDBP | BC095435 | 44.9 | Stability | Baltz/Castello | RRM | DNASU |
| TBRG4 | BC014918.1, | 70.7 | Other | Baltz/Castello | Other | DFCI |
| | BC017235.2 | | | | | |
| TCOF1 | | 96.8 | Other | Baltz/Castello | Other | DNASU |
| TDRD3 | BC030514.1 | 73.2 | Other | Baltz/Castello | Other | DFCI |
| TDRD9 | BC128057.1 | 99.5 | Other | | DEAD | DFCI |
| TDRKH | BC032890.1 | 82.1 | Other | | KH | DFCI |
| TEFM | BC024328.1 | 26.3 | Other | Baltz | Other | DFCI |
| TERT | BC172541 | 125.7 | Other | | Other | DNASU |
| TES | BC001451.1 | 48.0 | Other | Castello | Other | DFCI |
| TFB1M | BC017788.1 | 39.5 | Modification | Castello | Other | DFCI |
| TFIP11 | BC011599.2 | 96.8 | Splicing | | G-patch | DFCI |
| THOC1 | BC010381 | 75.7 | Localization | | Other | DNASU |
| THOC3 | BC006849 | 38.8 | Localization | | Other | DNASU |
| THOC4 | BC052302.1 | 27.6 | Other | Baltz/Castello | Other | DNASU |
| THOC5 | CU013430 | 78.5 | Localization | | Other | DNASU |
| THOC6 | | 37.5 | Localization | | Other | DNASU |
| THRAP3 | BC112330.1 | 108.7 | Stability | Baltz/Castello | Other | DFCI |
| THUMPD1 | BC000448.2 | 39.3 | Other | Castello | Other | DFCI |
| TIA1 | BC015944 | 24.1 | Splicing | Baltz/Castello | RRM | In-house |
| TIAL1 | BC030025.1 | 27.9 | Other | Baltz/Castello | RRM | DFCI |
| TOB1 | DQ893993 | 38.1 | Other | | Other | DNASU |
| TOB2 | BC038957 | 36.6 | Other | | Other | DNASU |
| TOE1 | JF432161 | 56.5 | Other | | Other | DNASU |
| TOE1 | | 56.5 | Other | | Other | In-house |
| TOP3B | BC002432.2 | 96.7 | Other | Baltz/Castello | Other | DFCI |
| TPD52L2 | BC008804.1 | 22.2 | Other | Castello | Other | DFCI |
| TFT1 | BC022436.1 | 15.6 | Other | Castello | Other | DFCI |
| TPT1 | BC003352.2, | 19.6 | Other | Castello | Other | DFCI |
| | BC052333.1 | | | | | |
| TRA2A | BC017094.2 | 32.7 | Splicing | Baltz/Castello | RRM | DFCI |
| TRAP1 | BC018950.2 | 80.1 | Other | Castello | Other | DFCI |
| TRIM39 | BC034985.1 | 56.4 | Other | | Other | DFCI |
| TRIM39 | BC097661.2 | 56.4 | Other | | Other | DFCI |
| TRIM56 | BC048194.1 | 81.5 | Other | Baltz/Castello | Zn-Finger | DFCI |
| TRIP6 | BC004999.1, | 50.3 | Other | Castello | Other | DFCI |
| | BC028985.1 | | | | | |
| TRIP6 | BC002680.2 | 50.1 | Other | Castello | Other | DFCI |
| TRMT10C | BC035967.1 | 46.7 | Other | | tRNA | DFCI |
| TRMT1L | BC045535.1 | 81.7 | Other | Castello | Other | DFCI |
| TRMT2A | BC013352.2, | 68.7 | Modification | Castello | RRM | DFCI |
| | BC017184.2 | | | | | |
| TRMT6 | BC001262.1 | 55.8 | Translation | Castello | Other | DFCI |
| TRMU | BC080631.1 | 25.2 | Other | | tRNA | DFCI |
| TRNAU1AP | BC039879.1 | 26.4 | Other | Baltz/Castello | RRM | DFCI |
| TRNAU1AP | BC000680.2 | 32.5 | Other | Baltz/Castello | RRM | DFCI |
| TROVE2 | BC038658.2 | 60.7 | Other | | Other | DFCI |
| TRUB2 | BC001457.2 | 36.7 | Modification | Baltz/Castello | Other | DFCI |
| TSR1 | BC125110.1 | 91.8 | Other | Castello | Other | DFCI |
| TSR1 | BC019090.2 | 75.0 | Other | Castello | Other | DFCI |
| TTYH1 | BC019358 | 49.1 | Other | | Other | In-house |
| TUFM | BC001633.1, | 49.9 | Translation | Castello | Other | DFCI |
| | BC010041.2 | | | | | |
| TUT1 | BC005013.1 | 57.9 | Other | | RRM | DFCI |
| TUT2 | uc010jaf.1 | 56.0 | Other | | Other | In-house |
| TUT3 | uc010vgo.2 | 75.8 | Other | | Other | In-house |
| TUT5 | uc003jdx.1 | 59.9 | Other | | Other | In-house |
| TUT7 | uc004aoq.3 | 171.2 | Other | | Other | In-house |
| TWF2 | BC016452.1 | 39.5 | Other | Castello | Other | DFCI |
| U2AF1 | BC001177.1, | 27.9 | Localization | Baltz/Castello | RRM | DFCI |
| | BC001923.1 | | | | | |
| U2AF1 | BC005915.1 | 19.8 | Localization | Baltz/Castello | RRM | DFCI |
| U2AF1L4 | BC021186.1 | 22.0 | Splicing | | RRM | DNASU |
| U2AF2 | BC008740.2 | 53.1 | Localization | Baltz/Castello | RRM | DFCI |
| UBAP2L | BC003170.1 | 114.5 | Other | Baltz/Castello | Other | DFCI |
| UBE2I | BC000427.2, | 18.0 | Other | Castello | Other | DFCI |
| | BC051289.1 | | | | | |
| UCHL5 | BC015521.1 | 37.5 | Other | Castello | Other | DFCI |
| USO1 | BC032654.1 | 107.8 | Other | Castello | Other | DFCI |
| USF32 | BC054344.1 | 44.7 | Other | | Other | DFCI |
| USP36 | BC038983.1 | 31.8 | Other | Castello | Other | DFCI |
| UTP11L | BC005182.1 | 30.4 | Other | Baltz/Castello | Other | DFCI |
| UTP14A | BC001149.1, | 88.0 | Other | Baltz/Castello | Other | DFCI |
| | BC009649.1, | | | | | |
| | BC014987.1 | | | | | |
| UTP15 | BC013064.1 | 32.4 | Other | Baltz/Castello | Other | DFCI |
| UTP23 | BC006955.1, | 28.4 | Other | Castello | Other | DFCI |
| | BC022441.1 | | | | | |
| UTP3 | BC004546.1 | 54.6 | Other | Baltz/Castello | Other | DFCI |
| WBSCR16 | BC007823 | 49.9 | Other | Baltz/Castello | Other | DNASU |
| WDR3 | BC058836.1 | 43 | Other | Castello | Other | DFCI |
| WDR33 | BC013990.2 | 38.3 | Other | Baltz | Other | DNASU |
| WDR36 | BC133025.1 | 105.3 | Other | Castello | Other | DFCI |
| WDR6 | BC002826.2 | 32.1 | Other | Castello | Other | DFCI |
| XPO1 | BC032847.2 | 123.4 | Localization | | Other | DFCI |
| XPO5 | BC000129.1 | 31.5 | Other | Baltz/Castello | Other | DFCI |
| XPO5 | BC009969.2 | 75.8 | Other | Baltz/Castello | Other | DFCI |
| XRCC6 | BC008343.1, | 69.8 | Other | Baltz/Castello | Other | DFCI |
| | BC012154.2 | | | | | |
| XRCC6 | BC010034.2, | 69.8 | Other | Baltz/Castello | Other | DFCI |
| | BC018259.2 | | | | | |
| XRN1 | NM_019001 | 194.1 | Stability | Baltz/Castello | Other | DNASU |
| XRN2 | BC006417.1 | 63.8 | Other | Baltz/Castello | Other | DFCI |
| YARS | BC001933.1, | 59.1 | Translation | Castello | tRNA | DFCI |
| | BC016689.1 | | | | | |
| YTHDC1 | BC041119.1 | 84.7 | Splicing | Baltz/Castello | Other | DFCI |
| YTHDF1 | BC050284.1 | 60.9 | Other | Baltz/Castello | Other | DFCI |
| YTHDF2 | BC002559.2 | 82.3 | Other | Baltz/Castello | Other | DFCI |
| YTHDF3 | BC052970.1 | 63.9 | Other | Baltz/Castello | Other | DFCI |
| YWHAE | BC000179.1, | 29.2 | Other | Castello | Other | DNASU |
| | BC001440 | | | | | |
| YWHAG | BC020963.2 | 28.3 | Other | | Other | DFCI |
| ZC3H11A | BC014268.2 | 89.1 | Localization | Baltz/Castello | ZnF-CCCH | DFCI |
| ZC3H14 | BC027807.2 | 34.9 | Other | Baltz/Castello | ZnF-CCCH | DFCI |
| ZC3H18 | BC050463.1 | 106.4 | Other | Castello | ZnF-CCCH | DFCI |
| ZC3H7A | BC012575.1 | 19.8 | Other | Baltz/Castello | ZnF-CCCH | DFCI |
| ZC3H8 | BC032001.1 | 34.3 | Other | Baltz/Castello | ZnF-CCCH | DFCI |
| ZC3HAV1 | BC040956.1 | 77.9 | Other | Baltz/Castello | Other | DFCI |
| ZCCHC11 | | 81.1 | Other | | Other | In-house |
| ZCCHC11 | | 81.1 | Other | | Other | In-house |
| ZCCHC11 | | 81.1 | Other | | Other | In-house |
| ZCCHC11 | BC131734.1 | 185.3 | Other | Baltz/Castello | zf-CCHC | DFCI |
| ZCCHC17 | BC007446.2, | 27.6 | Other | Baltz/Castello | S1 | DFCI |
| | BC050609.1 | | | | | |
| ZCCHC6 | AL832026 | 144.5 | Other | Baltz/Castello | zf-CCHC | DNASU |
| ZCCHC7 | BC034022.1 | 34.5 | Other | Castello | zf-CCHC | DFCI |
| ZCCHC7 | BC036940.1 | 62.9 | Other | Castello | zf-CCHC | DFCI |
| ZCCHC9 | BC014841.1 | 30.9 | Other | Castello | zf-CCHC | DFCI |
| ZCRB1 | BC022543.1 | 24.6 | Splicing | Baltz/Castello | RRM | DFCI |
| ZFC3H1 | BC073843.1 | 37.8 | Other | Castello | Zn-Finger | DFCI |
| ZFC3H1 | BC015679.2 | 39.1 | Other | Castello | Zn-Finger | DFCI |
| ZFP36 (TTP) | BC009693 | 34.0 | Other | | Other | In-house |
| ZFP36L1 | BC018340.1 | 36.3 | Stability | Castello | ZnF-CCCH | DFCI |
| ZFP36L2 | BC005010 | 51.6 | Stability | Castello | ZnF-CCCH | DNASU |
| ZGPAT | BC019338.1 | 54.7 | Other | | ZnF-CCCH | DFCI |
| ZMAT3 | BC002896.2 | 32.1 | Other | Castello | Zn-Finger | DFCI |
| ZNF9 | NM_003418 | 19.5 | Other | | Other | DNASU |
| ZRANB2 | BC039814.1 | 36.2 | Splicing | Baltz/Castello | Zn-Finger | DFCI |
| ZRSR1 | BC113599.1 | 67.6 | Other | | RRM | DFCI |
| ZRSR2 | BC113454.1, | 58.0 | Splicing | | RRM | DFCI |
| | BC113480.1 | | | | | |
| ZRSR2 | BC050451.11 | 58.0 | Splicing | | RRM | DFCI |
| ZYX | BC003743.2, | 61.3 | Other | Castello | Other | DFCI |
| | BC009360.2, | | | | | |
| | BC010031.2 | | | | | |
| AC004381.6 | AC004381.6 | 88.9 | Other | | Other | In-house |
| | (LOC81691 | | | | | |
| | exonuclease NEF-sp) | | | | | |

### Example 2

### Large-Scale Tethered Function Screen Assigns RBPs to Roles in RNA Stability and Translation

961 ORFs, representing 888 RBPs, were screened in triplicate. Two dual luciferase reporter systems were used as described above, and the FLAG expression construct was used as a negative control (FIG. 1D, left). The effect of RBP recruitment to the tethering reporter was calculated as the fold change in luciferase activity relative to FLAG control, after normalization of each to the untethered reporter (FIG. 1D, right). Supporting the validity of the screening approach, it was confirmed that the effect was not correlated with RBP size, indicating that steric hindrance is unlikely to account for these observations (FIG. 7D). The magnitude of the effect on reporter transcript abundance generally depended on the reporter systems (FIG. 7E) but for 97% of ORFs (961), both reporter systems agreed on the direction of regulation (FIG. 7F), indicating high reproducibility.

Candidates from each reporter assay were prioritized by using multiple t-tests at a threshold p<0.05 and calculated false discovery rates (FDR) for each comparison using the Benjamini, Krieger & Yekutieli procedure. 344 and 87 RBPs were identified with an estimated FDR <0.01 in Renilla and firefly reporters, respectively, of which 50 RBPs were recovered from both reporter contexts (FIG. 1E). In order to distinguish those RBPs that affect reporter mRNA stability from those regulating its translation, both luciferase transcripts were measured by reverse transcription quantitative PCR (rt-qPCR) for 35 RBPs of the 50 RBPs that significantly modulated luciferase activity in both reporter contexts. In general, the change in reporter translation levels was positively correlated with changes in reporter transcript levels (FIG. 1F). Among the strongest candidate negative regulators were RBP components of both deadenylation-dependent and -independent exonuclease decay pathways, including ZFP36, as well as members of the CCR4-NOT deadenylase complex (CNOT2, CNOT4, CNOT7, TOB1, and TOB2), the 3′-to-5′ exonuclease PARN, and the decapping activator DDX6, which is recruited to the 5′ cap via interaction with the CCR4-Not complex. As another positive control, it was also confirmed that YTHDF2, a member of the YTH domain family of N6-methyladenosine binding proteins, which recruit target RNAs to degradation bodies, exerts a negative effect on target mRNA levels. The results of the screen also confirmed several known negative regulators of translation, such as NANOS3 specific to germ cells, and CPEB4, which binds polyadenylation elements in the 3′ UTR and negatively regulates translation initiation by interacting with the translation initiation factor eIF3. Interestingly, EIF2S2, with roles in promoting translation initiation, emerged as positive regulator of translation when recruited to the 3′ UTR. It was speculated that recruitment of this protein to the 3′ UTR brings it into proximity to the mRNA cap and 5′UTR, similar to DDX6 and CPEB4 and consistent with the closed-loop model of translation (FIG. 1G).

To verify these RBPs hits are not false positive in the large screen assay, reporter protein and transcript level changes were re-confirmed by luciferase assay and qRT-PCR and chose 14 RBPs with significant effects (8 candidate stabilizers and 6 candidate destabilizers) for further investigation. Focus was put on RBPs with known roles in RNA stability and translation but where transcriptome-wide binding sites and preferences have not been described (e.g. CNOT7, DDX6, NANOS3, TOB1/2, MEX3C) and RBPs for which such roles are not known (e.g. UBAP2L, AIMP1, MTDH, IFTI2) (FIGS. 1H-1I).

In summary, the screen revealed RBPs previously annotated to be implicated in metabolic processes, cell cycle, cell differentiation (BOLL, DAZ2, DAZ4, DAZAPI, NANOS3), stress granule regulators (UBAP2L), translation machinery (EIF2S2, LARP1, PABPCI, CPEB4), ER proteins (SRPR), and heat shock proteins (HSPB1). Eight annotated splicing factors (CLK3, CPSF5, PLRG1, PRPF3, RBFOX1, F3B3S, NRNP27, and SNRPA) and three nuclear export complex proteins (HNRNPD, THOC1, and YWHAE) were identified (FIGS. 1J-1L).

### Example 3

### Enhanced CLIP Identifies Endogenous RNA Targets of Candidate Stabilizers and Destabilizers

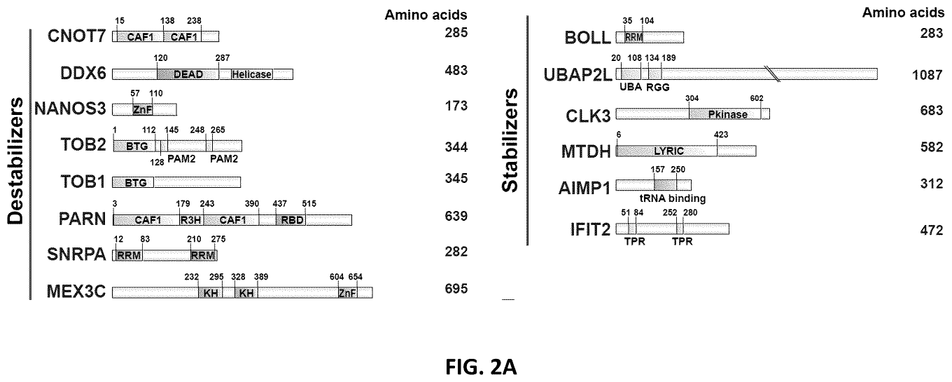

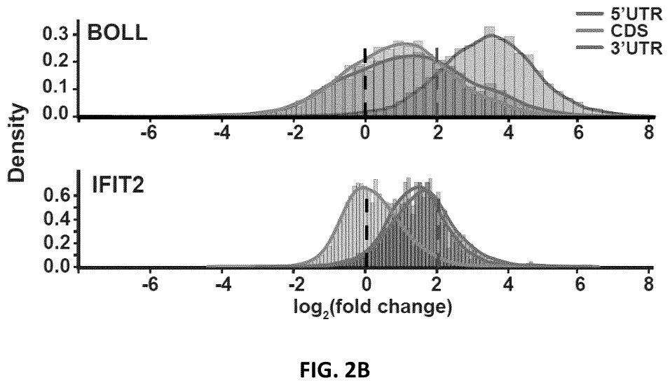

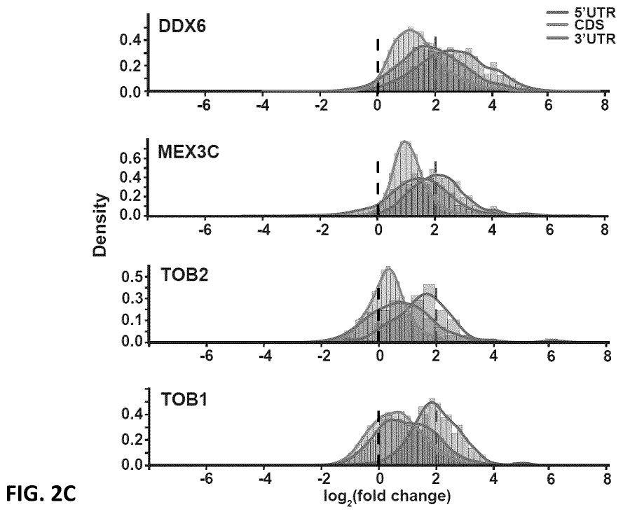

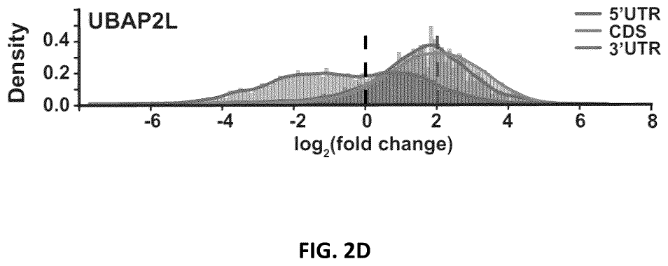

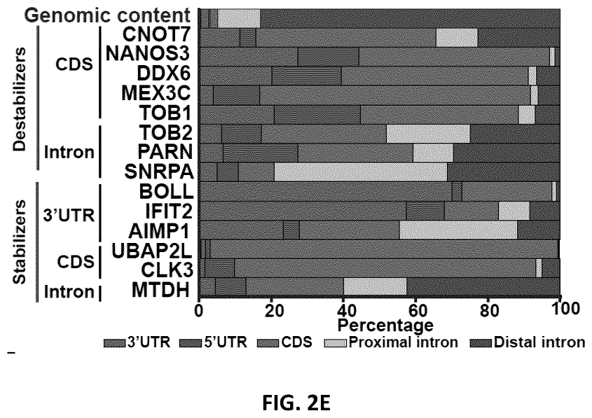

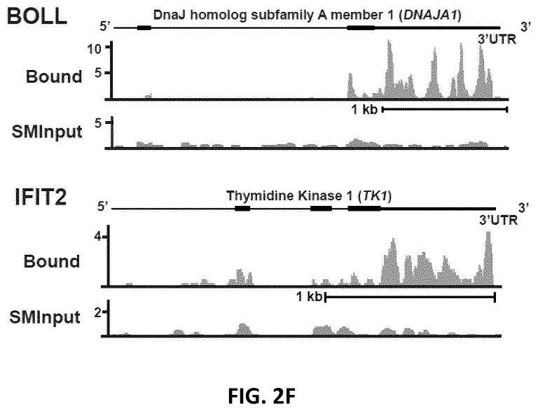

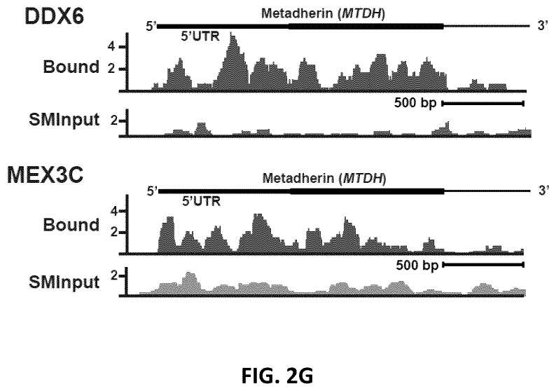

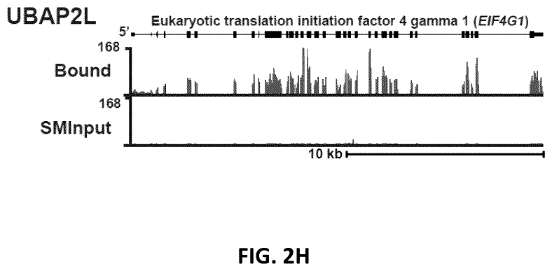

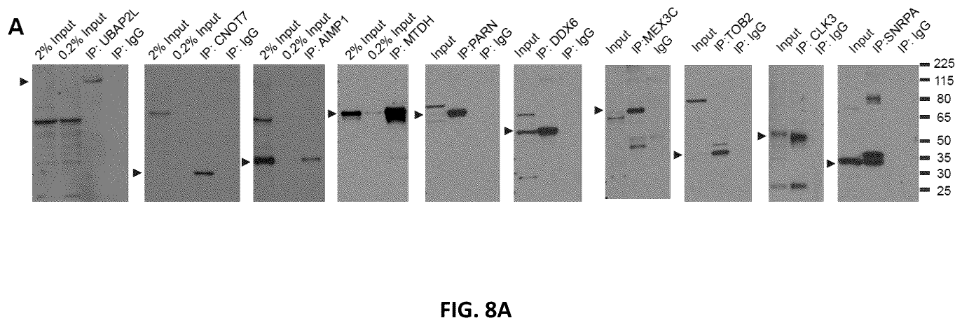

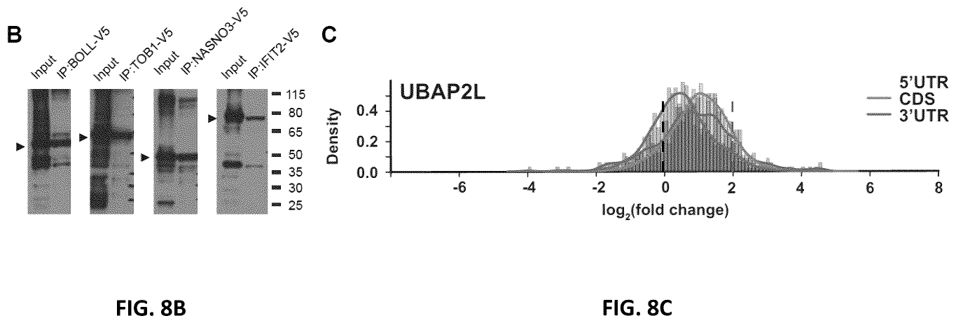

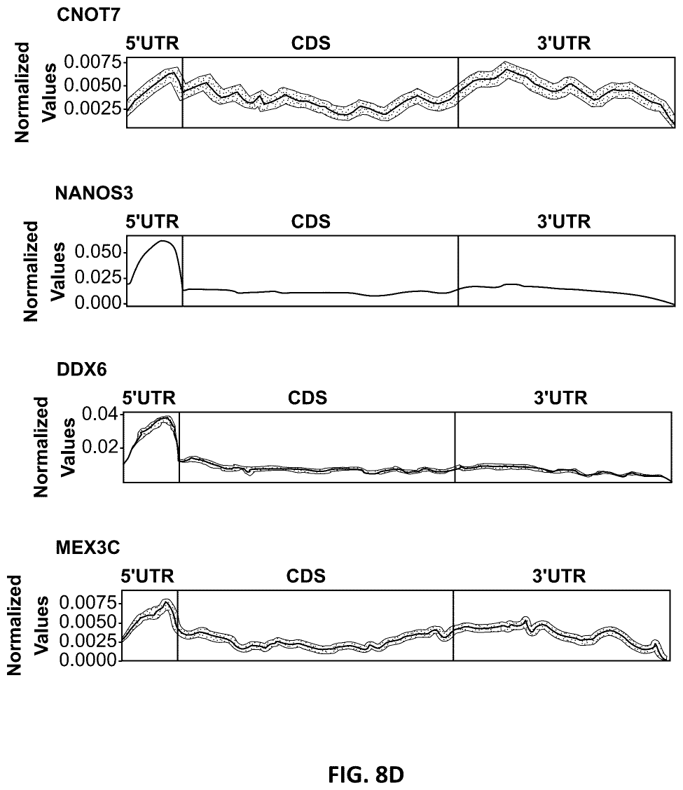

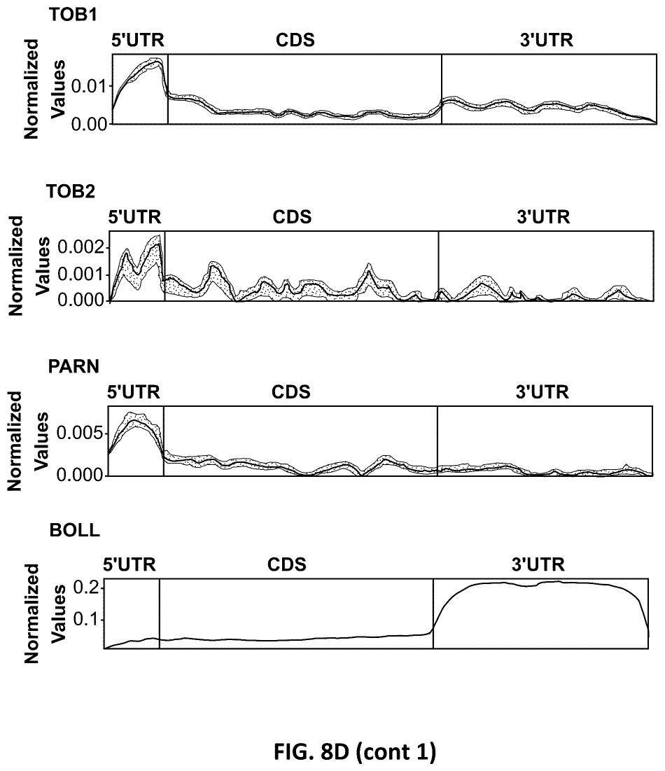

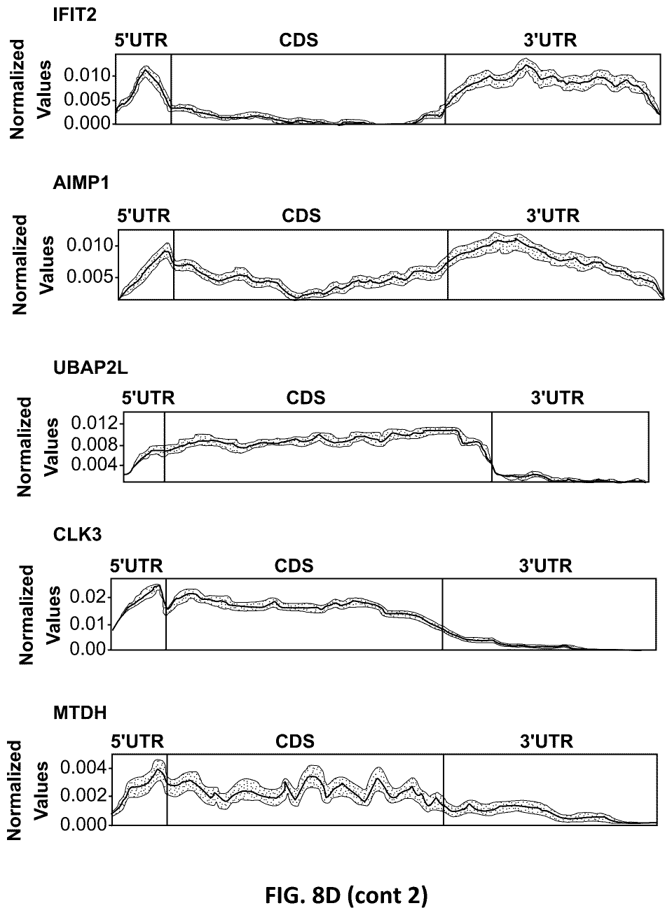

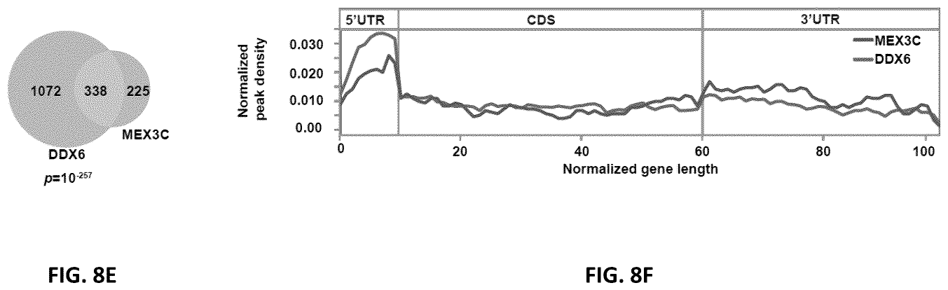

In order to begin elucidating the physiological functions of candidate RBP regulators (FIG. 2A), their endogenous mRNA targets and their transcriptome-wide binding sites were investigated using enhanced cross-linking immunoprecipitation followed by sequencing (eCLIP). HEK293T cells were subjected to UV-crosslinking, lysis and RNA fragmentation, and protein-RNA complexes were immunoprecipitated using validated RBP-specific antibodies (FIG. 8A). Also, cells were transiently transfected with plasmids expressing V5-tagged fusions of those candidate RBPs which are not expressed in HEK293T cells or do not have RBP-specific antibodies (FIG. 8B). In total, cCLIP datasets for 14 candidate proteins were generated, each consisting of an RBP eCLIP (IP) library and a paired size-matched input (SMInput) library. Libraries were sequenced to at least 15 M (million) reads (average of 24 M), of which at least 1 M (average of 5 M) mapped uniquely to the human genome.

Next, transcript binding region specificities were determined using two distinct metrics, namely read density enrichment and binding cluster enrichment. Read density enrichment within 5′ and 3′UTRs and coding regions (CDS) of annotated protein coding genes were computed by the fold enrichment in the IPs normalized to their paired SMInputs. To illustrate, BOLL, a germ-cell specific RBP with some documented roles in mRNA stabilization and translation enhancer activity, displayed a strong preference for 3′UTR association (FIG. 2B). Surprisingly, IFIT2 (Interferon Induced Protein With Tetratricopeptide Repeats 2), which is known to inhibit expression of viral messenger RNAs, robustly displays a strong 3′UTR preference in human mRNAs. The helicase DDX6 was enriched for binding within 5′UTRs, consistent with its role in the assembly of the decapping complex. A novel candidate 170 MEX3C, an RNA-binding E3 ubiquitin ligase that associates with the CCR4-NOT deadenylation complex to ubiquitinate CNOT7, unexpectedly exhibited preferential binding to 5′UTRs. Similarly, TOB family members TOBI and TOB2, which recruit the catalytic subunits of the CCR4-NOT deadenylasc complex to target mRNAs, showed a surprising preference for 5′UTRs, suggesting unexpected roles for this family of proteins (FIG. 2C). Distinct from all these RBPs, UBAP2L (Ubiquitin-associated protein 2-like) showed strong enrichment across exons, especially in CDS, and 5′ UTR (FIG. 2D; FIG. 8C).

To identify binding sites at higher resolution, binding clusters were discovered by the CLIPper algorithm. Cluster enrichment was computed by calculating the ratio of read densities between IPs and SMInputs within a cluster and significant clusters were defined as p≤10⁻³ (Fisher's exact test for read numbers <5; ω² test for read numbers ≥5) and ≥4-fold enriched over SMInput. The significant clusters were generally located within the same enriched regions from the lower resolution gene region analysis (FIG. 2E; FIG. 8D). For example, the clusters for BOLL and IFIT2 were most enriched in 3′UTRs (FIG. 2F). Interestingly, DDX6's and MEX3C's target genes (FIG. 8E) and binding clusters (FIG. 2G; FIG. 8F) strongly overlap, suggesting that both proteins may be functionally linked and may act on the same mRNA targets. In contrast to the other candidate RBPs, the UBAP2L clusters were dispersed across exonic regions, rather than present as discrete binding sites (FIG. 2H). Overall, the analyses not only revealed previously unrecognized binding maps and preferences for RBPs known to affect mRNA stability and translation (CNOT7, DDX6), but also revealed novel RNA interactomes of candidate RBPs.

### Example 4

### Integration of eCLIP and RNA-seq Data Defines Regulatory Classes of RBPs and Transcripts

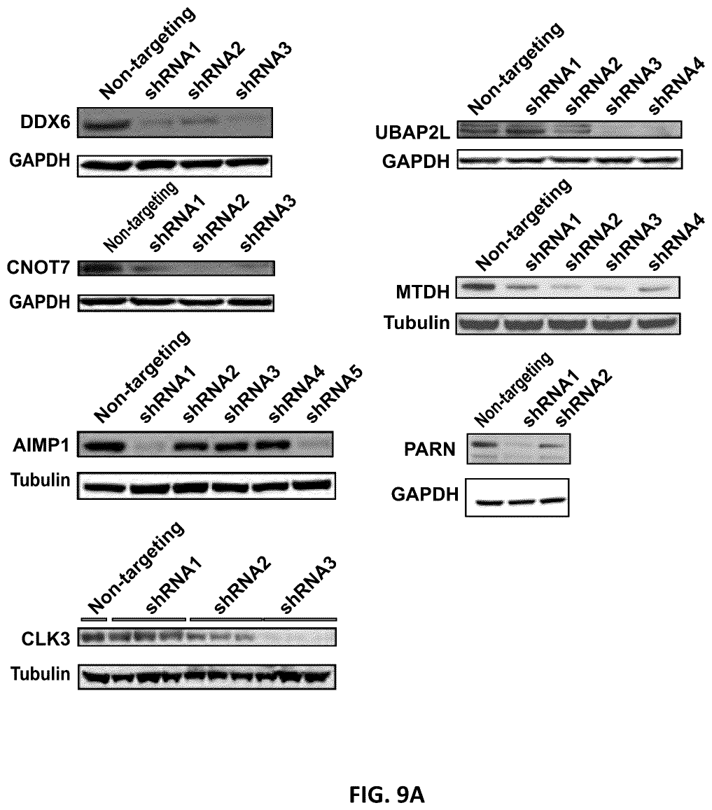

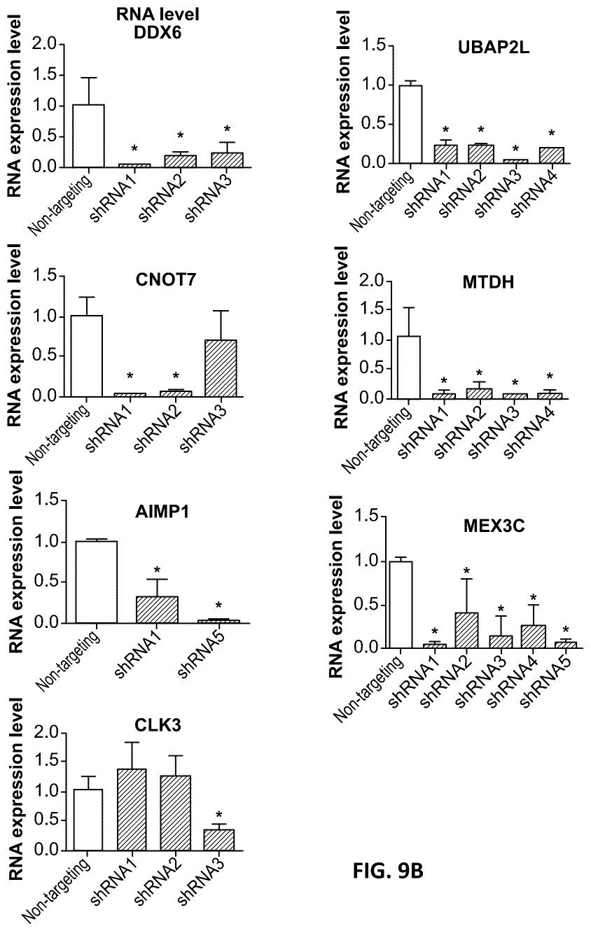

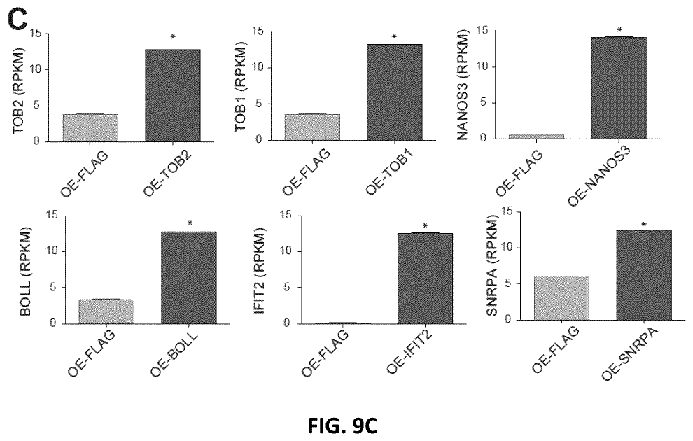

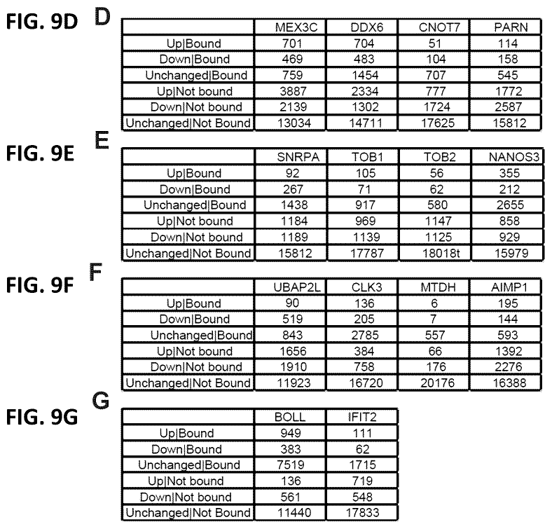

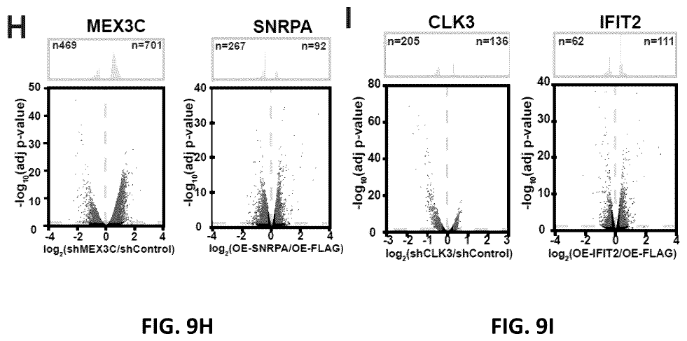

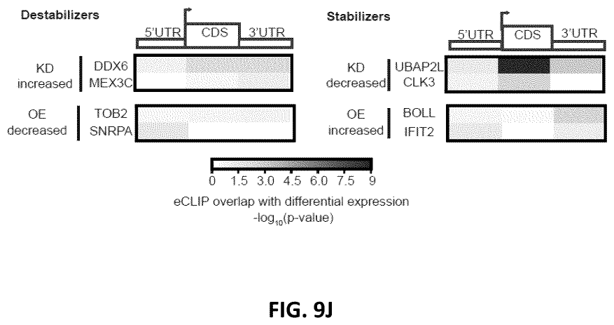

To gain insight into how the candidate RBPs affect transcriptome-wide mRNA levels, they were depleted or exogenously expressed in HEK293T cells and RNA-seq analysis was performed. Specifically, RBPs were either depleted by lentiviral transduction of short-hairpin RNAs (shRNAs) (FIGS. 9A-9B), or ectopically expressed ORFs of those candidate RBPs which are not expressed in HEK293T cells or which do not have RBP-specific shRNAs (FIG. 9C). For each RBP, either two independent transductions of two different targeting shRNAs and two non-targeting shRNAs were performed, or two independent transfections with a plasmid directing expression of the RBP as a V5-tagged fusion were performed, with the FLAG construct as a control. PolyA+RNA was selected, sequencing libraries were prepared and sequenced to a depth of >32 (or >26 uniquely mapped)×10⁶ reads.

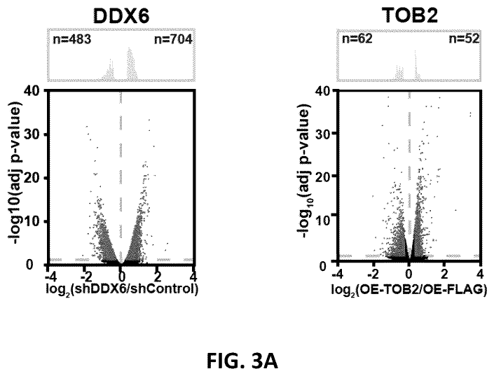

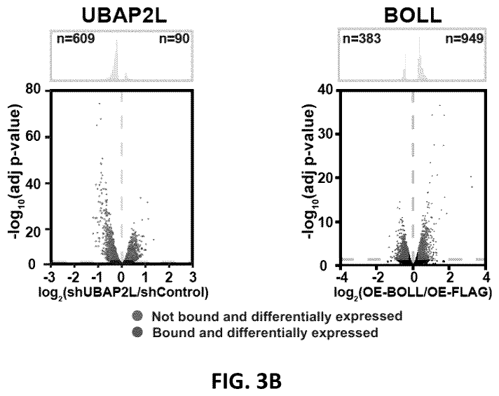

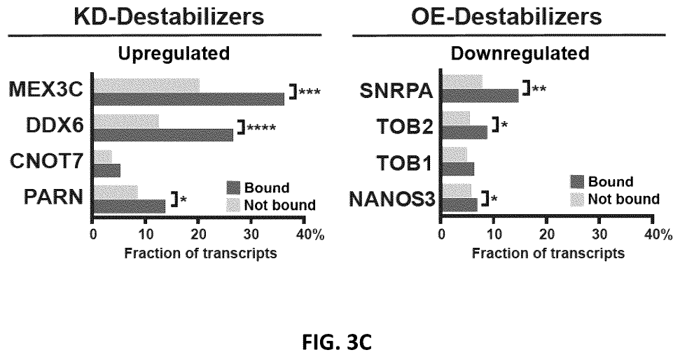

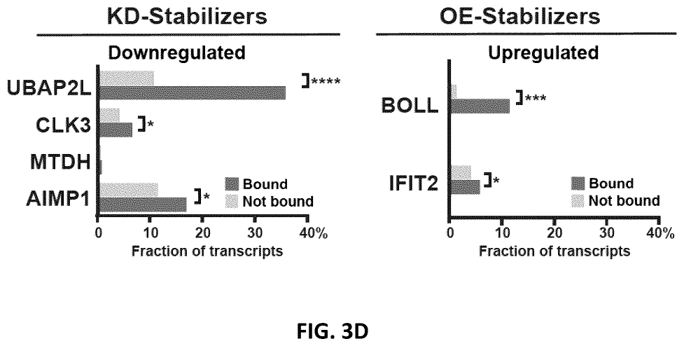

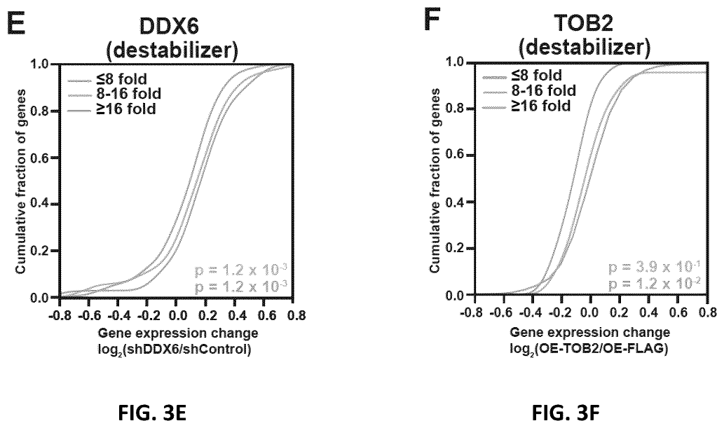

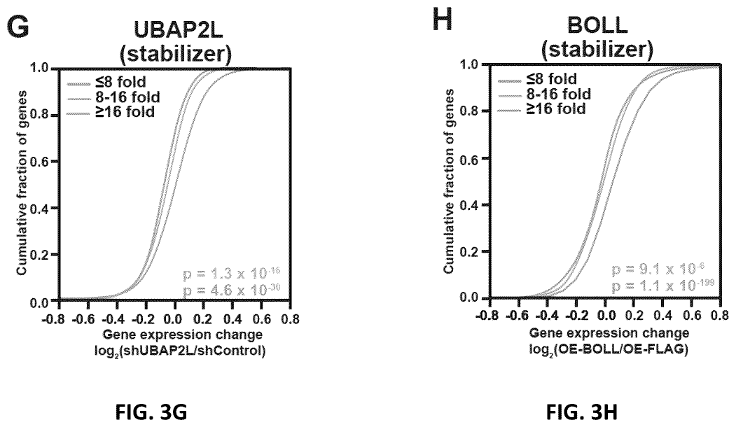

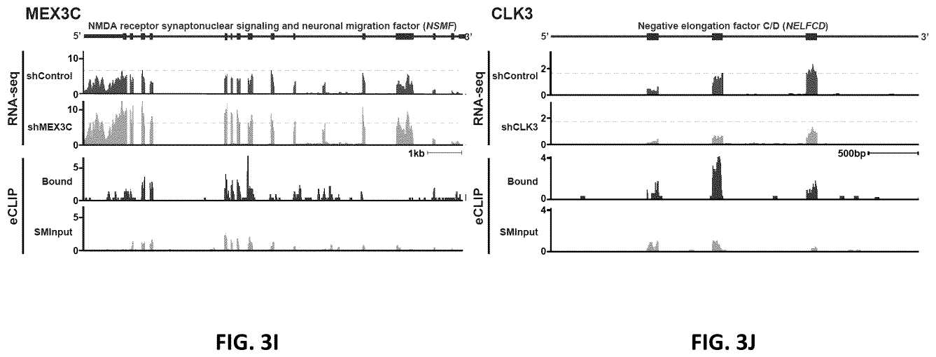

To assess the effect of a candidate RBP on transcript levels, the number of significantly up-or down-regulated genes were measured upon knockdown or overexpression (FIGS. 9D-9G). In general, the manipulations of RBP levels resulted in a largely unperturbed population of transcripts, typically 80% at threshold of statistical significance [≥1.23-fold, false discovery rate (FDR)-corrected p≤0.05 versus non-targeting shRNA or FLAG control]. This indicates that the candidate RBPs affect specific sets of target transcripts, instead of having effects on global transcript stability. When only considering those transcripts that were bound by the respective RBP, as measured by eCLIP (≥1 significantly enriched cluster per transcript), higher numbers of targets were observed that change in the direction anticipated by the tethering assays, than in the opposite direction, for candidate destabilizers, MEX3C, DDX6, SNRPA, and TOB2 (FIG. 3A; FIG. 9H) and candidate stabilizers UBAP2L, CLK3, BOLL, and IFIT2 (FIG. 3B; FIG. 9I). In other words, knockdown of destabilizers led to more up-regulated genes, whereas overexpression of destabilizers led to more down-regulated genes. Expectedly, reciprocal effects are observed in the alterations of stabilizing RBPs.

It was also confirmed that the fraction of bound targets in the genes changing in the anticipated direction was statistically significantly enriched relative to unbound targets (FIGS. 3C-3D). In fact, significant correlation was observed between different eCLIP cluster fold enrichments IP over SMInput and change in transcript levels, for both candidate destabilizers (e.g. DDX6 and TOB2; FIGS. 3E-3F) and candidate stabilizers (e.g. UBAP2L and BOLL; FIGS. 3G-3H). This indicates that the candidate RBPs directly engage hundreds of previously unknown target endogenous mRNAs to affect transcript levels in the predicted direction. For example, knockdown of the destabilizer MEX3C increased transcript levels of NSMF mRNA, a MEX3C-bound transcript (FIG. 3I). Conversely, depletion of the stabilizer CLK3 reduced the abundance of its target NELFCD mRNA (FIG. 3J). Interestingly, when it was further evaluated which genic regions bound by the RBP are most correlated with transcript levels, UBAP2L binding within CDS was the most enriched (FIG. 9J). In general, it was concluded that the majority of the candidate RBPs affect mRNA levels of their endogenous RNA targets, in agreement with the tethering results.

### Example 5

### UBAP2L Increases mRNA Polysome Association and Promotes Translation

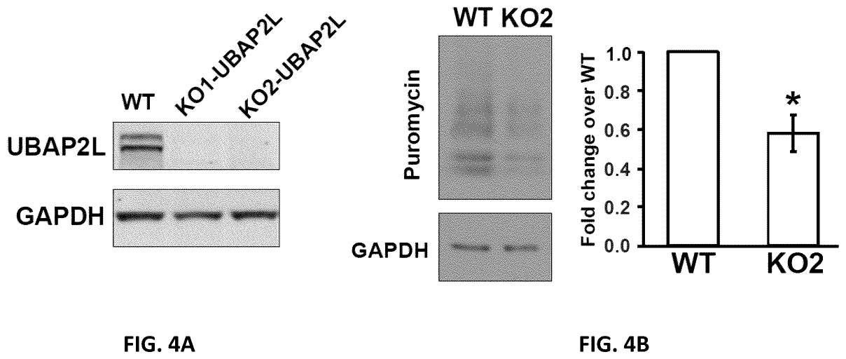

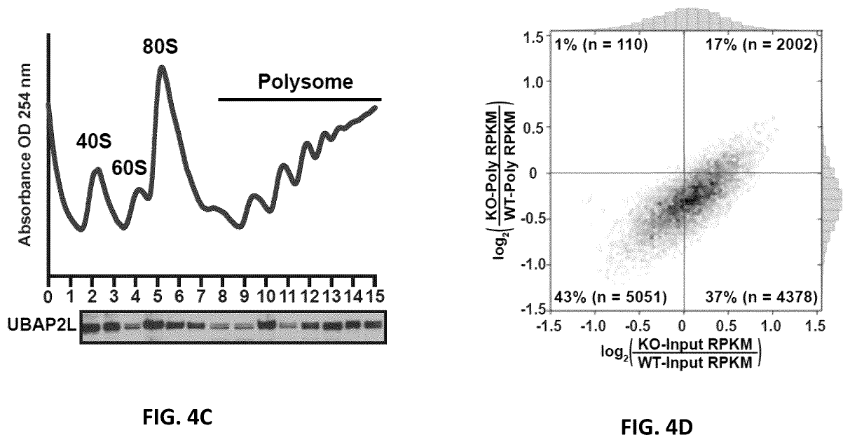

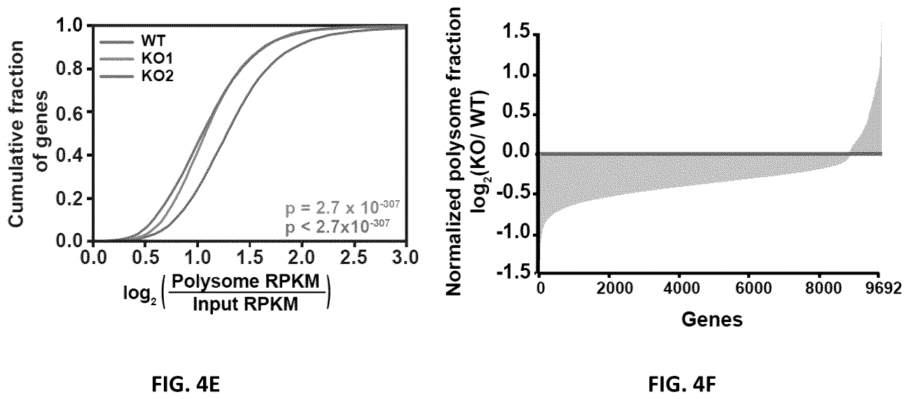

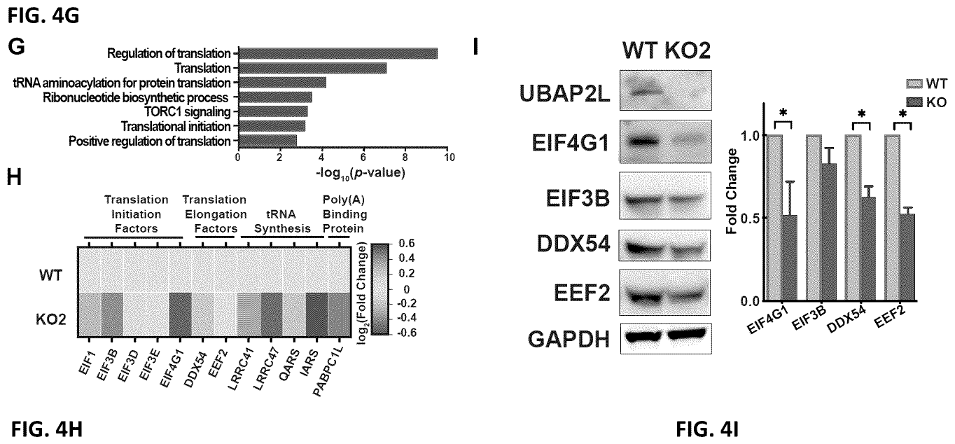

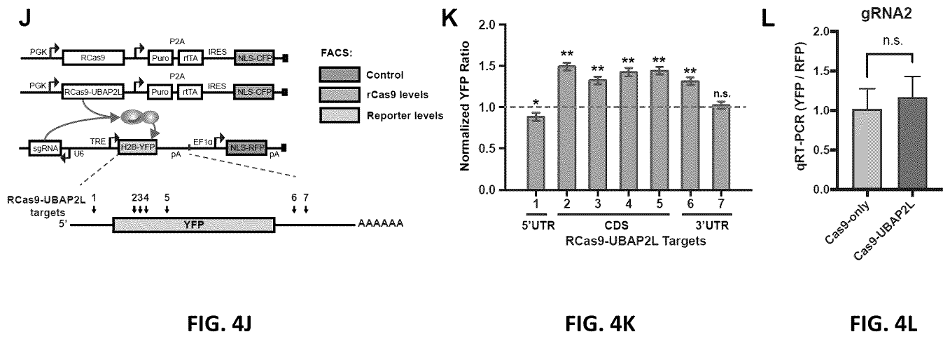

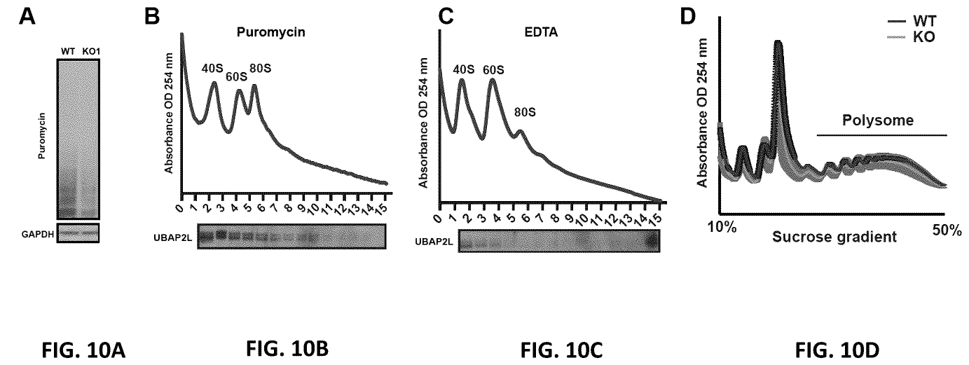

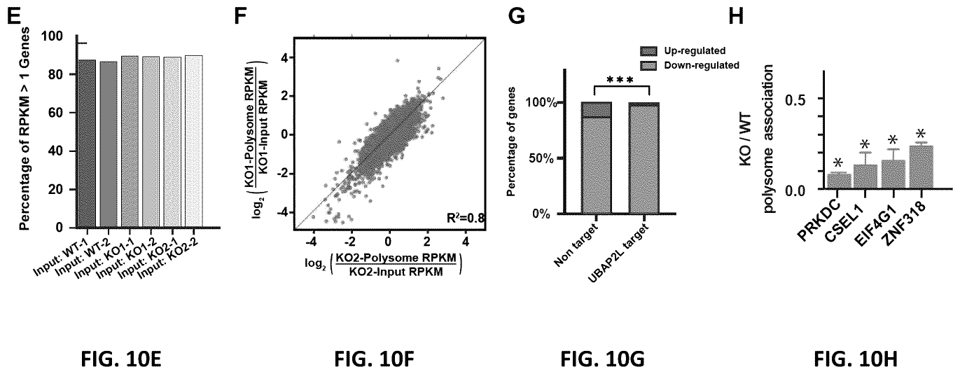

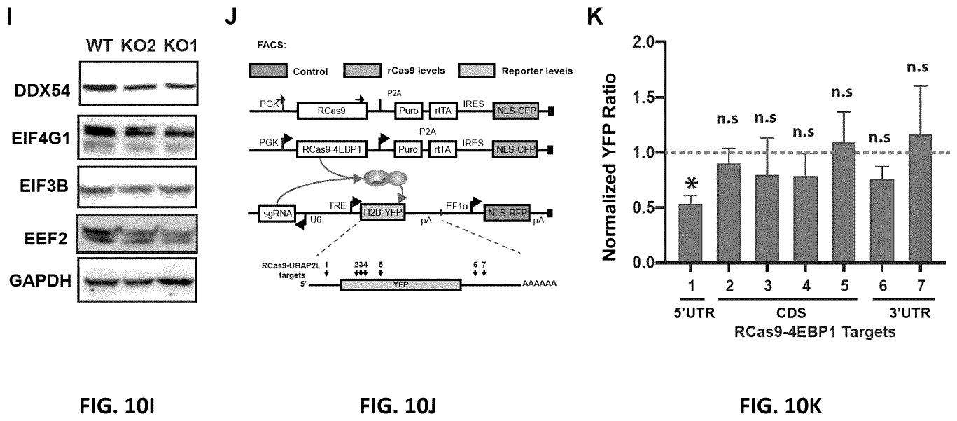

Among the 13 candidates that were analyzed, UBAP2L had the highest CDS read density enrichment (FIG. 2D and FIG. 9J), suggesting a direct role in translation. However, such a function for UBAP2L had not been described. Global protein synthesis rates were measured in cells lacking UBAP2L with the SUNSET assay, which uses incorporation of puromycin (a structural analog of aminoacyl-transfer RNA) to label newly synthesized proteins. HEK293T cells biallelically deleted for UBAP2L by CRISPR/Cas9-mediated genome editing showed a ˜40% reduction in protein synthesis (FIGS. 4A-4B; FIG. 10A), indicating that UBAP2L promotes global translation. Next, sucrose gradient centrifugation of HEK293T lysates was performed to examine the association of UBAP2L with ribosomes. UBAP2L from HEK293T cell lysates co-fractionated with monosomes and polysomes on sucrose gradients, suggesting a role for UBAP2L in translation (FIG. 4C). In order to rule out the possibility that this observation is due to the presence of UBAP2L in non-ribosomal complexes of similar buoyant density, cells were treated with puromycin to release polysomes from transcripts. Puromycin treatment led to accumulation of 80S monosomes, as expected, and levels of UBAP2L in fractions corresponding to polysomes were strongly reduced (FIG. 10B). Cell lysates were also treated with EDTA to disassemble 80S monosomes into 40S and 60S ribosomal subunits and found that, similarly, UBAP2L was depleted from fractions corresponding to monosomes (FIG. 10C). These results strongly suggest that UBAP2L directly interacts with translating ribosomes.

To identify specific transcripts subject to UBAP2L-mediated translational regulation, polysome profiling was performed in cell lysates from two independent UBAP2L knockout clonal isolates and from two control samples (FIG. 10D). From two independent fractionations per line, polyA⁺ mRNA was isolated from a portion of the input lysates and from pooled polysome fractions, and RNA-seq libraries were prepared and sequenced. All transcripts with RPKM≥1 in inputs were considered (FIG. 10E). It was found that UBAP2L knockout resulted in a larger number of transcripts with changes in pooled polysome fractions compared to changes in input RNA abundance (FIG. 4D), suggesting that UBAP2L predominantly acts at the translational level. As a measure of ribosome association, the ratio of transcript RPKMs in polysome pools over input for all transcripts was computed. A significant decrease was found (p<10-307; Mann-Whitney U test, two-tailed) in mean transcript polysome-enrichment in both UBAP2L knockout lines compared to the controls (FIG. 4E). Replicate analyses showed excellent correlation between the cell lines (FIG. 10F). When isolated those genes that changed in the same direction in both knockout lines were isolated, it was found that overall nearly 10-fold more transcripts were reduced in translation (90.6%; n=8,784) than enhanced (9.4%; n=908) (FIG. 4F). Even more strikingly, 99% of the 1,425 UBAP2L target transcripts, identified by cCLIP, showed significant down-regulation in polysome association upon UBAP2L knockout (FIG. 10G). A subset of target transcripts were also measured by quantitative RT-PCR, which confirmed the magnitude of translational downregulation (FIG. 10H).

To investigate how depletion of UBAP2L affected global translation, the gene function attributes of UBAP2L direct targets were evaluated where a significant enrichment (FDR <0.05) was observe in protein translation and ribosome biogenesis terms by Gene ontology (GO) analysis (FIG. 4G). It was also revealed that UBAP2L depletion decreased polysome association on mRNAs encoding translation initiation factors, elongation factors, tRNA synthesis proteins, and poly (A) binding proteins (FIG. 4H). In fact, western blot analysis of these UBAP2L targets confirmed decreased protein levels of translation and elongation factors, such as Eukaryotic Translation Initiation Factor 4 Gamma 1 (EIF4G1), DEAD-Box Helicase 54 (DDX54), and Eukaryotic Translation Elongation Factor 2 (EEF2) in cells lacking UBAP2L (FIG. 4I and FIG. 10I). Taken together, these results suggest that UBAP2L enhances translation by directly binding mRNA substrates and also increasing translation of genes involved in global protein synthesis.

### Example 6

### Programmable RNA-Targeting CRISPR-Mediated Recruitment of UBAP2L Promotes Translation

In order to assess the dependence of UBAP2L-mediated translational regulation on direct binding to its target mRNA, a FACS-based reporter assay was employed using UBAP2L fused to RNA-targeting RCas9 (RCas9) (FIG. 4J). As a control, the assay was performed with RCas9-fused 4EBP1, an inhibitor of translational initiation (FIG. 10J). HEK293T cell lines expressing a RCas9-UBAP2L fusion, RCas9-4EBPI fusion, or Cas9 only were derived via transposase-mediated piggyBAC genomic integration of plasmid constructs. A second construct harboring a reporter that stably expresses RFP transcripts not regulated by RCas9, a guide RNA, and tetracycline-inducible YFP transcripts was then transfected with the guide RNA target sequences. 7 different guide RNAs were designed, targeting locations across the YFP transcript (5′ UTR, CDS, and 3′UTR), and a non-targeting guide RNA. Post transcriptional regulation was then measured as changes in the normalized YFP/RFP fluorescence ratio between Cas9-fusion and Cas9 only cells by using analytical flow cytometry. Due to the random nature of piggyBAC-mediated integration in terms of construct integration sites and numbers, regulation for various rCas9 construct levels (CFP) and reporter construct levels (RFP) can be quantified across thousands of data points (cells). With this highly sensitive and quantitative assay, it was observed that the extent of the most strongly enhanced effect of UBAP2L on YFP reporter expression was dependent on UBAP2L directed to targeting sites within the 3′UTR and coding regions (FIG. 4K). In contrast, significant 4EBP1-mediated reporter repression was only observed when 4EBP1 was targeted to the 5′ UTR, as expected (FIG. 10K). Normalized YFP mRNA levels were not significantly different between RCas9-UBAP2L and RCas9 expressing cells transfected with gRNA 2 (which elicited the strongest increase) (FIG. 4L). These results indicate 305 that UBAP2L's positive effect on reporter expression was not due to upregulation of reporter mRNA. The UBAP2L-RCas9 results indicate a programmable means to enhance translation and further corroborate the observations from eCLIP and tethering in another orthogonal manner.

### Example 7

### UBAP2L Binds to RNA Via the RGG Domain and Crosslinks to the Expansion Segments of the Ribosome

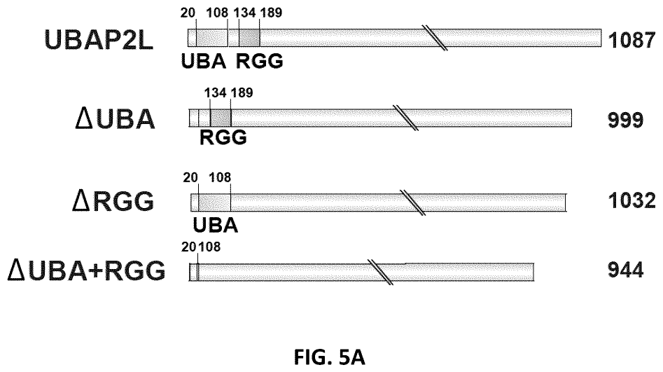

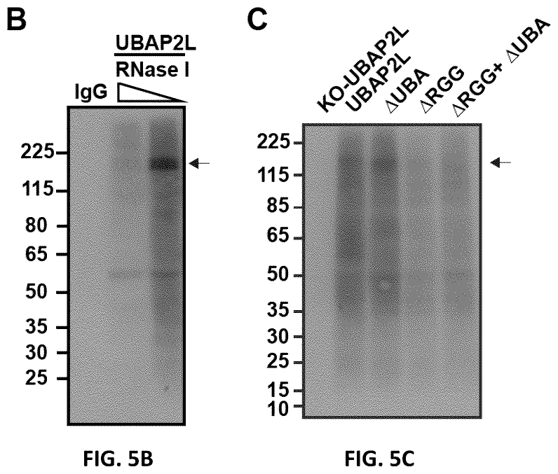

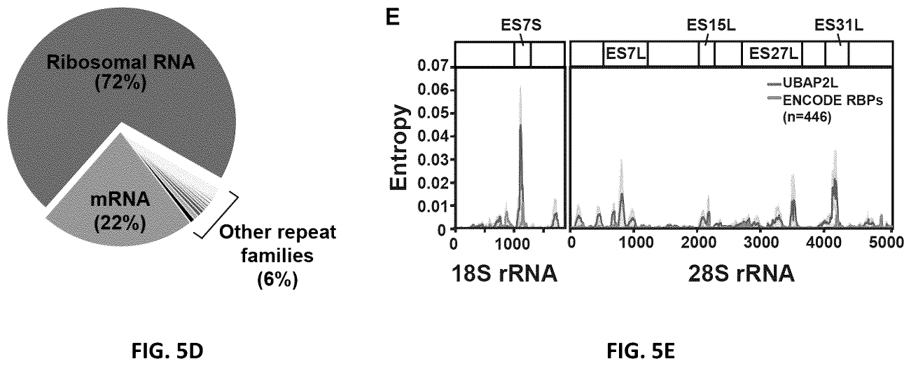

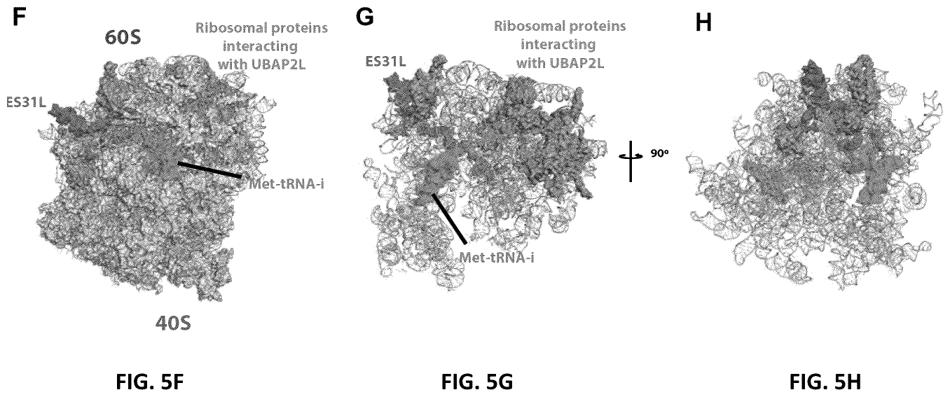

To gain molecular insight into the mechanisms by which UBAP2L enhances mRNA translation, it was determined which protein domains mediate UBAP2L's interaction with RNA. UBAP2L is predicted to contain only two structured domains: a ubiquitin-associated (UBA) domain and an Arg-Gly-Gly repeat (RGG) domain, a common RNA and protein binding domain. Using inducible lentiviral vectors, UBAP2L was expressed, or truncated versions lacking the UBA domain (DUBA), the RGG domain (DRGG), or both (FIG. 5A), in UBAP2L knockout HEK293T cells. Then, UV-crosslinking, immunoprecipitation, RNA fragmentation and radiolabeling was performed to visualize RNA bound to UBAP2L (FIG. 5B). Deletion of the RGG domain resulted in dramatically reduced recovery of RNA, indicating that the interaction between UBAP2L and RNA is mainly mediated by the RGG domain (FIG. 5C).

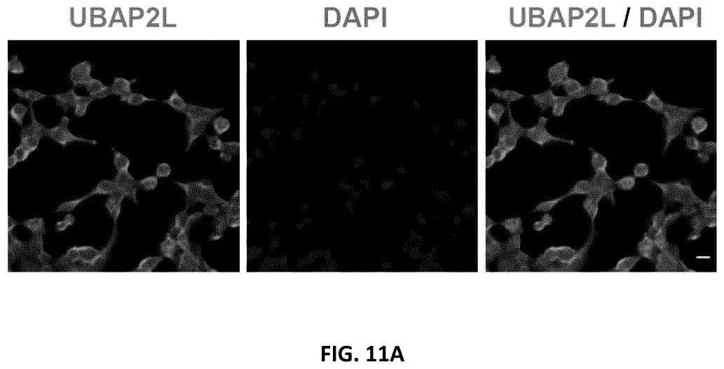

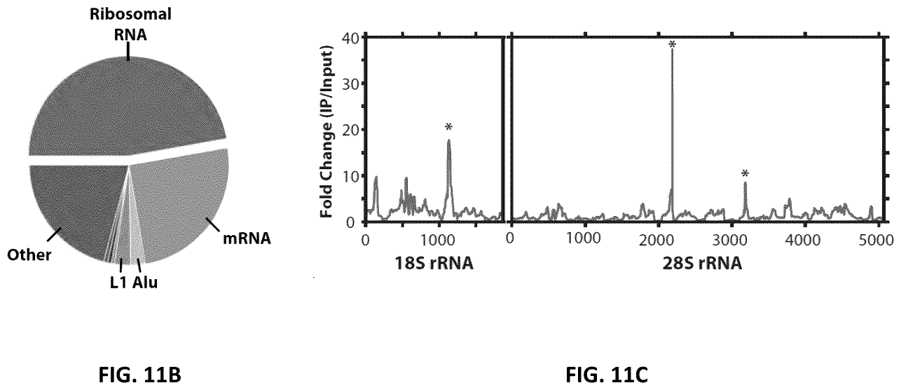

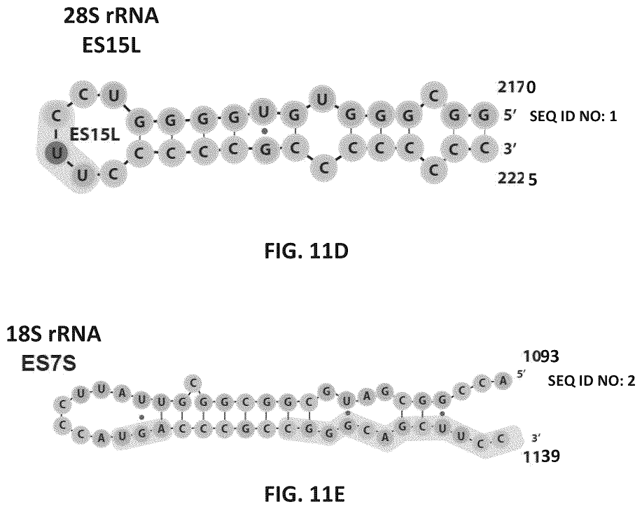

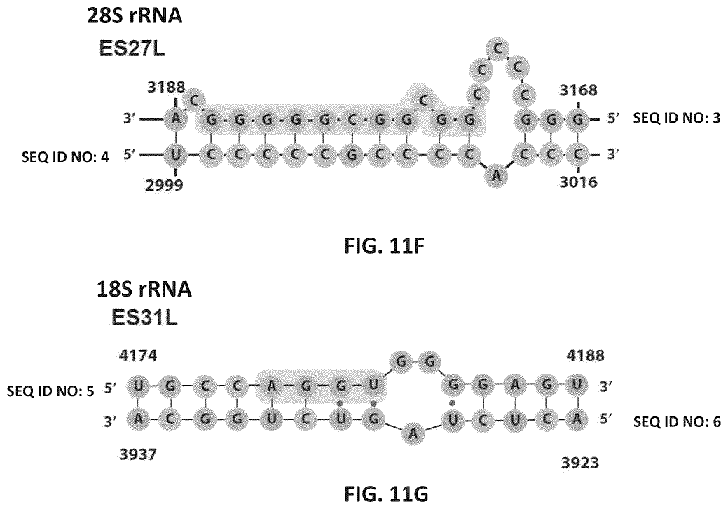

Given that UBAP2L cofractionated with monosomes and polysomes in sucrose gradients, it was reasoned that UBAP2L may interact directly with functional ribosomes. It was confirmed that UBAP2L is localized to the cytoplasm (FIG. 11A). Two UBAP2L eCLIP datasets were next examined using a repeat-family centric mapping strategy, which maps reads to consensus transcripts from repetitive and recurrent genomic loci, including ribosomal RNA (rRNA) genes. Remarkably, reads from rRNAs constituted the largest fraction (47%-72%) in both replicates, while mRNA reads totaled 22-25% (FIG. 5D; FIG. 11B). Closer inspection showed that reads were most highly enriched over SMInput at the expansion segments(ES) 15L, 27L of 28S rRNA, and ES7S of 18S rRNA (FIGS. 11C-11F), which are located at the solvent-exposed surface of ribosomes and are thought to engage with RBPs and mRNAs to modulate translation. As a further measure of the confidence of fold-enrichment, an information theoretic metric was utilized, relative entropy, which scales each enrichment with the strength of evidence (i.e. read depth) at each peak. It was confirmed that the peaks at ES15L, ES27L and ES7S (and an additional peak at ES31L) contained high information content (FIG. 5E; FIG. 11G). In contrast, the mean of 446 other RBPs shows very limited information content as a reflection of their specificity for binding the rRNAs. These cross-linking results indicate that UBAP2L directly interacts with ribosomes. This is consistent with a previous UBAP2L IP-mass spectrometry study that recovered peptides from 15 ribosomal proteins, further supporting a UBAP2L-ribosome interaction.

To assess the spatial arrangement of UBAP2L and the ribosome, these interactions were mapped onto the cryo-electron microscopy structure of the mammalian ribosome. The top ribosomal proteins that co-immunoprecipitate with UBAP2L cluster in the 60S subunit (FIG. 5F). In addition, ES31L, which is highly enriched for UBAP2L binding, lies close to the region of the 60S subunit, which is normally occupied by tRNA in the peptidyl site (P site) during protein synthesis (FIGS. 5G-5H). Collectively, these data support a model in which UBAP2L's function is associated with interactions with the ribosome.

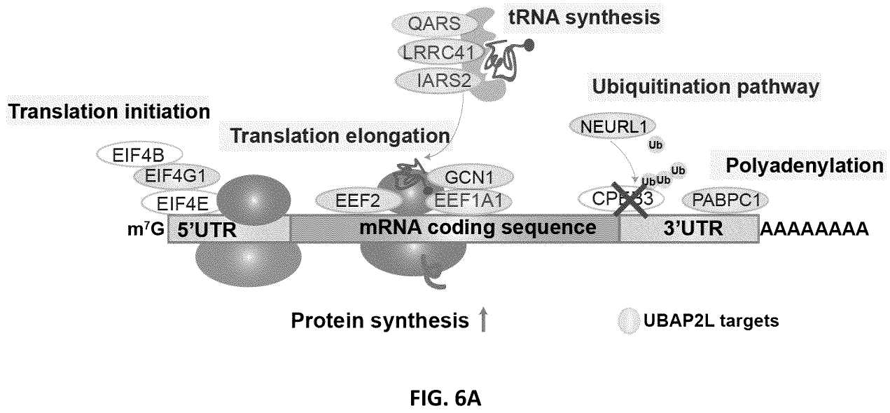

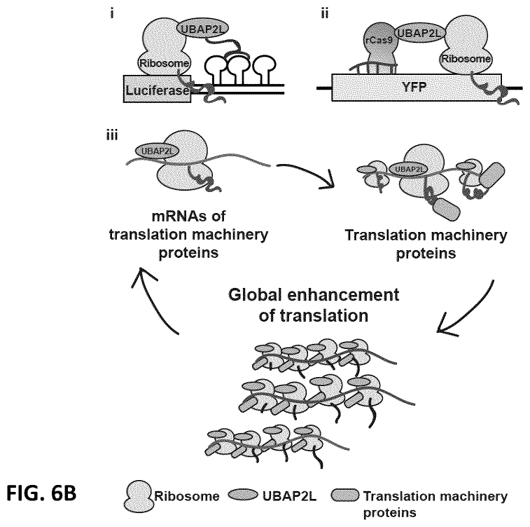

Furthermore, the transcriptome-wide analyses reveal that UBAP2L affects a significant number of mRNA targets, wherein mRNAs targeted by UBAP2L are themselves enriched for central regulators of translation, and protein synthesis (FIG. 6A), revealing a role for UBAP2L in modulating protein homeostasis in a global manner. The current working model proposes that UBAP2L is dynamically recruited to translating ribosome-mRNP complexes to enhance translation on many targets, including translational regulators to affect global protein synthesis (FIG. 6B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-20. (canceled)

21. A method of modulating translation of a target RNA in a cell comprising:
(a) assembling a modulation unit, wherein the modulation unit comprises:
(i) an RNA binding protein (RBP) comprising BTG1, CNOT2, CNOT4, CNOT7, CPSF5, DDX6, EWSR1, FUBP1, hnRNPAO, hnRNPC1/2, MEX3C, NANOS1, NANOS2, NOP56, PARN, PRR3, RBM14, RBM7, RPS6, SAMD4A, SNRPA, SRSF11, TOB1, TOB2, UTP11L, YTHDF2, ZC3H18, ZCCHC11, ZFP36, ZFP36L1, ZFP36L2, ABT1, AC004381.6, AIMP1, ALDH18A1, ANXA2, APOBEC3F, ASCC1, ATP5C1, BCCIP, BOLL, BYSL, BZW1, CELF5, CLK1, CLK2, CPSF1, DAZ2, DAZ3, DAZ4, DCN, DDX1, DDX19B, DDX20, DDX39A, DMPK, EEF1A1, EIF3G, ERAL1, XOSC4, FAM46A, FAM98A, FKBP3, FXR2, G3BP2, GLTSCR2, GSPT2, GTF2F1, GTPBP10, HADHB, HDGF, hnRNPE1, HNRPDL, HSPB1, KIAA1324, LARP1, LARP4, LARP4B, LIN28A, LUC7L, MAK16, MATR3, MBNL2, MEPCE, MRPL39, MTDH, NDUFV3, NUFIP2, NUSAP1, PABPC1, PABPC5, PCBP4, PEG10, PPAN, PPIL4, PRPF3, PRPF31, PRRC2B, PTRH1, PUS7, RBM33, RBM38, RBMX2, RPL10A, RPL14, RPL15, RPLPO, RPS20, RPUSD3, RPUSD4, RTN4, SERBP1, SF3A3, SFRS10, SFRS13A, SFRS2IP, SLC7A9, SMN1, SPATS2L, SRSF5, SRSF8, THOC1, TRA2A, TRIM39, TUFM, UBAP2L, UTP23, XPO5, XRN1, YWHAE, or ZRANB2; and
(ii) an antisense oligonucleotide (ASO);
(b) delivering the modulation unit into the cell; and
(c) detecting change in the target RNA translation, wherein the modulation unit modulates translation of the target RNA in the cell.

22. The method of claim 21, wherein the RBP is fused to the ASO.

23. The method of claim 21, wherein the delivering step (b) comprises lipofection.

24. The method of claim 21, wherein the delivering step (b) comprises a virus-based delivery.

25. The method of claim 24, wherein the virus-based delivery comprises adeno-associated virus or lentivirus.

26. The method of claim 21, wherein the target RNA is an endogenous mRNA.

27. The method of claim 21, wherein the target RNA is a non-coding RNA.

28. The method of claim 21, wherein the translation of the target RNA is upregulated.

29. The method of claim 21, wherein the translation of the target RNA is downregulated.

30. The method of claim 21, wherein the modulation unit consists essentially of:
(i) an RNA binding protein (RBP), wherein the RNA binding protein is selected from the group consisting of: AIMP1, BOLL, CLK3, CNOT7, CPEB4, CPSF5, DAZ2, DAZ4, DAZAP1, DDX6, EIF2S2, F3B3S, HSPB1, HNRNPD, IFIT2, LARP1, MEX3C, MTDH, NANOS3, NRNP27, PABPC1, PARN, PLRGI, PRPF3, RBFOX1, SNRPA, SRPR, THOC1, TOB1, TOB2, UBAP2L, or YWHAE; and
(ii) an ASO.


SEQUENCE LISTING (6 sequences)

SEQ ID NO: 1 - RNA, 32 nt, Homo sapiens
  source 1..32: mol_type = genomic RNA
ggcgggtgtggggtccttcccccgcccccccc

SEQ ID NO: 2 - RNA, 47 nt, Homo sapiens
  source 1..47: mol_type = genomic RNA
accggcgatgcggcggcgttattcccatgacccgccgggcagcttcc

SEQ ID NO: 3 - RNA, 21 nt, Homo sapiens
  source 1..21: mol_type = genomic RNA
gggcccccggcggcgggggca

SEQ ID NO: 4 - RNA, 15 nt, Homo sapiens
  source 1..15: mol_type = genomic RNA
tcccccgccccaccc

SEQ ID NO: 5 - RNA, 15 nt, Homo sapiens
  source 1..15: mol_type = genomic RNA
tgccaggtggggagt

SEQ ID NO: 6 - RNA, 14 nt, Homo sapiens
  source 1..14: mol_type = genomic RNA
actctagtctggca